SEQUENCE LISTING (250 sequences)

SEQ ID NO: 1 - protein, 25 aa, synthetic construct
  REGION 1..25: note = PR000325 HFWR1 Chothia
QVQLVQSGAEVKKPGQSLRISCKGA

SEQ ID NO: 2 - protein, 25 aa, synthetic construct
  REGION 1..25: note = PR000400 HFWR1 Chothia
QVQLQQPGAELVRPGASVKLSCKAS

SEQ ID NO: 3 - protein, 25 aa, synthetic construct
  REGION 1..25: note = PR001848 HFWR1 Chothia, PR001861 HFWR1 Chothia, PR002725HFWR1 Chothia, PR002726 HFWR1 Chothia
EVQLLESGGGLVQPGGSLRLSCAAS

SEQ ID NO: 4 - protein, 25 aa, synthetic construct
  REGION 1..25: note = PR003767 HFWR1 Chothia
EVRLQQSGPDLIKPGASVKMSCKAS

SEQ ID NO: 5 - protein, 25 aa, synthetic construct
  REGION 1..25: note = PR003886 HFWR1 Chothia
EVQLVESGGGLVQPGGSLKLSCAAS

SEQ ID NO: 6 - protein, 25 aa, synthetic construct [sequence omitted]

SEQ ID NO: 7 - protein, 25 aa, synthetic construct
  REGION 1..25: note = PR004953 HFWR1 Chothia
EVQLVESGGGVVQPGRSLRLSCAAS

SEQ ID NO: 8 - protein, 26 aa, synthetic construct
  REGION 1..26: note = PR005080 HFWR1 Chothia
QVKLLQSGAALVPKPGDSMKMSCKAS

SEQ ID NO: 9 - protein, 7 aa, synthetic construct
  REGION 1..7: note = PR000325 HCDR1 Chothia
GYSFSTY

SEQ ID NO: 10 - protein, 7 aa, synthetic construct
  REGION 1..7: note = PR000400 HCDR1 Chothia
GYTFTSY

SEQ ID NO: 11 - protein, 7 aa, synthetic construct
  REGION 1..7: note = PR001848 HCDR1 Chothia, PR003886 HCDR1 Chothia
GFTFSTY

SEQ ID NO: 12 - protein, 7 aa, synthetic construct
  REGION 1..7: note = PR001861 HCDR1 Chothia
GFSFSSF

SEQ ID NO: 13 - protein, 7 aa, synthetic construct
  REGION 1..7: note = PR002725 HCDR1 Chothia
GFTFSGY

SEQ ID NO: 14 - protein, 7 aa, synthetic construct
  REGION 1..7: note = PR002726 HCDR1 Chothia
GFTFSSF

SEQ ID NO: 15 - protein, 7 aa, synthetic construct
  REGION 1..7: note = PR003767 HCDR1 Chothia
GYTFTGY

SEQ ID NO: 16 - protein, 7 aa, synthetic construct
  REGION 1..7: note = PR004227 HCDR1 Chothia, PR004533 HCDR1 Chothia, PR004536HCDR1 Chothia, PR004952 HCDR1 Chothia, PR004953 HCDR1Chothia, PR007242 HCDR1 Chothia, PR007243 HCDR1 Chothia,PR007244 HCDR1 Chothia, PR007245
GFTFSSY

SEQ ID NO: 17 - protein, 7 aa, synthetic construct
  REGION 1..7: note = PR004540 HCDR1 Chothia
GFTFSIY

SEQ ID NO: 18 - protein, 7 aa, synthetic construct
  REGION 1..7: note = PR004949 HCDR1 Chothia, PR004950 HCDR1 Chothia
GFTFSAY

SEQ ID NO: 19 - protein, 7 aa, synthetic construct
  REGION 1..7: note = PR005080 HCDR1 Chothia
GYTFTHD

SEQ ID NO: 20 - protein, 19 aa, synthetic construct
  REGION 1..19: note = PR000325 HFWR2 Chothia
WIGWVRQMPGKGLEWMGII

SEQ ID NO: 21 - protein, 19 aa, synthetic construct
  REGION 1..19: note = PR000400 HFWR2 Chothia
WINWVKQRPGQGLEWIGNI

SEQ ID NO: 22 - protein, 19 aa, synthetic construct
  REGION 1..19: note = PR001848 HFWR2 Chothia
AMNWVRQAPGKGLEWVSRI

SEQ ID NO: 23 - protein, 19 aa, synthetic construct
  REGION 1..19: note = PR001861 HFWR2 Chothia
AMSWVRQAPGKGLEWVSAL

SEQ ID NO: 24 - protein, 19 aa, synthetic construct
  REGION 1..19: note = PR002725 HFWR2 Chothia
VMSWVRQAPGKGLEWVSAI

SEQ ID NO: 25 - protein, 19 aa, synthetic construct
  REGION 1..19: note = PR002726 HFWR2 Chothia
VMSWVRQAPGKGLEWVSTI

SEQ ID NO: 26 - protein, 19 aa, synthetic construct
  REGION 1..19: note = PR003767 HFWR2 Chothia
VMHWVKQRPGQGLEWIGFI

SEQ ID NO: 27 - protein, 19 aa, synthetic construct
  REGION 1..19: note = PR003886 HFWR2 Chothia
AMNWVRQASGKGLEWVGRI

SEQ ID NO: 28 - protein, 19 aa, synthetic construct
  REGION 1..19: note = PR004227 HFWR2 Chothia
WMHWVRQAPGKGLEWVSYI

SEQ ID NO: 29 - protein, 19 aa, synthetic construct
  REGION 1..19: note = PR004533 HFWR2 Chothia, PR004950 HFWR2 Chothia, PR007242HFWR2 Chothia, PR007243 HFWR2 Chothia, PR007244 HFWR2Chothia, PR007245 HFWR2 Chothia, PR007246 HFWR2 Chothia,PR007248 HFWR2 Chothia
WMHWVRQVPGKGLVWVSRI

SEQ ID NO: 30 - protein, 19 aa, synthetic construct
  REGION 1..19: note = PR004536 HFWR2 Chothia, PR007247 HFWR2 Chothia
WMYWVRQVPGKGLVWVSHI

SEQ ID NO: 31 - protein, 19 aa, synthetic construct
  REGION 1..19: note = PR004540 HFWR2 Chothia
WMHWVRQVPGKGLVLVSRI

SEQ ID NO: 32 - protein, 19 aa, synthetic construct
  REGION 1..19: note = PR004949 HFWR2 Chothia
WMHWVRQVPGRGLVWVSRI

SEQ ID NO: 33 - protein, 19 aa, synthetic construct
  REGION 1..19: note = PR004952 HFWR2 Chothia
WMHWVRQAPGKGLVWVSRI

SEQ ID NO: 34 - protein, 19 aa, synthetic construct
  REGION 1..19: note = PR004953 HFWR2 Chothia
GMHWVRQAPGKGLEWVAVI

SEQ ID NO: 35 - protein, 19 aa, synthetic construct
  REGION 1..19: note = PR005080 HFWR2 Chothia
IIHWVRQSHGKSLEWIGYI

SEQ ID NO: 36 - protein, 6 aa, synthetic construct
  REGION 1..6: note = PR000325 HCDR2 Chothia
YPDDSD

SEQ ID NO: 37 - protein, 6 aa, synthetic construct
  REGION 1..6: note = PR000400 HCDR2 Chothia
YPSDSY

SEQ ID NO: 38 - protein, 8 aa, synthetic construct
  REGION 1..8: note = PR001848 HCDR2 Chothia, PR003886 HCDR2 Chothia
RSKYNNYA

SEQ ID NO: 39 - protein, 6 aa, synthetic construct
  REGION 1..6: note = PR001861 HCDR2 Chothia, PR002725 HCDR2 Chothia
SGSGGS

SEQ ID NO: 40 - protein, 6 aa, synthetic construct
  REGION 1..6: note = PR002726 HCDR2 Chothia
SGSGRS

SEQ ID NO: 41 - protein, 6 aa, synthetic construct
  REGION 1..6: note = PR003767 HCDR2 Chothia
NPYNDD

SEQ ID NO: 42 - protein, 6 aa, synthetic construct
  REGION 1..6: note = PR004227 HCDR2 Chothia
NNDGSS

SEQ ID NO: 43 - protein, 6 aa, synthetic construct
  REGION 1..6: note = PR004533 HCDR2 Chothia, PR004949 HCDR2 Chothia, PR004950HCDR2 Chothia, PR004952 HCDR2 Chothia
NSDGSR

SEQ ID NO: 44 - protein, 6 aa, synthetic construct
  REGION 1..6: note = PR004536 HCDR2 Chothia
NSDGST

SEQ ID NO: 45 - protein, 6 aa, synthetic construct
  REGION 1..6: note = PR004540 HCDR2 Chothia
SSDGNY

SEQ ID NO: 46 - protein, 6 aa, synthetic construct
  REGION 1..6: note = PR004953 HCDR2 Chothia
GYDGRN

SEQ ID NO: 47 - protein, 6 aa, synthetic construct
  REGION 1..6: note = PR005080 HCDR2 Chothia
NPYNGG

SEQ ID NO: 48 - protein, 6 aa, synthetic construct
  REGION 1..6: note = PR007242 HCDR2 Chothia
NFDSSR

SEQ ID NO: 49 - protein, 6 aa, synthetic construct
  REGION 1..6: note = PR007243 HCDR2 Chothia
SSAGSR

SEQ ID NO: 50 - protein, 6 aa, synthetic construct
  REGION 1..6: note = PR007244 HCDR2 Chothia
DSRGSR

SEQ ID NO: 51 - protein, 6 aa, synthetic construct
  REGION 1..6: note = PR007245 HCDR2 Chothia
SSDASR

SEQ ID NO: 52 - protein, 6 aa, synthetic construct
  REGION 1..6: note = PR007246 HCDR2 Chothia
SSTGSR

SEQ ID NO: 53 - protein, 6 aa, synthetic construct
  REGION 1..6: note = PR007247 HCDR2 Chothia
NPLGST

SEQ ID NO: 54 - protein, 6 aa, synthetic construct
  REGION 1..6: note = PR007248 HCDR2 Chothia
NHDSSR

SEQ ID NO: 55 - protein, 41 aa, synthetic construct
  REGION 1..41: note = PR000325 HFWR3 Chothia
TRYSPSFEGQVTISVDKSITTAYLHWSSLKASDTAIYYCAR

SEQ ID NO: 56 - protein, 41 aa, synthetic construct
  REGION 1..41: note = PR000400 HFWR3 Chothia
TNYNQKFKDKATLTVDKSSSTAYMQLSSPTSEDSAVYYCTR

SEQ ID NO: 57 - protein, 41 aa, synthetic construct
  REGION 1..41: note = PR001848 HFWR3 Chothia
TYYADSVKDRFTISRDDSKSTLYLQMNSLRAEDTAVYYCVR

SEQ ID NO: 58 - protein, 41 aa, synthetic construct
  REGION 1..41: note = PR001861 HFWR3 Chothia
TYYTDSVKGRFTVSRDNSKNTLYLQMNSLRAEDTAIYYCAN

SEQ ID NO: 59 - protein, 41 aa, synthetic construct
  REGION 1..41: note = PR002725 HFWR3 Chothia
KYYADSVKGRFTISRDNSKNTLYLQMNSLRAEDTAVYYCAK

SEQ ID NO: 60 - protein, 41 aa, synthetic construct
  REGION 1..41: note = PR002726 HFWR3 Chothia
TYYADSVKGRFTISRDNSKNTLHLQMNSLRAEDTAVYYCAK

SEQ ID NO: 61 - protein, 41 aa, synthetic construct
  REGION 1..41: note = PR003767 HFWR3 Chothia
IQSNERFRGKATLTSDKSSTTAYMELSSLTSEDSAVYYCAR

SEQ ID NO: 62 - protein, 41 aa, synthetic construct
  REGION 1..41: note = PR003886 HFWR3 Chothia
TYYADSVKDRFTISRDDSKNTAYLQMNSLKTEDTAVYYCTR

SEQ ID NO: 63 - protein, 41 aa, synthetic construct
  REGION 1..41: note = PR004227 HFWR3 Chothia
TRYADSVKGRFTISRDNAKNTLYLQMNSLRAEDTAVYYCTE

SEQ ID NO: 64 - protein, 41 aa, synthetic construct
  REGION 1..41: note = PR004533 HFWR3 Chothia, PR004950 HFWR3 Chothia, PR004952HFWR3 Chothia, PR007242 HFWR3 Chothia, PR007243 HFWR3Chothia, PR007244 HFWR3 Chothia, PR007245 HFWR3 Chothia,PR007246 HFWR3 Chothia, PR007248
TIYADSVKGRFTISRDNAKNTLYLQMNSLRAEDTAVYYCAR

SEQ ID NO: 65 - protein, 41 aa, synthetic construct
  REGION 1..41: note = PR004536 HFWR3 Chothia, PR007247 HFWR3 Chothia
TQYADSVKGRFTISRDNAKNMLYLQMNSLRAEDTAVYYCAR

SEQ ID NO: 66 - protein, 41 aa, synthetic construct
  REGION 1..41: note = PR004540 HFWR3 Chothia
TSYADSVKGRFTISRDNAKNTLYLQMNSLRAEDTAVYLCAR

SEQ ID NO: 67 - protein, 41 aa, synthetic construct
  REGION 1..41: note = PR004949 HFWR3 Chothia
TIYADSVKGRFTISRDNAKNTLYLQMNSLRAEDTAVYLCAR

SEQ ID NO: 68 - protein, 41 aa, synthetic construct
  REGION 1..41: note = PR004953 HFWR3 Chothia
KYYADSVKGRFTISRDNSKNTLFLQMDNLRAEDTALYYCAR

SEQ ID NO: 69 - protein, 41 aa, synthetic construct
  REGION 1..41: note = PR005080 HFWR3 Chothia
TNYNEKFKTKATMTVDKPSSTAYLELTRVSSEASAIYYCAT

SEQ ID NO: 70 - protein, 8 aa, synthetic construct
  REGION 1..8: note = PR000325 HCDR3 Chothia
LVGGAPAY

SEQ ID NO: 71 - protein, 9 aa, synthetic construct
  REGION 1..9: note = PR000400 HCDR3 Chothia
SWRGNSFDY

SEQ ID NO: 72 - protein, 14 aa, synthetic construct
  REGION 1..14: note = PR001848 HCDR3 Chothia, PR003886 HCDR3 Chothia
HGNFGNSYVSWFAY

SEQ ID NO: 73 - protein, 10 aa, synthetic construct
  REGION 1..10: note = PR001861 HCDR3 Chothia
WGGGTGIFEF

SEQ ID NO: 74 - protein, 10 aa, synthetic construct
  REGION 1..10: note = PR002725 HCDR3 Chothia
GDIAVLLFDY

SEQ ID NO: 75 - protein, 11 aa, synthetic construct
  REGION 1..11: note = PR002726 HCDR3 Chothia
DAAAAGTKFDY

SEQ ID NO: 76 - protein, 15 aa, synthetic construct
  REGION 1..15: note = PR003767 HCDR3 Chothia
GAGYNFDGAYRFFDF

SEQ ID NO: 77 - protein, 11 aa, synthetic construct
  REGION 1..11: note = PR004227 HCDR3 Chothia
APPYGNYERDY

SEQ ID NO: 78 - protein, 12 aa, synthetic construct
  REGION 1..12: note = PR004533 HCDR3 Chothia, PR004950 HCDR3 Chothia, PR007242HCDR3 Chothia, PR007243 HCDR3 Chothia, PR007244 HCDR3Chothia, PR007245 HCDR3 Chothia, PR007246 HCDR3 Chothia,PR007248 HCDR3 Chothia
GEDHDILTGYPI

SEQ ID NO: 79 - protein, 14 aa, synthetic construct
  REGION 1..14: note = PR004536 HCDR3 Chothia, PR007247 HCDR3 Chothia
DFPVLGGSHFDFQH

SEQ ID NO: 80 - protein, 12 aa, synthetic construct
  REGION 1..12: note = PR004540 HCDR3 Chothia, PR004949 HCDR3 Chothia
GEDHDMLTGYPV

SEQ ID NO: 81 - protein, 12 aa, synthetic construct
  REGION 1..12: note = PR004952 HCDR3 Chothia
GEDHDILTGYPV

SEQ ID NO: 82 - protein, 14 aa, synthetic construct
  REGION 1..14: note = PR004953 HCDR3 Chothia
HFPSLPGTTDTFDI

SEQ ID NO: 83 - protein, 5 aa, synthetic construct
  REGION 1..5: note = PR005080 HCDR3 Chothia
GSFDY

SEQ ID NO: 84 - protein, 11 aa, synthetic construct
  REGION 1..11: note = PR000325 HFWR4 Chothia, PR001848 HFWR4 Chothia, PR001861HFWR4 Chothia, PR002725 HFWR4 Chothia, PR002726 HFWR4Chothia, PR003886 HFWR4 Chothia, PR004227 HFWR4 Chothia,PR004536 HFWR4 Chothia, PR007247
WGQGTLVTVSS

SEQ ID NO: 85 - protein, 11 aa, synthetic construct
  REGION 1..11: note = PR000400 HFWR4 Chothia, PR003767 HFWR4 Chothia
WGQGTTLTVSS

SEQ ID NO: 86 - protein, 11 aa, synthetic construct
  REGION 1..11: note = PR004533 HFWR4 Chothia, PR004540 HFWR4 Chothia, PR007242HFWR4 Chothia, PR007243 HFWR4 Chothia, PR007244 HFWR4Chothia, PR007245 HFWR4 Chothia, PR007246 HFWR4 Chothia,PR007248 HFWR4 Chothia
RGQGTTVTVSS

SEQ ID NO: 87 - protein, 11 aa, synthetic construct
  REGION 1..11: note = PR004949 HFWR4 Chothia, PR004950 HFWR4 Chothia
RGQGTMVTVSS

SEQ ID NO: 88 - protein, 11 aa, synthetic construct
  REGION 1..11: note = PR004952 HFWR4 Chothia
RGQGATVTVSS

SEQ ID NO: 89 - protein, 11 aa, synthetic construct
  REGION 1..11: note = PR004953 HFWR4 Chothia
RGPGTMVTVSS

SEQ ID NO: 90 - protein, 11 aa, synthetic construct
  REGION 1..11: note = PR005080 HFWR4 Chothia
WGQGVMVTVSS

SEQ ID NO: 91 - protein, 22 aa, synthetic construct
  REGION 1..22: note = PR000325 LFWR1 Chothia
QSALTQPASVSGSPGQSITISC

SEQ ID NO: 92 - protein, 23 aa, synthetic construct
  REGION 1..23: note = PR000400 LFWR1 Chothia
DIVMTQSPSSLTVTAGEKVTMSC

SEQ ID NO: 93 - protein, 22 aa, synthetic construct
  REGION 1..22: note = PR001848 LFWR1 Chothia, PR003886 LFWR1 Chothia
QAVVTQEPSLTVSPGGTVTLTC

SEQ ID NO: 94 - protein, 23 aa, synthetic construct
  REGION 1..23: note = PR001861 LFWR1 Chothia
EIVMTQSPATLSVSPGERATLSC

SEQ ID NO: 95 - protein, 23 aa, synthetic construct
  REGION 1..23: note = PR002725 LFWR1 Chothia
EMVMTQSPATLSVSPGERATLSC

SEQ ID NO: 96 - protein, 23 aa, synthetic construct
  REGION 1..23: note = PR002726 LFWR1 Chothia
EIVLTQSPATLSLSPGERATLSC

SEQ ID NO: 97 - protein, 23 aa, synthetic construct
  REGION 1..23: note = PR003767 LFWR1 Chothia
DVVMTQSPLSLPVSLGDQASISC

SEQ ID NO: 98 - protein, 23 aa, synthetic construct
  REGION 1..23: note = PR005080 LFWR1 Chothia
DVVLTQTPGSLSLAIGQSASISC

SEQ ID NO: 99 - protein, 14 aa, synthetic construct
  REGION 1..14: note = PR000325 LCDR1 Chothia
SGSSSDIGRYDYVS

SEQ ID NO: 100 - protein, 17 aa, synthetic construct
  REGION 1..17: note = PR000400 LCDR1 Chothia
KSSQSLLNSGNQKNYLT

SEQ ID NO: 101 - protein, 14 aa, synthetic construct
  REGION 1..14: note = PR001848 LCDR1 Chothia, PR003886 LCDR1 Chothia
RSSTGAVTTSNYAN

SEQ ID NO: 102 - protein, 11 aa, synthetic construct
  REGION 1..11: note = PR001861 LCDR1 Chothia
RASQSVRSYLA

SEQ ID NO: 103 - protein, 11 aa, synthetic construct
  REGION 1..11: note = PR002725 LCDR1 Chothia
RASQSVSRNLA

SEQ ID NO: 104 - protein, 11 aa, synthetic construct
  REGION 1..11: note = PR002726 LCDR1 Chothia
RASQSVSSYLA

SEQ ID NO: 105 - protein, 16 aa, synthetic construct
  REGION 1..16: note = PR003767 LCDR1 Chothia
RSSQRLVHSNGNTYLH

SEQ ID NO: 106 - protein, 16 aa, synthetic construct
  REGION 1..16: note = PR005080 LCDR1 Chothia
KSSQSLLGTSGKTFLN

SEQ ID NO: 107 - protein, 15 aa, synthetic construct
  REGION 1..15: note = PR000325 LFWR2 Chothia
WYQHYPDKAPKLIIY

SEQ ID NO: 108 - protein, 15 aa, synthetic construct
  REGION 1..15: note = PR000400 LFWR2 Chothia
WYQQKPGQPPKLLIY

SEQ ID NO: 109 - protein, 15 aa, synthetic construct
  REGION 1..15: note = PR001848 LFWR2 Chothia, PR003886 LFWR2 Chothia
WVQQKPGQAPRGLIG

SEQ ID NO: 110 - protein, 15 aa, synthetic construct
  REGION 1..15: note = PR001861 LFWR2 Chothia, PR002726 LFWR2 Chothia
WYQQKPGQAPRLLIY

SEQ ID NO: 111 - protein, 15 aa, synthetic construct
  REGION 1..15: note = PR002725 LFWR2 Chothia
WFQQKPGQAPRLLIY

SEQ ID NO: 112 - protein, 15 aa, synthetic construct
  REGION 1..15: note = PR003767 LFWR2 Chothia
WYLQKPGQSPKLLIY

SEQ ID NO: 113 - protein, 15 aa, synthetic construct
  REGION 1..15: note = PR005080 LFWR2 Chothia
WILQRPGQSPERLIY

SEQ ID NO: 114 - protein, 7 aa, synthetic construct
  REGION 1..7: note = PR000325 LCDR2 Chothia
EVKHRPS

SEQ ID NO: 115 - protein, 7 aa, synthetic construct
  REGION 1..7: note = PR000400 LCDR2 Chothia
WASTRES

SEQ ID NO: 116 - protein, 7 aa, synthetic construct
  REGION 1..7: note = PR001848 LCDR2 Chothia, PR003886 LCDR2 Chothia
GTNKRAP

SEQ ID NO: 117 - protein, 7 aa, synthetic construct
  REGION 1..7: note = PR001861 LCDR2 Chothia, PR002725 LCDR2 Chothia
GASTRAT

SEQ ID NO: 118 - protein, 7 aa, synthetic construct
  REGION 1..7: note = PR002726 LCDR2 Chothia
DASNRAT

SEQ ID NO: 119 - protein, 7 aa, synthetic construct
  REGION 1..7: note = PR003767 LCDR2 Chothia
RVSNRFP

SEQ ID NO: 120 - protein, 7 aa, synthetic construct
  REGION 1..7: note = PR005080 LCDR2 Chothia
QVSKLYS

SEQ ID NO: 121 - protein, 32 aa, synthetic construct
  REGION 1..32: note = PR000325 LFWR3 Chothia
GISHRFSASKSGNTASLTISELQPGDEADYYC

SEQ ID NO: 122 - protein, 32 aa, synthetic construct
  REGION 1..32: note = PR000400 LFWR3 Chothia
GVPDRFTGSGSGTDFTLTISSVQAEDLAVYYC

SEQ ID NO: 123 - protein, 32 aa, synthetic construct
  REGION 1..32: note = PR001848 LFWR3 Chothia, PR003886 LFWR3 Chothia
WTPARFSGSLLGDKAALTLLGAQPEDEAEYFC

SEQ ID NO: 124 - protein, 32 aa, synthetic construct
  REGION 1..32: note = PR001861 LFWR3 Chothia
GIPARFSGSGSGTEFTLTISSLQSEDFAVYHC

SEQ ID NO: 125 - protein, 32 aa, synthetic construct
  REGION 1..32: note = PR002725 LFWR3 Chothia
GIPARFSGSGSGTEFTLTISSLQSEDFAVYYC

SEQ ID NO: 126 - protein, 32 aa, synthetic construct
  REGION 1..32: note = PR002726 LFWR3 Chothia
GIPARFSGSGSGTDFTLTISSLEPEDFAVYYC

SEQ ID NO: 127 - protein, 32 aa, synthetic construct
  REGION 1..32: note = PR003767 LFWR3 Chothia
GVPDRFSGSGSGTDFTLKISRVEAEDLGIYFC

SEQ ID NO: 128 - protein, 32 aa, synthetic construct
  REGION 1..32: note = PR005080 LFWR3 Chothia
EVPDRFSGSGSETEFTLKISRVEAEDLGVYYC

SEQ ID NO: 129 - protein, 10 aa, synthetic construct
  REGION 1..10: note = PR000325 LCDR3 Chothia
ASYTESKTYI

SEQ ID NO: 130 - protein, 9 aa, synthetic construct
  REGION 1..9: note = PR000400 LCDR3 Chothia
QNDYSYPFT

SEQ ID NO: 131 - protein, 9 aa, synthetic construct
  REGION 1..9: note = PR001848 LCDR3 Chothia, PR003886 LCDR3 Chothia
ALWYSNLWV

SEQ ID NO: 132 - protein, 9 aa, synthetic construct
  REGION 1..9: note = PR001861 LCDR3 Chothia, PR002725 LCDR3 Chothia
QQYNNWPLT

SEQ ID NO: 133 - protein, 9 aa, synthetic construct
  REGION 1..9: note = PR002726 LCDR3 Chothia
QQRSNWPLT

SEQ ID NO: 134 - protein, 9 aa, synthetic construct
  REGION 1..9: note = PR003767 LCDR3 Chothia
SQSTHVPYT

SEQ ID NO: 135 - protein, 9 aa, synthetic construct
  REGION 1..9: note = PR005080 LCDR3 Chothia
WQGIHFPHT

SEQ ID NO: 136 - protein, 10 aa, synthetic construct
  REGION 1..10: note = PR000325 LFWR4 Chothia
FGGGTKVTVL

SEQ ID NO: 137 - protein, 10 aa, synthetic construct
  REGION 1..10: note = PR000400 LFWR4 Chothia
FGSGTKLEIK

SEQ ID NO: 138 - protein, 10 aa, synthetic construct
  REGION 1..10: note = PR001848 LFWR4 Chothia, PR003886 LFWR4 Chothia
FGGGTKLTVL

SEQ ID NO: 139 - protein, 10 aa, synthetic construct
  REGION 1..10: note = PR001861 LFWR4 Chothia, PR002725 LFWR4 Chothia, PR002726LFWR4 Chothia
FGGGTKVEIK

SEQ ID NO: 140 - protein, 10 aa, synthetic construct
  REGION 1..10: note = PR003767 LFWR4 Chothia
FGGGTKLEIK

SEQ ID NO: 141 - protein, 10 aa, synthetic construct
  REGION 1..10: note = PR005080 LFWR4 Chothia
FGAGTKLELK

SEQ ID NO: 142 - protein, 117 aa, synthetic construct
  REGION 1..117: note = PR000325 VH
QVQLVQSGAEVKKPGQSLRISCKGAGYSFSTYWIGWVRQMPGKGLEWMGIIYPDDSDTRYSPSFEGQVTISVDKSITTAYLHWSSLKASDTAIYYCARLVGGAPAYWGQGTLVTVSS

SEQ ID NO: 143 - protein, 118 aa, synthetic construct
  REGION 1..118: note = PR000400 VH
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 144 - protein, 125 aa, synthetic construct
  REGION 1..125: note = PR001848 VH
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSS

SEQ ID NO: 145 - protein, 119 aa, synthetic construct
  REGION 1..119: note = PR001861 VH
EVQLLESGGGLVQPGGSLRLSCAASGFSFSSFAMSWVRQAPGKGLEWVSALSGSGGSTYYTDSVKGRFTVSRDNSKNTLYLQMNSLRAEDTAIYYCANWGGGTGIFEFWGQGTLVTVSS

SEQ ID NO: 146 - protein, 119 aa, synthetic construct
  REGION 1..119: note = PR002725 VH
EVQLLESGGGLVQPGGSLRLSCAASGFTFSGYVMSWVRQAPGKGLEWVSAISGSGGSKYYADSVKGRFTISRDNSKNTLYLQMNSLRAEDTAVYYCAKGDIAVLLFDYWGQGTLVTVSS

SEQ ID NO: 147 - protein, 120 aa, synthetic construct
  REGION 1..120: note = PR002726 VH
EVQLLESGGGLVQPGGSLRLSCAASGFTFSSFVMSWVRQAPGKGLEWVSTISGSGRSTYYADSVKGRFTISRDNSKNTLHLQMNSLRAEDTAVYYCAKDAAAAGTKFDYWGQGTLVTVSS

SEQ ID NO: 148 - protein, 124 aa, synthetic construct
  REGION 1..124: note = PR003767 VH
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSS

SEQ ID NO: 149 - protein, 125 aa, synthetic construct
  REGION 1..125: note = PR003886 VH
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 150 - protein, 120 aa, synthetic construct
  REGION 1..120: note = PR004227 VH
EVQLVESGGGLVQPGGSLRLSCAASGFTFSSYWMHWVRQAPGKGLEWVSYINNDGSSTRYADSVKGRFTISRDNAKNTLYLQMNSLRAEDTAVYYCTEAPPYGNYERDYWGQGTLVTVSS

SEQ ID NO: 151 - protein, 121 aa, synthetic construct
  REGION 1..121: note = PR004533 VH
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSS

SEQ ID NO: 152 - protein, 123 aa, synthetic construct
  REGION 1..123: note = PR004536 VH
EVQLVESGGGLVQPGGSLRLSCAASGFTFSSYWMYWVRQVPGKGLVWVSHINSDGSTTQYADSVKGRFTISRDNAKNMLYLQMNSLRAEDTAVYYCARDFPVLGGSHFDFQHWGQGTLVTVSS

SEQ ID NO: 153 - protein, 121 aa, synthetic construct
  REGION 1..121: note = PR004540 VH
EVQLVESGGGLVQPGGSLRLSCAASGFTFSIYWMHWVRQVPGKGLVLVSRISSDGNYTSYADSVKGRFTISRDNAKNTLYLQMNSLRAEDTAVYLCARGEDHDMLTGYPVRGQGTTVTVSS

SEQ ID NO: 154 - protein, 121 aa, synthetic construct
  REGION 1..121: note = PR004949 VH
EVQLVESGGGLVQPGGSLRLSCAASGFTFSAYWMHWVRQVPGRGLVWVSRINSDGSRTIYADSVKGRFTISRDNAKNTLYLQMNSLRAEDTAVYLCARGEDHDMLTGYPVRGQGTMVTVSS

SEQ ID NO: 155 - protein, 121 aa, synthetic construct
  REGION 1..121: note = PR004950 VH
EVQLVESGGGLVQPGGSLRLSCAASGFTFSAYWMHWVRQVPGKGLVWVSRINSDGSRTIYADSVKGRFTISRDNAKNTLYLQMNSLRAEDTAVYYCARGEDHDILTGYPIRGQGTMVTVSS

SEQ ID NO: 156 - protein, 121 aa, synthetic construct
  REGION 1..121: note = PR004952 VH
EVQLVESGGGLVQPGGSLRLSCAASGFTFSSYWMHWVRQAPGKGLVWVSRINSDGSRTIYADSVKGRFTISRDNAKNTLYLQMNSLRAEDTAVYYCARGEDHDILTGYPVRGQGATVTVSS

SEQ ID NO: 157 - protein, 123 aa, synthetic construct
  REGION 1..123: note = PR004953 VH
EVQLVESGGGVVQPGRSLRLSCAASGFTFSSYGMHWVRQAPGKGLEWVAVIGYDGRNKYYADSVKGRFTISRDNSKNTLFLQMDNLRAEDTALYYCARHFPSLPGTTDTFDIRGPGTMVTVSS

SEQ ID NO: 158 - protein, 115 aa, synthetic construct
  REGION 1..115: note = PR005080 VH
QVKLLQSGAALVPKPGDSMKMSCKASGYTFTHDIIHWVRQSHGKSLEWIGYINPYNGGTNYNEKFKTKATMTVDKPSSTAYLELTRVSSEASAIYYCATGSFDYWGQGVMVTVSS

SEQ ID NO: 159 - protein, 121 aa, synthetic construct
  REGION 1..121: note = PR007242 VH
EVQLVESGGGLVQPGGSLRLSCAASGFTFSSYWMHWVRQVPGKGLVWVSRINFDSSRTIYADSVKGRFTISRDNAKNTLYLQMNSLRAEDTAVYYCARGEDHDILTGYPIRGQGTTVTVSS

SEQ ID NO: 160 - protein, 121 aa, synthetic construct
  REGION 1..121: note = PR007243 VH
EVQLVESGGGLVQPGGSLRLSCAASGFTFSSYWMHWVRQVPGKGLVWVSRISSAGSRTIYADSVKGRFTISRDNAKNTLYLQMNSLRAEDTAVYYCARGEDHDILTGYPIRGQGTTVTVSS

SEQ ID NO: 161 - protein, 121 aa, synthetic construct
  REGION 1..121: note = PR007244 VH
EVQLVESGGGLVQPGGSLRLSCAASGFTFSSYWMHWVRQVPGKGLVWVSRIDSRGSRTIYADSVKGRFTISRDNAKNTLYLQMNSLRAEDTAVYYCARGEDHDILTGYPIRGQGTTVTVSS

SEQ ID NO: 162 - protein, 121 aa, synthetic construct
  REGION 1..121: note = PR007245 VH
EVQLVESGGGLVQPGGSLRLSCAASGFTFSSYWMHWVRQVPGKGLVWVSRISSDASRTIYADSVKGRFTISRDNAKNTLYLQMNSLRAEDTAVYYCARGEDHDILTGYPIRGQGTTVTVSS

SEQ ID NO: 163 - protein, 121 aa, synthetic construct
  REGION 1..121: note = PR007246 VH
EVQLVESGGGLVQPGGSLRLSCAASGFTFSSYWMHWVRQVPGKGLVWVSRISSTGSRTIYADSVKGRFTISRDNAKNTLYLQMNSLRAEDTAVYYCARGEDHDILTGYPIRGQGTTVTVSS

SEQ ID NO: 164 - protein, 123 aa, synthetic construct
  REGION 1..123: note = PR007247 VH
EVQLVESGGGLVQPGGSLRLSCAASGFTFSSYWMYWVRQVPGKGLVWVSHINPLGSTTQYADSVKGRFTISRDNAKNMLYLQMNSLRAEDTAVYYCARDFPVLGGSHFDFQHWGQGTLVTVSS

SEQ ID NO: 165 - protein, 121 aa, synthetic construct
  REGION 1..121: note = PR007248 VH
EVQLVESGGGLVQPGGSLRLSCAASGFTFSSYWMHWVRQVPGKGLVWVSRINHDSSRTIYADSVKGRFTISRDNAKNTLYLQMNSLRAEDTAVYYCARGEDHDILTGYPIRGQGTTVTVSS

SEQ ID NO: 166 - protein, 110 aa, synthetic construct
  REGION 1..110: note = PR000325 VL
QSALTQPASVSGSPGQSITISCSGSSSDIGRYDYVSWYQHYPDKAPKLIIYEVKHRPSGISHRFSASKSGNTASLTISELQPGDEADYYCASYTESKTYIFGGGTKVTVL

SEQ ID NO: 167 - protein, 113 aa, synthetic construct
  REGION 1..113: note = PR000400 VL
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 168 - protein, 109 aa, synthetic construct
  REGION 1..109: note = PR001848 VL, PR003886 VL
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVL

SEQ ID NO: 169 - protein, 107 aa, synthetic construct
  REGION 1..107: note = PR001861 VL
EIVMTQSPATLSVSPGERATLSCRASQSVRSYLAWYQQKPGQAPRLLIYGASTRATGIPARFSGSGSGTEFTLTISSLQSEDFAVYHCQQYNNWPLTFGGGTKVEIK

SEQ ID NO: 170 - protein, 107 aa, synthetic construct
  REGION 1..107: note = PR002725 VL
EMVMTQSPATLSVSPGERATLSCRASQSVSRNLAWFQQKPGQAPRLLIYGASTRATGIPARFSGSGSGTEFTLTISSLQSEDFAVYYCQQYNNWPLTFGGGTKVEIK

SEQ ID NO: 171 - protein, 107 aa, synthetic construct
  REGION 1..107: note = PR002726 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 172 - protein, 112 aa, synthetic construct
  REGION 1..112: note = PR003767 VL
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIK

SEQ ID NO: 173 - protein, 112 aa, synthetic construct
  REGION 1..112: note = PR005080 VL
DVVLTQTPGSLSLAIGQSASISCKSSQSLLGTSGKTFLNWILQRPGQSPERLIYQVSKLYSEVPDRFSGSGSETEFTLKISRVEAEDLGVYYCWQGIHFPHTFGAGTKLELK

SEQ ID NO: 174 - protein, 447 aa, synthetic construct
  REGION 1..447: note = PR000325 HC
QVQLVQSGAEVKKPGQSLRISCKGAGYSFSTYWIGWVRQMPGKGLEWMGIIYPDDSDTRYSPSFEGQVTISVDKSITTAYLHWSSLKASDTAIYYCARLVGGAPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 448 aa, synthetic construct
  REGION 1..448: note = PR000400 HC
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 455 aa, synthetic construct
  REGION 1..455: note = PR001848 HC
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 449 aa, synthetic construct
  REGION 1..449: note = PR001861 HC
EVQLLESGGGLVQPGGSLRLSCAASGFSFSSFAMSWVRQAPGKGLEWVSALSGSGGSTYYTDSVKGRFTVSRDNSKNTLYLQMNSLRAEDTAIYYCANWGGGTGIFEFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 449 aa, synthetic construct
  REGION 1..449: note = PR002725 HC
EVQLLESGGGLVQPGGSLRLSCAASGFTFSGYVMSWVRQAPGKGLEWVSAISGSGGSKYYADSVKGRFTISRDNSKNTLYLQMNSLRAEDTAVYYCAKGDIAVLLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 450 aa, synthetic construct
  REGION 1..450: note = PR002726 HC
EVQLLESGGGLVQPGGSLRLSCAASGFTFSSFVMSWVRQAPGKGLEWVSTISGSGRSTYYADSVKGRFTISRDNSKNTLHLQMNSLRAEDTAVYYCAKDAAAAGTKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 454 aa, synthetic construct
  REGION 1..454: note = PR003767 HC
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 455 aa, synthetic construct
  REGION 1..455: note = PR003886 HC
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 357 aa, synthetic construct
  REGION 1..357: note = PR004227 HC
EVQLVESGGGLVQPGGSLRLSCAASGFTFSSYWMHWVRQAPGKGLEWVSYINNDGSSTRYADSVKGRFTISRDNAKNTLYLQMNSLRAEDTAVYYCTEAPPYGNYERDYWGQGTLVTVSSGQAGQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 353 aa, synthetic construct
  REGION 1..353: note = PR004533 HC
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 355 aa, synthetic construct
  REGION 1..355: note = PR004536 HC
EVQLVESGGGLVQPGGSLRLSCAASGFTFSSYWMYWVRQVPGKGLVWVSHINSDGSTTQYADSVKGRFTISRDNAKNMLYLQMNSLRAEDTAVYYCARDFPVLGGSHFDFQHWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 353 aa, synthetic construct
  REGION 1..353: note = PR004540 HC
EVQLVESGGGLVQPGGSLRLSCAASGFTFSIYWMHWVRQVPGKGLVLVSRISSDGNYTSYADSVKGRFTISRDNAKNTLYLQMNSLRAEDTAVYLCARGEDHDMLTGYPVRGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 353 aa, synthetic construct
  REGION 1..353: note = PR004949 HC
EVQLVESGGGLVQPGGSLRLSCAASGFTFSAYWMHWVRQVPGRGLVWVSRINSDGSRTIYADSVKGRFTISRDNAKNTLYLQMNSLRAEDTAVYLCARGEDHDMLTGYPVRGQGTMVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 353 aa, synthetic construct
  REGION 1..353: note = PR004950 HC
EVQLVESGGGLVQPGGSLRLSCAASGFTFSAYWMHWVRQVPGKGLVWVSRINSDGSRTIYADSVKGRFTISRDNAKNTLYLQMNSLRAEDTAVYYCARGEDHDILTGYPIRGQGTMVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 353 aa, synthetic construct
  REGION 1..353: note = PR004952 HC
EVQLVESGGGLVQPGGSLRLSCAASGFTFSSYWMHWVRQAPGKGLVWVSRINSDGSRTIYADSVKGRFTISRDNAKNTLYLQMNSLRAEDTAVYYCARGEDHDILTGYPVRGQGATVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 355 aa, synthetic construct
  REGION 1..355: note = PR004953 HC
EVQLVESGGGVVQPGRSLRLSCAASGFTFSSYGMHWVRQAPGKGLEWVAVIGYDGRNKYYADSVKGRFTISRDNSKNTLFLQMDNLRAEDTALYYCARHFPSLPGTTDTFDIRGPGTMVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 445 aa, synthetic construct
  REGION 1..445: note = PR005080 HC
QVKLLQSGAALVPKPGDSMKMSCKASGYTFTHDIIHWVRQSHGKSLEWIGYINPYNGGTNYNEKFKTKATMTVDKPSSTAYLELTRVSSEASAIYYCATGSFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 353 aa, synthetic construct
  REGION 1..353: note = PR007242 HC
EVQLVESGGGLVQPGGSLRLSCAASGFTFSSYWMHWVRQVPGKGLVWVSRINFDSSRTIYADSVKGRFTISRDNAKNTLYLQMNSLRAEDTAVYYCARGEDHDILTGYPIRGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 353 aa, synthetic construct
  REGION 1..353: note = PR007243 HC
EVQLVESGGGLVQPGGSLRLSCAASGFTFSSYWMHWVRQVPGKGLVWVSRISSAGSRTIYADSVKGRFTISRDNAKNTLYLQMNSLRAEDTAVYYCARGEDHDILTGYPIRGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 353 aa, synthetic construct
  REGION 1..353: note = PR007244 HC
EVQLVESGGGLVQPGGSLRLSCAASGFTFSSYWMHWVRQVPGKGLVWVSRIDSRGSRTIYADSVKGRFTISRDNAKNTLYLQMNSLRAEDTAVYYCARGEDHDILTGYPIRGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 353 aa, synthetic construct
  REGION 1..353: note = PR007245 HC
EVQLVESGGGLVQPGGSLRLSCAASGFTFSSYWMHWVRQVPGKGLVWVSRISSDASRTIYADSVKGRFTISRDNAKNTLYLQMNSLRAEDTAVYYCARGEDHDILTGYPIRGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 353 aa, synthetic construct
  REGION 1..353: note = PR007246 HC
EVQLVESGGGLVQPGGSLRLSCAASGFTFSSYWMHWVRQVPGKGLVWVSRISSTGSRTIYADSVKGRFTISRDNAKNTLYLQMNSLRAEDTAVYYCARGEDHDILTGYPIRGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 355 aa, synthetic construct
  REGION 1..355: note = PR007247 HC
EVQLVESGGGLVQPGGSLRLSCAASGFTFSSYWMYWVRQVPGKGLVWVSHINPLGSTTQYADSVKGRFTISRDNAKNMLYLQMNSLRAEDTAVYYCARDFPVLGGSHFDFQHWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 353 aa, synthetic construct
  REGION 1..353: note = PR007248 HC
EVQLVESGGGLVQPGGSLRLSCAASGFTFSSYWMHWVRQVPGKGLVWVSRINHDSSRTIYADSVKGRFTISRDNAKNTLYLQMNSLRAEDTAVYYCARGEDHDILTGYPIRGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 216 aa, synthetic construct
  REGION 1..216: note = PR000325 LC
QSALTQPASVSGSPGQSITISCSGSSSDIGRYDYVSWYQHYPDKAPKLIIYEVKHRPSGISHRFSASKSGNTASLTISELQPGDEADYYCASYTESKTYIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 199 - protein, 220 aa, synthetic construct
  REGION 1..220: note = PR000400 LC
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 215 aa, synthetic construct
  REGION 1..215: note = PR001848 LC, PR003886 LC, PR004312 Chain, PR004313Chain, PR004603 Chain, PR005072 Chain, PR005076 Chain,PR005354 Chain, PR005397 Chain, PR005398 Chain, PR005399Chain, PR005401 Chain, PR005411 Chain
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 201 - protein, 214 aa, synthetic construct
  REGION 1..214: note = PR001861 LC
EIVMTQSPATLSVSPGERATLSCRASQSVRSYLAWYQQKPGQAPRLLIYGASTRATGIPARFSGSGSGTEFTLTISSLQSEDFAVYHCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 214 aa, synthetic construct
  REGION 1..214: note = PR002725 LC
EMVMTQSPATLSVSPGERATLSCRASQSVSRNLAWFQQKPGQAPRLLIYGASTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 214 aa, synthetic construct
  REGION 1..214: note = PR002726 LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 219 aa, synthetic construct
  REGION 1..219: note = PR003767 LC
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 219 aa, synthetic construct
  REGION 1..219: note = PR005080 LC
DVVLTQTPGSLSLAIGQSASISCKSSQSLLGTSGKTFLNWILQRPGQSPERLIYQVSKLYSEVPDRFSGSGSETEFTLKISRVEAEDLGVYYCWQGIHFPHTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 214 aa, synthetic construct
  REGION 1..214: note = PR002199 Chain
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 453 aa, synthetic construct
  REGION 1..453: note = PR002199 Chain
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 477 aa, synthetic construct
  REGION 1..477: note = PR002199 Chain
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 209 - protein, 455 aa, synthetic construct
  REGION 1..455: note = PR004312 Chain, PR004313 Chain, PR005354 Chain, PR006023Chain
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 481 aa, synthetic construct
  REGION 1..481: note = PR004312 Chain
QVQLVQSGAEVKKPGQSLRISCKGAGYSFSTYWIGWVRQMPGKGLEWMGIIYPDDSDTRYSPSFEGQVTISVDKSITTAYLHWSSLKASDTAIYYCARLVGGAPAYWGQGTLVTVSSGGGGSGGGGSGGGGSGGGGSQSALTQPASVSGSPGQSITISCSGSSSDIGRYDYVSWYQHYPDKAPKLIIYEVKHRPSGISHRFSASKSGNTASLTISELQPGDEADYYCASYTESKTYIFGGGTKVTVLASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 211 - protein, 480 aa, synthetic construct
  REGION 1..480: note = PR004313 Chain
EVQLLESGGGLVQPGGSLRLSCAASGFSFSSFAMSWVRQAPGKGLEWVSALSGSGGSTYYTDSVKGRFTVSRDNSKNTLYLQMNSLRAEDTAIYYCANWGGGTGIFEFWGQGTLVTVSSGGGGSGGGGSGGGGSGGGGSEIVMTQSPATLSVSPGERATLSCRASQSVRSYLAWYQQKPGQAPRLLIYGASTRATGIPARFSGSGSGTEFTLTISSLQSEDFAVYHCQQYNNWPLTFGGGTKVEIKASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 212 - protein, 988 aa, synthetic construct
  REGION 1..988: note = PR004549 Chain
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 455 aa, synthetic construct
  REGION 1..455: note = PR004603 Chain, PR005072 Chain, PR005076 Chain, PR005397Chain, PR005398 Chain, PR005399 Chain, PR005401 Chain,PR005411 Chain, PR005422 Chain, PR005525 Chain
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 489 aa, synthetic construct
  REGION 1..489: note = PR004603 Chain
EVQLVESGGGLVQPGGSLRLSCAASGFTFSSYWMHWVRQAPGKGLEWVSYINNDGSSTRYADSVKGRFTISRDNAKNTLYLQMNSLRAEDTAVYYCTEAPPYGNYERDYWGQGTLVTVSSGGGGSGGGGSGGGGSEVQLVESGGGLVQPGGSLRLSCAASGFTFSSYWMHWVRQAPGKGLEWVSYINNDGSSTRYADSVKGRFTISRDNAKNTLYLQMNSLRAEDTAVYYCTEAPPYGNYERDYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 215 - protein, 214 aa, synthetic construct
  REGION 1..214: note = PR004931 Chain
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKESNTKVDKKVEPKSC

SEQ ID NO: 216 - protein, 215 aa, synthetic construct
  REGION 1..215: note = PR004931 Chain
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNEYPREAKVQWKVDNALQSGNSQESVTEQDSKDSTYSLSSTLTLSKADYEKHKVYACEVTHQGLSSPVTKSFNRGEC

SEQ ID NO: 217 - protein, 451 aa, synthetic construct
  REGION 1..451: note = PR004931 Chain
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 218 - protein, 694 aa, synthetic construct
  REGION 1..694: note = PR004931 Chain
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 491 aa, synthetic construct
  REGION 1..491: note = PR005072 Chain, PR005354 Chain, PR005520 Chain, PR005521Chain, PR005522 Chain
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 220 - protein, 495 aa, synthetic construct
  REGION 1..495: note = PR005076 Chain
EVQLVESGGGLVQPGGSLRLSCAASGFTFSSYWMYWVRQVPGKGLVWVSHINSDGSTTQYADSVKGRFTISRDNAKNMLYLQMNSLRAEDTAVYYCARDFPVLGGSHFDFQHWGQGTLVTVSSGGGGSGGGGSGGGGSEVQLVESGGGLVQPGGSLRLSCAASGFTFSSYWMYWVRQVPGKGLVWVSHINSDGSTTQYADSVKGRFTISRDNAKNMLYLQMNSLRAEDTAVYYCARDFPVLGGSHFDFQHWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 221 - protein, 491 aa, synthetic construct
  REGION 1..491: note = PR005397 Chain, PR006023 Chain
EVQLVESGGGLVQPGGSLRLSCAASGFTFSSYWMHWVRQVPGKGLVWVSRISSTGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRISSTGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 222 - protein, 491 aa, synthetic construct
  REGION 1..491: note = PR005398 Chain
EVQLVESGGGLVQPGGSLRLSCAASGFTFSSYWMHWVRQVPGKGLVWVSRINFDSSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRINFDSSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 223 - protein, 491 aa, synthetic construct
  REGION 1..491: note = PR005399 Chain
EVQLVESGGGLVQPGGSLRLSCAASGFTFSSYWMHWVRQVPGKGLVWVSRISSAGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRISSAGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 224 - protein, 491 aa, synthetic construct
  REGION 1..491: note = PR005401 Chain
EVQLVESGGGLVQPGGSLRLSCAASGFTFSSYWMHWVRQVPGKGLVWVSRIDSRGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRIDSRGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 225 - protein, 491 aa, synthetic construct
  REGION 1..491: note = PR005411 Chain
EVQLVESGGGLVQPGGSLRLSCAASGFTFSSYWMHWVRQVPGKGLVWVSRISSDASRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRISSDASRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 226 - protein, 495 aa, synthetic construct
  REGION 1..495: note = PR005422 Chain
EVQLVESGGGLVQPGGSLRLSCAASGFTFSSYWMYWVRQVPGKGLVWVSHINPLGSTTQYADSVKGRFTISRDNAKNMLYLQMNSLRAEDTAVYYCARDFPVLGGSHFDFQHWGQGTLVTVSSGGGGSGGGGSGGGGSEVQLVESGGGLVQPGGSLRLSCAASGFTFSSYWMYWVRQVPGKGLVWVSHINPLGSTTQYADSVKGRFTISRDNAKNMLYLQMNSLRAEDTAVYYCARDFPVLGGSHFDFQHWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 227 - protein, 355 aa, synthetic construct
  REGION 1..355: note = PR005518 Chain, PR005519 Chain
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 228 - protein, 599 aa, synthetic construct
  REGION 1..599: note = PR005518 Chain
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSRTEVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 229 - protein, 228 aa, synthetic construct
  REGION 1..228: note = PR005519 Chain, PR005521 Chain
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 230 - protein, 586 aa, synthetic construct
  REGION 1..586: note = PR005519 Chain
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSRTQAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 231 - protein, 735 aa, synthetic construct
  REGION 1..735: note = PR005520 Chain
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSRTEVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 232 - protein, 722 aa, synthetic construct
  REGION 1..722: note = PR005521 Chain
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSRTQAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 233 - protein, 591 aa, synthetic construct
  REGION 1..591: note = PR005522 Chain
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSGGGGSGGGGSGGGGSEVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 627 aa, synthetic construct
  REGION 1..627: note = PR005525 Chain
EVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRINSDGSRTIYADSVKGRFTISRDNAKNTLYLQMNSLRAEDTAVYYCARGEDHDILTGYPIRGQGTTVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 235 - protein, 455 aa, synthetic construct
  REGION 1..455: note = PR006292 Chain, PR006384 Chain, PR007079 Chain, PR007080Chain, PR007081 Chain, PR007082 Chain, PR007083 Chain
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 491 aa, synthetic construct
  REGION 1..491: note = PR006292 Chain, PR006293 Chain
EVQLVESGGGLVQPGGSLRLSCAASGFTFSSYWMHWVRQVPGKGLVWVSRISSDASRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRISSDASRTIYADSVKGRFTISRDNAKNTLYLQMNSLRAEDTAVYYCARGEDHDILTGYPIRGQGTTVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 237 - protein, 455 aa, synthetic construct
  REGION 1..455: note = PR006293 Chain
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 491 aa, synthetic construct
  REGION 1..491: note = PR006384 Chain
EVQLVESGGGLVQPGGSLRLSCAASGFTFSSYWMHWVRQVPGKGLVWVSRISSTGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRISSTGSRTIYADSVKGRFTISRDNAKNTLYLQMNSLRAEDTAVYYCARGEDHDILTGYPIRGQGTTVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 239 - protein, 491 aa, synthetic construct
  REGION 1..491: note = PR007079 Chain
EVQLVESGGGLVQPGGSLRLSCAASGFTFSSYWMHWVRQVPGKGLVWVSRISSAGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRISSAGSRTIYADSVKGRFTISRDNAKNTLYLQMNSLRAEDTAVYYCARGEDHDILTGYPIRGQGTTVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 240 - protein, 491 aa, synthetic construct
  REGION 1..491: note = PR007080 Chain
EVQLVESGGGLVQPGGSLRLSCAASGFTFSSYWMHWVRQVPGKGLVWVSRIDSRGSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRIDSRGSRTIYADSVKGRFTISRDNAKNTLYLQMNSLRAEDTAVYYCARGEDHDILTGYPIRGQGTTVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 241 - protein, 491 aa, synthetic construct
  REGION 1..491: note = PR007081 Chain
EVQLVESGGGLVQPGGSLRLSCAASGFTFSSYWMHWVRQVPGKGLVWVSRINFDSSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRINFDSSRTIYADSVKGRFTISRDNAKNTLYLQMNSLRAEDTAVYYCARGEDHDILTGYPIRGQGTTVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 242 - protein, 491 aa, synthetic construct
  REGION 1..491: note = PR007082 Chain
EVQLVESGGGLVQPGGSLRLSCAASGFTFSSYWMHWVRQVPGKGLVWVSRINHDSSRTIYADSVKGRFTISRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVSRINHDSSRTIYADSVKGRFTISRDNAKNTLYLQMNSLRAEDTAVYYCARGEDHDILTGYPIRGQGTTVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 243 - protein, 491 aa, synthetic construct
  REGION 1..491: note = PR007083 Chain
EVQLVESGGGLVQPGGSLRLSCAASGFTFSSYWMHWVRQVPGKGLVWVCRISSDASRTIYADSVKGRFTCSRDNAKNTLYLQMNSLRAEDTAVYYCARGEDHDILTGYPIRGQGTTVTVSSGGGGSGGGGSGGGGSEVQLVESGGGLVQPGGSLRLSCAASGFTFSSYWMHWVRQVPGKGLVWVCRISSDASRTIYADSVKGRFTCSRDNAKNTLYLQMNSLRAEDTAVYYCARGEDHDILTGYPIRGQGTTVTVSSASEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 244 - protein, 5 aa, synthetic construct
  REGION 1..5: note = Linker peptide GS_5
GGGGS

SEQ ID NO: 245 - protein, 10 aa, synthetic construct
  REGION 1..10: note = Linker peptide GS_10
GGGGSGGGGS

SEQ ID NO: 246 - protein, 15 aa, synthetic construct
  REGION 1..15: note = Linker peptide GS_15
GGGGSGGGGSGGGGS

SEQ ID NO: 247 - protein, 20 aa, synthetic construct
  REGION 1..20: note = Linker peptide GS_20
GGGGSGGGGSGGGGSGGGGS

SEQ ID NO: 248 - protein, 25 aa, synthetic construct
  REGION 1..25: note = Linker peptide GS_25
GGGGSGGGGSGGGGSGGGGSGGGGS

SEQ ID NO: 249 - DNA, 24 bp, synthetic construct
  misc_feature 1..24: note = PCR primer-1
  source 1..24: mol_type = other DNA
ggtgtccagtgtsaggtgcagctg

SEQ ID NO: 250 - DNA, 29 bp, synthetic construct
  misc_feature 1..29: note = PCR primer-2
  source 1..29: mol_type = other DNA
aatccctgggcactgaagagacggtgacc

(19) United States
(12) Patent Application Publication
(10) Pub. No.: US 2025/0340631 A1
(43) Pub. Date: Nov. 6, 2025

(54) CLDN18.2-TARGETING ANTIBODY, BISPECIFIC ANTIBODY AND USE THEREOF

(71) Applicant: HARBOUR BioMed (Shanghai) Co., Ltd, SHANGHAI (CN)

(72) Inventors: MING-JIN JHENG, SHANGHAI (CN); YONGQIANG WANG, SHANGHAI (CN); YUN ZHANG, SHANGHAI (CN); CHUCHU ZHAO, SHANGHAI (CN); YUNXING YANG, SHANGHAI (CN); FEI CHEN, SHANGHAI (CN); BEIBEI QIN, SHANGHAI (CN); YUETAO WU, SHANGHAI (CN); YI DING, SHANGHAI (CN)

(21) Appl. No.: 19/273,244

(22) Filed: Jul. 18, 2025

Related U.S. Application Data
(63) Continuation of application No. 17/818,072, filed on Aug. 8, 2022.

(30) Foreign Application Priority Data
Aug. 9, 2021 (CN) 202110909032.3

(51) Int. Cl. C07K 16/28; A61K 39/00; A61K 39/395; A61K 45/06; A61K 47/68; A61P 35/00

(52) U.S. Cl. CPC C07K 16/28; A61K 39/3955; A61K 45/06; A61K 47/6849; A61P 35/00; C07K 16/2809; A61K 2039/505; C07K 2317/14; C07K 2317/24; C07K 2317/31; C07K 2317/51; C07K 2317/515; C07K 2317/52; C07K 2317/522; C07K 2317/524; C07K 2317/526; C07K 2317/53; C07K 2317/55; C07K 2317/56; C07K 2317/565; C07K 2317/567; C07K 2317/569; C07K 2317/622; C07K 2317/73; C07K 2317/734; C07K 2317/92

(57) ABSTRACT

The present invention discloses CLDN18.2-targeting antibodies, bispecific antibodies and use thereof. The CLDN18.2-targeting antibody is a single-domain heavy-chain antibody that has high affinity for tumor cells endogenously expressing CLDN18.2 and can induce high endocytic activity. The bispecific antibody can target CLDN18.2 and CD3 and retains the binding effect of an Fc to an FcRn; meanwhile, a mutant Fc is preferred so as to reduce the binding to an FcgR and thus the activation of non-specific T cells caused by the cross-linking of an FcgR. The CD3-terminus activity is optimized so that the release of common cytokines in CRS, such as IL6 and TNFα can be reduced.

### CROSS-REFERENCE TO RELATED APPLICATION

This application incorporates by reference in its entirety and is a continuation application of pending U.S. Ser. No. 17/818,072 filed on Aug. 8, 2022, which claims benefit under 35 U.S.C. § 119 (b) of Chinese Provisional Application No. 202110909032.3 filed Aug. 9, 2021.

### REFERENCE TO THE SEQUENCE LISTING

This application incorporates by reference a Sequence Listing submitted with this application as text file entitled “2022 Aug. 8-CLDN18T-100_Sequence_Listing_NEW.xml” created on Aug. 8, 2022 and having a size of 348,484 bytes.

### TECHNICAL FIELD

The present invention relates to the field of biomedicine, and particularly to a CLDN18.2-targeting antibody, a bispecific antibody and use thereof.

### BACKGROUND

Cancer is one of the deadliest diseases in humans today. According to the World Health Organization (WHO) Report 2018, there are about 18.07 million new cases of cancer each year. Approximately 9.55 million people each year die of cancer. According to WHO estimates, gastric cancer is ranked fifth among the most commonly diagnosed cancers in the world. Gastric cancer is ranked third (for men) and fourth (for women) among the causes of cancer-related deaths. There are one million new cases of gastric cancer worldwide each year. Approximately 35% of patients who are primarily diagnosed with gastric cancer in the U.S. are those with metastatic gastric cancer. The five-year survival rate for those diagnosed with advanced gastric cancer is 5%, and the median survival is about 6 months. First-line medication for treating patients with metastatic/recurrent gastric cancer is divided into two cases: (1) for HER2-neu positive patients, Transtuzumab is employed in combination with chemotherapeutic drugs; (2) for HER2-neu negative patients, the treatment is limited to chemotherapeutic drugs; however, the treatment outcome is not good (Front Pharmacol. 2018 Sep. 13; 9:404).

The splice variant 1 (CLD18A1, namely CLDN18.1, under the Genbank accession number NP_057453, NM016369) and the splice variant 2 (CLD18A2, namely CLDN18.2, under the Genbank accession number NM_001002026, NP_001002026) of the CLDN18 (Claudin18) molecule are integral transmembrane proteins having a molecular weight of approximately 27.9/27.72 kD. Claudins are integral membrane proteins located in the tight junction of an epithelium and endothelium. The other two major proteins of the tight-junction family are occludin and the junctional adhesion molecule (JAM). Claudins are essential components of the tight junctions, and play an important role in maintaining the polarity of epithelial cells, controlling the paracellular diffusion, and regulating the growth and differentiation of cells. It is speculated that claudins can hardly get near antibodies in well-constructed epithelia but become exposed in tumor cells. The claudin molecule crosses a cell membrane four times, with both the N- and C-termini in the cytoplasm. The human CLDN18.2 (Claudin 18.2) protein is a transmembrane protein having 261 amino acids in full length, among which 1-23 forms a signal peptide; it has two extramembranous regions following the signal peptide, extracellular loop 1 (ECL1) of about 55 amino acids and ECL2 of about 23 amino acids. CLDN18.1 (Claudin 18.1) and CLDN18.2 differ in the first 21 amino acids of the N-terminus including the first TM and loop 1 (i.e., ECL1) but have identical primary protein sequences at the C-terminus. The ECL1 regions of human CLDN18.2 and human CLDN18.1 are very similar, and the ECL2 regions of human CLDN18.2 and human CLDN18.1 are identical. Thus, the development of antibodies for human CLDN18.2 protein targets requires the search for antibodies targeted at the ECL1 region or the spatial structure of the human CLDN18.2 protein. This makes the work in this aspect more difficult. CLDN18.1 is selectively expressed in the epithelium of the normal lungs and stomach (Mol Cell Biol. 2001 November; 21 (21): 7380-90). Expression of CLDN18.2 in normal tissues is highly limited to differentiated cells of the gastric epithelium and absent from the gastric stem cell region. But it is highly expressed in several types of cancer, including gastric, esophageal, pancreatic and lung tumors, as well as human cancer cell lines. The molecular weight of the protein varies in some cancers and adjacent normal tissues. The proteins with a high molecular weight observed in healthy tissues can be converted to those with the same molecular weight as observed in cancer by treating the tissue lysate with the deglycosylating compound PNGaseF. This suggests that claudin is less N-glycosylated in cancer than in its normal tissue counterpart. This structural difference is likely to give rise to an altered epitope. A classical N-glycosylation motif is in the amino acid at position 116 within the loop D3 domain of the molecule. (CN103509110B).

At present, studies on monoclonal antibodies for CLDN18.2 are limited to the phase II and phase III clinical trials of the Claudiximab (IMAB362) antibody (see WO 2014/146672). IMAB362 is capable of inducing ADCC (antibody-dependent cell-mediated cytotoxicity) and CDC (complement dependent cytotoxicity) effects, as well as mediating tumor killing. IMAB362 showed an encouraging effect in the phase I and II clinical trials for the treatment of advanced gastro-esophageal cancer (Eur J Cancer. 2018 September; 100:17-26). However, IMAB362 is a human or murine chimeric antibody and thus involves an immunogenicity risk, and the affinity is not high. Due to the unmet medical need for a large number of malignancies, there is a need for other CLDN18.2 antibodies with more desirable pharmaceutical characteristics. Therefore, there is a lack in the art of effective antibodies targeting the human CLDN18.2 protein, particularly fully human monoclonal antibodies, as well as monoclonal antibodies with better cell-binding activity.

At present, the CLDN18.2×CD3 bispecific antibody under clinical development includes AMG910 of Amgen. AMG910 can induce a TDCC (T-cell-dependent-cellular-cytotoxicity) effect to mediate tumor killing. However, the antibodies in the prior art may have the problems of short half-lives, poor drug effects, causing cytokine release syndrome (CRS), etc. Therefore, there is an urgent need to develop safer and more effective bispecific antibodies that target both human CLDN18.2 and CD3 and can bind to cynomolgus CLDN18.2 and CD3.

### SUMMARY

To solve the technical problems in the prior art that safe and effective monoclonal antibodies targeting human CLDN18.2 and bispecific antibodies that target both human CLDN18.2 and CD3 and can bind to cynomolgus CLDN18.2 and CD3 are lacking, the present invention provides a CLDN18.2-targeting monoclonal antibody, a CLDN18.2 and CD3-targeting bispecific antibody and use thereof.

To solve the above technical problems, a first aspect of the present invention provides a CLDN18.2-targeting antibody comprising a heavy chain variable region comprising HCDR1, HCDR2 and HCDR3, wherein the HCDR1 comprises an amino acid sequence as set forth in any one of SEQ ID NOs: 16-18, the HCDR2 comprises an amino acid sequence as set forth in any one of SEQ ID NOs: 42-46 and SEQ ID NOs: 48-54, and the HCDR3 comprises an amino acid sequence as set forth in any one of SEQ ID NOs: 77-82.

In a preferred embodiment of the present invention, the HCDR1, the HCDR2 and the HCDR3 comprise amino acid sequences as set forth in SEQ ID NO: 16, SEQ ID NO: 42 and SEQ ID NO: 77, respectively; or
the HCDR1, the HCDR2 and the HCDR3 comprise amino acid sequences as set forth in SEQ ID NO: 16, SEQ ID NO: 43 and SEQ ID NO: 78, respectively; or
the HCDR1, the HCDR2 and the HCDR3 comprise amino acid sequences as set forth in SEQ ID NO: 16, SEQ ID NO: 44 and SEQ ID NO: 79, respectively; or
the HCDR1, the HCDR2 and the HCDR3 comprise amino acid sequences as set forth in SEQ ID NO: 17, SEQ ID NO: 45 and SEQ ID NO: 80, respectively; or
the HCDR1, the HCDR2 and the HCDR3 comprise amino acid sequences as set forth in SEQ ID NO: 18, SEQ ID NO: 43 and SEQ ID NO: 80, respectively; or
the HCDR1, the HCDR2 and the HCDR3 comprise amino acid sequences as set forth in SEQ ID NO: 18, SEQ ID NO: 43 and SEQ ID NO: 78, respectively; or
the HCDR1, the HCDR2 and the HCDR3 comprise amino acid sequences as set forth in SEQ ID NO: 16, SEQ ID NO: 43 and SEQ ID NO: 81, respectively; or
the HCDR1, the HCDR2 and the HCDR3 comprise amino acid sequences as set forth in SEQ ID NO: 16, SEQ ID NO: 46 and SEQ ID NO: 82, respectively; or
the HCDR1, the HCDR2 and the HCDR3 comprise amino acid sequences as set forth in SEQ ID NO: 16, SEQ ID NO: 48 and SEQ ID NO: 78, respectively; or
the HCDR1, the HCDR2 and the HCDR3 comprise amino acid sequences as set forth in SEQ ID NO: 16, SEQ ID NO: 49 and SEQ ID NO: 78, respectively; or
the HCDR1, the HCDR2 and the HCDR3 comprise amino acid sequences as set forth in SEQ ID NO: 16, SEQ ID NO: 50 and SEQ ID NO: 78, respectively; or
the HCDR1, the HCDR2 and the HCDR3 comprise amino acid sequences as set forth in SEQ ID NO: 16, SEQ ID NO: 51 and SEQ ID NO: 78, respectively; or
the HCDR1, the HCDR2 and the HCDR3 comprise amino acid sequences as set forth in SEQ ID NO: 16, SEQ ID NO: 52 and SEQ ID NO: 78, respectively; or
the HCDR1, the HCDR2 and the HCDR3 comprise amino acid sequences as set forth in SEQ ID NO: 16, SEQ ID NO: 53 and SEQ ID NO: 79, respectively; or
the HCDR1, the HCDR2 and the HCDR3 comprise amino acid sequences as set forth in SEQ ID NO: 16, SEQ ID NO: 54 and SEQ ID NO: 78, respectively.

The above combinations of amino acid sequences for the HCDR1, the HCDR2 and the HCDR3 are detailed in Table a below.

TABLE a
The HCDR combination of the CLDN18.2-
targeting antibody

| | | HCDR1 | | HCDR2 | | HCDR3 | |
|---|---|---|---|---|---|---|---|
| | | | Corre- | | Corre- | | Corre- |
| | HCDRs | | spond- | | spond- | | spond- |
| | Anti- | SEQ | ing | SEQ | ing | SEQ | ing |
| | body | ID | se- | ID | se- | ID | se- |
| No. | No. | NOs: | quences | NOs: | quences | NOs: | quences |
| 1 | PR004227 | 16 | GFTFSSY | 42 | NNDGSS | 77 | APPYG |
| | | | | | | | NYERD |
| | | | | | | | Y |
| 2 | PR004533 | 16 | GFTFSSY | 43 | NSDGSR | 78 | GEDHD |
| | | | | | | | ILTGY |
| | | | | | | | PI |
| 3 | PR004536 | 16 | GFTFSSY | 44 | NSDGST | 79 | DFPVL |
| | | | | | | | GGSHF |
| | | | | | | | DFQH |
| 4 | PR004540 | 17 | GFTFSIY | 45 | SSDGNY | 80 | GEDHD |
| | | | | | | | MLTGY |
| | | | | | | | PV |
| 5 | PR004949 | 18 | GFTFSAY | 43 | NSDGSR | 80 | GEDHD |
| | | | | | | | MLTGY |
| | | | | | | | PV |
| 6 | PR004950 | 18 | GFTFSAY | 43 | NSDGSR | 78 | GEDHD |
| | | | | | | | ILTGY |
| | | | | | | | PI |
| 7 | PR004952 | 16 | GFTFSSY | 43 | NSDGSR | 81 | GEDHD |
| | | | | | | | ILTGY |
| | | | | | | | PV |
| 8 | PR004953 | 16 | GFTFSSY | 46 | GYDGRN | 82 | HFPSL |
| | | | | | | | PGTTD |
| | | | | | | | TFDI |
| 9 | PR007242 | 16 | GFTFSSY | 48 | NFDSSR | 78 | GEDHD |
| | | | | | | | ILTGY |
| | | | | | | | PI |
| 10 | PR007243 | 16 | GFTFSSY | 49 | SSAGSR | 78 | GEDHD |
| | | | | | | | ILTGY |
| | | | | | | | PI |
| 11 | PR007244 | 16 | GFTFSSY | 50 | DSRGSR | 78 | GEDHD |
| | | | | | | | ILTGY |
| | | | | | | | PI |
| 12 | PR007245 | 16 | GFTFSSY | 51 | SSDASR | 78 | GEDHD |
| | | | | | | | ILTGY |
| | | | | | | | PI |
| 13 | PR007246 | 16 | GFTFSSY | 52 | SSTGSR | 78 | GEDHD |
| | | | | | | | ILTGY |
| | | | | | | | PI |
| 14 | PR007247 | 16 | GFTFSSY | 53 | NPLGST | 79 | DFPVL |
| | | | | | | | GGSHF |
| | | | | | | | DFQH |
| 15 | PR007248 | 16 | GFTFSSY | 54 | NHDSSR | 78 | GEDHD |
| | | | | | | | ILTGY |
| | | | | | | | PI |

In a preferred embodiment of the present invention, the heavy chain variable region further comprises framework regions, among which the HFR1 comprises an amino acid sequence as set forth in SEQ ID NO: 6 or 7, the HFR2 comprises an amino acid sequence as set forth in any one of SEQ ID NOs: 28-34, the HFR3 comprises an amino acid sequence as set forth in any one of SEQ ID NOs: 63-68, and the HFR4 comprises an amino acid sequence as set forth in any one of SEQ ID NOs: 84 and 86-89.

In a preferred embodiment of the present invention, the heavy chain variable region comprises an amino acid sequence as set forth in any one of SEQ ID NOs: 150-157 and SEQ ID NOs: 159-165. See Table h below for details.

TABLE b
The heavy chain variable region of
the CLDN18.2-targeting antibody

| SEQ | |
|---|---|
| ID | |
| NOs: | Corresponding sequences |
| 150 | EVQLVESGGGLVQPGGSLRLSCAASGFTFSS |
| | YWMHWVRQAPGKGLEWVSYINNDGSSTRYA |
| | DSVKGRFTISRDNAKNTLYLQMNSLRAEDTA |
| | VYYCTEAPPYGNYERDYWGQGTLVTVSS |
| 151 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSS |
| 152 | EVQLVESGGGLVQPGGSLRLSCAASGFTFSS |
| | YWMYWVRQVPGKGLVWVSHINSDGSTTQYA |
| | DSVKGRFTISRDNAKNMLYLQMNSLRAEDTA |
| | VYYCARDFPVLGGSHFDFQHWGQGTLVTV |
| | SS |
| 153 | EVQLVESGGGLVQPGGSLRLSCAASGFTFSI |
| | YWMHWVRQVPGKGLVLVSRISSDGNYTSYA |
| | DSVKGRFTISRDNAKNTLYLQMNSLRAEDTA |
| | VYLCARGEDHDMLTGYPVRGQGTTVTVSS |
| 154 | EVQLVESGGGLVQPGGSLRLSCAASGFTFSA |
| | YWMHWVRQVPGRGLVWVSRINSDGSRTIYA |
| | DSVKGRFTISRDNAKNTLYLQMNSLRAEDTA |
| | VYLCARGEDHDMLTGYPVRGQGTMVTVSS |
| 155 | EVQLVESGGGLVQPGGSLRLSCAASGFTFSA |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTMVTVSS |
| 156 | EVQLVESGGGLVQPGGSLRLSCAASGFTFSS |
| | YWMHWVRQAPGKGLVWVSRINSDGSRTIYA |
| | DSVKGRFTISRDNAKNTLYLQMNSLRAEDTA |
| | VYYCARGEDHDILTGYPVRGQGATVTVSS |
| 157 | EVQLVESGGGVVQPGRSLRLSCAASGFTFSS |
| | YGMHWVRQAPGKGLEWVAVIGYDGRNKYY |
| | ADSVKGRFTISRDNSKNTLFLQMDNLRAEDT |
| | ALYYCARHFPSLPGTTDTFDIRGPGTMVTVS |
| | S |
| 159 | EVQLVESGGGLVQPGGSLRLSCAASGFTFSS |
| | YWMHWVRQVPGKGLVWVSRINFDSSRTIYA |
| | DSVKGRFTISRDNAKNTLYLQMNSLRAEDTA |
| | VYYCARGEDHDILTGYPIRGQGTTVTVSS |
| 160 | EVQLVESGGGLVQPGGSLRLSCAASGFTFSS |
| | YWMHWVRQVPGKGLVWVSRISSAGSRTIYA |
| | DSVKGRFTISRDNAKNTLYLQMNSLRAEDTA |
| | VYYCARGEDHDILTGYPIRGQGTTVTVSS |
| 161 | EVQLVESGGGLVQPGGSLRLSCAASGFTFSS |
| | YWMHWVRQVPGKGLVWVSRIDSRGSRTIYA |
| | DSVKGRFTISRDNAKNTLYLQMNSLRAEDTA |
| | VYYCARGEDHDILTGYPIRGQGTTVTVSS |
| 162 | EVQLVESGGGLVQPGGSLRLSCAASGFTFSS |
| | YWMHWVRQVPGKGLVWVSRISSDASRTIYA |
| | DSVKGRFTISRDNAKNTLYLQMNSLRAEDTA |
| | VYYCARGEDHDILTGYPIRGQGTTVTVSS |
| 163 | EVQLVESGGGLVQPGGSLRLSCAASGFTFSS |
| | YWMHWVRQVPGKGLVWVSRISSTGSRTIYA |
| | DSVKGRFTISRDNAKNTLYLQMNSLRAEDTA |
| | VYYCARGEDHDILTGYPIRGQGTTVTVSS |
| 164 | EVQLVESGGGLVQPGGSLRLSCAASGFTFSS |
| | YWMYWVRQVPGKGLVWVSHINPLGSTTQYA |
| | DSVKGRFTISRDNAKNMLYLQMNSLRAEDTA |
| | VYYCARDFPVLGGSHFDFQHWGQGTLVTV |
| | SS |
| 165 | EVQLVESGGGLVQPGGSLRLSCAASGFTFSS |
| | YWMHWVRQVPGKGLVWVSRINHDSSRTIYA |
| | DSVKGRFTISRDNAKNTLYLQMNSLRAEDTA |
| | VYYCARGEDHDILTGYPIRGQGTTVTVSS |

In a preferred embodiment of the present invention, the antibody further comprises a heavy chain constant region. More preferably, the heavy chain constant region is selected from hIgG1, hIgG2, hIgG3 and hIgG4 and a variant thereof. Even more preferably, the heavy chain constant region is hIgG1.

In a preferred embodiment of the present invention, the antibody is a full-length antibody, an Fab, an Fab′, an F(ab′)₂, an Fv, an scFv, a bispecific antibody, a multispecific antibody, a heavy-chain antibody or a single-domain antibody, or a monoclonal or polyclonal antibody prepared from the antibodies above.

In a more preferred embodiment of the present invention, the antibody is a single-domain antibody comprising an amino acid sequence as set forth in any one of SEQ ID NOs: 182-189 and SEQ ID NOs: 191-197. See Table c below for details.

TABLE c
The heavy chain of the CLDN18.2-
targeting antibody

| SEQ | |
|---|---|
| ID | |
| NOs: | Corresponding sequences |
| 182 | EVQLVESGGGLVQPGGSLRLSCAASGFTFSS |
| | YWMHWVRQAPGKGLEWVSYINNDGSSTRYA |
| | DSVKGRFTISRDNAKNTLYLQMNSLRAEDTA |
| | VYYCTEAPPYGNYERDYWGQGTLVTVSSGQ |
| | AGQEPKSSDKTHTCPPCPAPELLGGPSVFLF |
| | PPKPKDTLMISRTPEVTCVVVDVSHEDPEVK |
| | FN |
| | WYVDGVEVHNAKTKPREEQYNSTYRVVSVLT |
| | VLHQDWLNGKEYKCKVSNKALPAPIEKTIS |
| | KAKGQPREPQVYTLPPSREEMTKNQVSLTCL |
| | VKGFYPSDIAVEWESNGQPENNYKTTPPVLD |
| | S |
| | DGSFFLYSKLTVDKSRWQQGNVFSCSVMHEA |
| | LHNHYTQKSLSLSPGK |
| 183 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSEP |
| | K |
| | SSDKTHTCPPCPAPELLGGPSVFLFPPKPKD |
| | TLMISRTPEVTCVVVDVSHEDPEVKFNWYVD |
| | G |
| | VEVHNAKTKPREEQYNSTYRVVSVLTVLHQD |
| | WLNGKEYKCKVSNKALPAPIEKTISKAKGQP |
| | REPQVYTLPPSREEMTKNQVSLTCLVKGFYP |
| | SDIAVEWESNGQPENNYKTTPPVLDSDGSFF |
| | L |
| | YSKLTVDKSRWQQGNVFSCSVMHEALHNHYT |
| | QKSLSLSPGK |
| 184 | EVQLVESGGGLVQPGGSLRLSCAASGFTFSS |
| | YWMYWVRQVPGKGLVWVSHINSDGSTTQYA |
| | DSVKGRFTISRDNAKNMLYLQMNSLRAEDTA |
| | VYYCARDFPVLGGSHFDFQHWGQGTLVTVS |
| | SEPKSSDKTHTCPPCPAPELLGGPSVFLFPP |
| | KPKDTLMISRTPEVTCVVVDVSHEDPEVKFN |
| | WY |
| | VDGVEVHNAKTKPREEQYNSTYRVVSVLTVL |
| | HQDWLNGKEYKCKVSNKALPAPIEKTISKA |
| | KGQPREPQVYTLPPSREEMTKNQVSLTCLVK |
| | GFYPSDIAVEWESNGQPENNYKTTPPVLDSD |
| | G |
| | SFFLYSKLTVDKSRWQQGNVFSCSVMHEALH |
| | NHYTQKSLSLSPGK |
| 185 | EVQLVESGGGLVQPGGSLRLSCAASGFTFSI |
| | YWMHWVRQVPGKGLVLVSRISSDGNYTSYAD |
| | SVKGRFTISRDNAKNTLYLQMNSLRAEDTAV |
| | YLCARGEDHDMLTGYPVRGQGTTVTVSSEP |
| | KSSDKTHTCPPCPAPELLGGPSVFLFPPKPK |
| | DTLMISRTPEVTCVVVDVSHEDPEVKFNWYV |
| | D |
| | GVEVHNAKTKPREEQYNSTYRVVSVLTVLHQ |
| | DWLNGKEYKCKVSNKALPAPIEKTISKAKG |
| | QPREPQVYTLPPSREEMTKNQVSLTCLVKGF |
| | YPSDIAVEWESNGQPENNYKTTPPVLDSDGS |
| | F |
| | FLYSKLTVDKSRWQQGNVFSCSVMHEALHNH |
| | YTQKSLSLSPGK |
| 186 | EVQLVESGGGLVQPGGSLRLSCAASGFTFSA |
| | YWMHWVRQVPGRGLVWVSRINSDGSRTIYA |
| | DSVKGRFTISRDNAKNTLYLQMNSLRAEDTA |
| | VYLCARGEDHDMLTGYPVRGQGTMVTVSSE |
| | PKSSDKTHTCPPCPAPELLGGPSVFLFPPKP |
| | KDTLMISRTPEVTCVVVDVSHEDPEVKFNWY |
| | V |
| | DGVEVHNAKTKPREEQYNSTYRVVSVLTVLH |
| | QDWLNGKEYKCKVSNKALPAPIEKTISKAK |
| | GQPREPQVYTLPPSREEMTKNQVSLTCLVKG |
| | FYPSDIAVEWESNGQPENNYKTTPPVLDSDG |
| | S |
| | FFLYSKLTVDKSRWQQGNVFSCSVMHEALHN |
| | HYTQKSLSLSPGK |
| 187 | EVQLVESGGGLVQPGGSLRLSCAASGFTFSA |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTMVTVSSEP |
| | KSSDKTHTCPPCPAPELLGGPSVFLFPPKPK |
| | DTLMISRTPEVTCVVVDVSHEDPEVKFNWYV |
| | D |
| | GVEVHNAKTKPREEQYNSTYRVVSVLTVLHQ |
| | DWLNGKEYKCKVSNKALPAPIEKTISKAKG |
| | QPREPQVYTLPPSREEMTKNQVSLTCLVKGF |
| | YPSDIAVEWESNGQPENNYKTTPPVLDSDGS |
| | F |
| | FLYSKLTVDKSRWQQGNVFSCSVMHEALHNH |
| | YTQKSLSLSPGK |
| 188 | EVQLVESGGGLVQPGGSLRLSCAASGFTFSS |
| | YWMHWVRQAPGKGLVWVSRINSDGSRTIYA |
| | DSVKGRFTISRDNAKNTLYLQMNSLRAEDTA |
| | VYYCARGEDHDILTGYPVRGQGATVTVSSEP |
| | KSSDKTHTCPPCPAPELLGGPSVFLFPPKPK |
| | DTLMISRTPEVTCVVVDVSHEDPEVKFNWYV |
| | D |
| | GVEVHNAKTKPREEQYNSTYRVVSVLTVLHQ |
| | DWLNGKEYKCKVSNKALPAPIEKTISKAKG |
| | QPREPQVYTLPPSREEMTKNQVSLTCLVKGF |
| | YPSDIAVEWESNGQPENNYKTTPPVLDSDGS |
| | F |
| | FLYSKLTVDKSRWQQGNVFSCSVMHEALHNH |
| | YTQKSLSLSPGK |
| 189 | EVQLVESGGGVVQPGRSLRLSCAASGFTFSS |
| | YGMHWVRQAPGKGLEWVAVIGYDGRNKYY |
| | ADSVKGRFTISRDNSKNTLFLQMDNLRAEDT |
| | ALYYCARHFPSLPGTTDTFDIRGPGTMVTVS |
| | S |
| | EPKSSDKTHTCPPCPAPELLGGPSVFLFPPK |
| | PKDTLMISRTPEVTCVVVDVSHEDPEVKFNW |
| | YV |
| | DGVEVHNAKTKPREEQYNSTYRVVSVLTVLH |
| | QDWLNGKEYKCKVSNKALPAPIEKTISKAK |
| | GQPREPQVYTLPPSREEMTKNQVSLTCLVKG |
| | FYPSDIAVEWESNGQPENNYKTTPPVLDSDG |
| | S |
| | FFLYSKLTVDKSRWQQGNVFSCSVMHEALHN |
| | HYTQKSLSLSPGK |
| 191 | EVQLVESGGGLVQPGGSLRLSCAASGFTFSS |
| | YWMHWVRQVPGKGLVWVSRINFDSSRTIYAD |
| | SVKGRFTISRDNAKNTLYLQMNSLRAEDTAV |
| | YYCARGEDHDILTGYPIRGQGTTVTVSSEPK |
| | S |
| | SDKTHTCPPCPAPELLGGPSVFLFPPKPKDT |
| | LMISRTPEVTCVVVDVSHEDPEVKFNWYVDG |
| | V |
| | EVHNAKTKPREEQYNSTYRVVSVLTVLHQDW |
| | LNGKEYKCKVSNKALPAPIEKTISKAKGQPR |
| | EPQVYTLPPSREEMTKNQVSLTCLVKGFYPS |
| | DIAVEWESNGQPENNYKTTPPVLDSDGSFFL |
| | Y |
| | SKLTVDKSRWQQGNVFSCSVMHEALHNHYTQ |
| | KSLSLSPGK |
| 192 | EVQLVESGGGLVQPGGSLRLSCAASGFTFSS |
| | YWMHWVRQVPGKGLVWVSRISSAGSRTIYAD |
| | SVKGRFTISRDNAKNTLYLQMNSLRAEDTAV |
| | YYCARGEDHDILTGYPIRGQGTTVTVSSEPK |
| | S |
| | SDKTHTCPPCPAPELLGGPSVFLFPPKPKDT |
| | LMISRTPEVTCVVVDVSHEDPEVKFNWYVDG |
| | V |
| | EVHNAKTKPREEQYNSTYRVVSVLTVLHQDW |
| | LNGKEYKCKVSNKALPAPIEKTISKAKGQPR |
| | EPQVYTLPPSREEMTKNQVSLTCLVKGFYPS |
| | DIAVEWESNGQPENNYKTTPPVLDSDGSFFL |
| | Y |
| | SKLTVDKSRWQQGNVFSCSVMHEALHNHYTQ |
| | KSLSLSPGK |
| 193 | EVQLVESGGGLVQPGGSLRLSCAASGFTFSS |
| | YWMHWVRQVPGKGLVWVSRIDSRGSRTIYAD |
| | SVKGRFTISRDNAKNTLYLQMNSLRAEDTAV |
| | YYCARGEDHDILTGYPIRGQGTTVTVSSEPK |
| | S |
| | SDKTHTCPPCPAPELLGGPSVFLFPPKPKDT |
| | LMISRTPEVTCVVVDVSHEDPEVKFNWYVDG |
| | V |
| | EVHNAKTKPREEQYNSTYRVVSVLTVLHQDW |
| | LNGKEYKCKVSNKALPAPIEKTISKAKGQPR |
| | EPQVYTLPPSREEMTKNQVSLTCLVKGFYPS |
| | DIAVEWESNGQPENNYKTTPPVLDSDGSFFL |
| | Y |
| | SKLTVDKSRWQQGNVFSCSVMHEALHNHYTQ |
| | KSLSLSPGK |
| 194 | EVQLVESGGGLVQPGGSLRLSCAASGFTFSS |
| | YWMHWVRQVPGKGLVWVSRISSDASRTIYAD |
| | SVKGRFTISRDNAKNTLYLQMNSLRAEDTAV |
| | YYCARGEDHDILTGYPIRGQGTTVTVSSEPK |
| | S |
| | SDKTHTCPPCPAPELLGGPSVFLFPPKPKDT |
| | LMISRTPEVTCVVVDVSHEDPEVKFNWYVDG |
| | V |
| | EVHNAKTKPREEQYNSTYRVVSVLTVLHQDW |
| | LNGKEYKCKVSNKALPAPIEKTISKAKGQPR |
| | EPQVYTLPPSREEMTKNQVSLTCLVKGFYPS |
| | DIAVEWESNGQPENNYKTTPPVLDSDGSFFL |
| | Y |
| | SKLTVDKSRWQQGNVFSCSVMHEALHNHYTQ |
| | KSLSLSPGK |
| 195 | EVQLVESGGGLVQPGGSLRLSCAASGFTFSS |
| | YWMHWVRQVPGKGLVWVSRISSTGSRTIYAD |
| | SVKGRFTISRDNAKNTLYLQMNSLRAEDTAV |
| | YYCARGEDHDILTGYPIRGQGTTVTVSSEPK |
| | S |
| | SDKTHTCPPCPAPELLGGPSVFLFPPKPKDT |
| | LMISRTPEVTCVVVDVSHEDPEVKFNWYVDG |
| | V |
| | EVHNAKTKPREEQYNSTYRVVSVLTVLHQDW |
| | LNGKEYKCKVSNKALPAPIEKTISKAKGQPR |
| | EPQVYTLPPSREEMTKNQVSLTCLVKGFYPS |
| | DIAVEWESNGQPENNYKTTPPVLDSDGSFFL |
| | Y |
| | SKLTVDKSRWQQGNVFSCSVMHEALHNHYTQ |
| | KSLSLSPGK |
| 196 | EVQLVESGGGLVQPGGSLRLSCAASGFTFSS |
| | YWMYWVRQVPGKGLVWVSHINPLGSTTQYA |
| | DSVKGRFTISRDNAKNMLYLQMNSLRAEDTA |
| | VYYCARDFPVLGGSHFDFQHWGQGTLVTVS |
| | SEPKSSDKTHTCPPCPAPELLGGPSVFLFPP |
| | KPKDTLMISRTPEVTCVVVDVSHEDPEVKFN |
| | WY |
| | VDGVEVHNAKTKPREEQYNSTYRVVSVLTVL |
| | HQDWLNGKEYKCKVSNKALPAPIEKTISKA |
| | KGQPREPQVYTLPPSREEMTKNQVSLTCLVK |
| | GFYPSDIAVEWESNGQPENNYKTTPPVLDSD |
| | G |
| | SFFLYSKLTVDKSRWQQGNVFSCSVMHEALH |
| | NHYTQKSLSLSPGK |
| 197 | EVQLVESGGGLVQPGGSLRLSCAASGFTFSS |
| | YWMHWVRQVPGKGLVWVSRINHDSSRTIYA |
| | DSVKGRFTISRDNAKNTLYLQMNSLRAEDTA |
| | VYYCARGEDHDILTGYPIRGQGTTVTVSSEP |
| | K |
| | SSDKTHTCPPCPAPELLGGPSVFLFPPKPKD |
| | TLMISRTPEVTCVVVDVSHEDPEVKFNWYVD |
| | G |
| | VEVHNAKTKPREEQYNSTYRVVSVLTVLHQD |
| | WLNGKEYKCKVSNKALPAPIEKTISKAKGQP |
| | REPQVYTLPPSREEMTKNQVSLTCLVKGFYP |
| | SDIAVEWESNGQPENNYKTTPPVLDSDGSFF |
| | L |
| | YSKLTVDKSRWQQGNVFSCSVMHEALHNHYT |
| | QKSLSLSPGK |

In the present invention, an “Fab fragment” consists of one light chain and CH1 and the variable region of one heavy chain. The heavy chain of an Fab molecule cannot form disulfide bonds with another heavy chain molecule. An “Fc” region contains two heavy chain fragments comprising the CH1 and CH2 domains of an antibody. The two heavy chain fragments are held together by two or more disulfide bonds and the hydrophobic interaction of the CH3 domains. An “Fab′ fragment” contains one light chain and part of one heavy chain comprising the VH domain and the CH1 domain and the region between the CH1 and CH2 domains, so that interchain disulfide bonds can be formed between the two heavy chains of two Fab′ fragments to provide an F(ab′)₂ molecule. An “F(ab′)₂ fragment” contains two light chains and two heavy chains comprising part of the constant region between the CH1 and CH2 domains, such that interchain disulfide bonds are formed between the two heavy chains. Thus, an F(ab′)₂ fragment consists of two Fab′ fragments held together by disulfide bonds between the two heavy chains. The term “Fv” refers to an antibody fragment consisting of the VL and VH domains of a single arm of an antibody, but lacks the constant region.

In the present invention, the scFv (single chain antibody fragment) may be a conventional single chain antibody in the art, which comprises a heavy chain variable region, a light chain variable region, and a short peptide of 15-20 amino acids. In the scFv, the VL and VH domains are paired to form a monovalent molecule via a linker that enables them to produce a single polypeptide chain [see, e.g., Bird et al, Science 242:423-426 (1988) and Huston et al, Proc. Natl. Acad. Sci. USA 85:5879-5883 (1988)]. Such scFv molecules may have a general structure: NH2-VL-linker-VH—COOH or NH2-VH-linker-VL-COOH. An appropriate linker in the prior art consists of repeated G4S amino acid sequences or a variant thereof. For example, linkers having the amino acid sequence (G4S) 4 or (G4S) 3 may be used, but a variant thereof may also be used.

The term “multispecific antibody” is used in its widest sense to encompass antibodies having multi-epitope specificity. These multispecific antibodies include, but are not limited to: an antibody comprising a heavy chain variable region (VH) and a light chain variable region (VL), the VH-VL unit having multi-epitope specificity; an antibody having two or more VL and VH regions, each VH-VL unit binding to a different target or a different epitope of the same target; an antibody having two or more single variable regions, each single variable region binding to a different target or a different epitope of the same target; full length antibodies, antibody fragments, bispecific antibodies, triabodies, antibody fragments linked together covalently or non-covalently, and the like.

The antibody of the present invention includes a monoclonal antibody. The monoclonal antibody or mAb or Ab of the present invention refers to an antibody obtained from a single clonal cell line, which is not limited to eukaryotic, prokaryotic, or phage clonal cell lines.

In the present invention, the “heavy-chain antibody”, also referred to as HCAbs, refers to an antibody comprising only one heavy chain variable region (VHH) and two conventional CH2 and CH3 regions.

In the present invention, the “single-domain antibody”, also referred to as “nanobody”, refers to a VHH structure cloned from a heavy-chain antibody. It is the smallest unit known to be able to bind to a target antigen.

To solve the above technical problems, a second aspect of the present invention provides a bispecific antibody comprising a first protein functional region targeting CD3 and a second protein functional region targeting CLDN18.2;

the first protein functional region is in the form of an Fab, and the second protein functional region is in the form of VHs and preferably comprises 2 or 3 VHs; when the second protein functional region comprises 3 VHs linked in series, the first protein functional region and the second protein functional region are each linked to an Fc's double strand; when the second protein functional region comprises 2 VHs linked in series, the first protein functional region and the second protein functional region are each linked to an Fc's double strand; when the second protein functional region comprises 3 VHs and one of the 3 VHs is linked to the first protein functional region, the remaining two VHs are linked in series, and the first protein functional region and the two VHs linked in series of the second protein functional region are each linked to an Fc's double strand;

alternatively, the first protein functional region is in the form of an Fab and the second protein functional region is in the form of an HCAb;
alternatively, the first protein functional region is in the form of an Fab and the second protein functional region is in the form of a VH-HCAb, the second protein functional region preferably comprising a total of 4 VHs.

In the present invention, the “first” and “second” in the first protein functional region and the second protein functional region have no practical meaning, and are only used to distinguish antigen-binding domains for different targets. One protein functional region may comprise a plurality of antigen-binding domains in the same form or different forms; the antigen-binding domains of different protein functional regions may be operably linked together, and different antigen-binding domains of the same protein functional region may not be linked to each other.

For example, in the present invention, the first protein functional region may be a CD3-targeting antigen-binding domain, and the second protein functional region may be a CLDN18.2-targeting antigen-binding domain.

In a preferred embodiment of the present invention, the second protein functional region comprises a heavy chain variable region comprising HCDR1, HCDR2 and HCDR3, wherein the HCDR1 comprises an amino acid sequence as set forth in any one of SEQ ID NOs: 16-18, the HCDR2 comprises an amino acid sequence as set forth in any one of SEQ ID NOs: 42-46 and SEQ ID NOs: 48-54, and the HCDR3 comprises an amino acid sequence as set forth in any one of SEQ ID NOs: 77-82.

More preferably, the HCDR1, the HCDR2 and the HCDR3 comprise amino acid sequences as set forth in SEQ ID NO: 16, SEQ ID NO: 42 and SEQ ID NO: 77, respectively; or the HCDR1, the HCDR2 and the HCDR3 comprise amino acid sequences as set forth in SEQ ID NO: 16, SEQ ID NO: 43 and SEQ ID NO: 78, respectively; or the HCDR1, the HCDR2 and the HCDR3 comprise amino acid sequences as set forth in SEQ ID NO: 16, SEQ ID NO: 44 and SEQ ID NO: 79, respectively; or the HCDR1, the HCDR2 and the HCDR3 comprise amino acid sequences as set forth in SEQ ID NO: 16, SEQ ID NO: 48 and SEQ ID NO: 78, respectively; or the HCDR1, the HCDR2 and the HCDR3 comprise amino acid sequences as set forth in SEQ ID NO: 16, SEQ ID NO: 49 and SEQ ID NO: 78, respectively; or the HCDR1, the HCDR2 and the HCDR3 comprise amino acid sequences as set forth in SEQ ID NO: 16, SEQ ID NO: 50 and SEQ ID NO: 78, respectively; or the HCDR1, the HCDR2 and the HCDR3 comprise amino acid sequences as set forth in SEQ ID NO: 16, SEQ ID NO: 51 and SEQ ID NO: 78, respectively; or the HCDR1, the HCDR2 and the HCDR3 comprise amino acid sequences as set forth in SEQ ID NO: 16, SEQ ID NO: 52 and SEQ ID NO: 78, respectively; or the HCDR1, the HCDR2 and the HCDR3 comprise amino acid sequences as set forth in SEQ ID NO: 16, SEQ ID NO: 53 and SEQ ID NO: 79, respectively; or the HCDR1, the HCDR2 and the HCDR3 comprise amino acid sequences as set forth in SEQ ID NO: 16, SEQ ID NO: 54 and SEQ ID NO: 78, respectively. See Table d below for details.

TABLE d
The HCDR combination of the second
protein functional region of the
bispecific antibody

| | | HCDR1 | | HCDR2 | | HCDR3 | |
|---|---|---|---|---|---|---|---|
| | | | Corre- | | Corre- | | Corre- |
| | | | spond- | | spond- | | spond- |
| | HCDRs | SEQ | ing | SEQ | ing | SEQ | ing |
| | Antibody | ID | se- | ID | se- | ID | se- |
| No. | No. | NOs: | quences | NOs: | quences | NOs: | quences |
| 1 | PR004603 | 16 | GFTFSSY | 42 | NNDGSS | 77 | APPYG |
| | | | | | | | NYERD |
| | | | | | | | Y |
| 2 | PR005072, | 16 | GFTFSSY | 43 | NSDGSR | 78 | GEDHD |
| | PR005354, | | | | | | ILTGY |
| | PR005518, | | | | | | PI |
| | PR005519, | | | | | | |
| | PR005520, | | | | | | |
| | PR005521, | | | | | | |
| | PR005522, | | | | | | |
| | PR005525, | | | | | | |
| 3 | PR005076 | 16 | GFTFSSY | 44 | NSDGST | 79 | DFPVL |
| | | | | | | | GGSHF |
| | | | | | | | DFQH |
| 4 | PR005397, | 16 | GFTFSSY | 52 | SSTGSR | 78 | GEDHD |
| | PR006384, | | | | | | ILTGY |
| | PR006023 | | | | | | PI |
| 5 | PR005398, | 16 | GFTFSSY | 48 | NFDSSR | 78 | GEDHD |
| | PR007081 | | | | | | ILTGY |
| | | | | | | | PI |
| 6 | PR005399, | 16 | GFTFSSY | 49 | SSAGSR | 78 | GEDHD |
| | PR007079 | | | | | | ILTGY |
| | | | | | | | PI |
| 7 | PR005401, | 16 | GFTFSSY | 50 | DSRGSR | 78 | GEDHD |
| | PR007080 | | | | | | ILTGY |
| | | | | | | | PI |
| 8 | PR005411, | 16 | GFTFSSY | 51 | SSDASR | 78 | GEDHD |
| | PR006292, | | | | | | ILTGY |
| | PR007083, | | | | | | PI |
| | PR006293 | | | | | | |
| 9 | PR005422 | 16 | GFTFSSY | 53 | NPLGST | 79 | DFPVL |
| | | | | | | | GGSHF |
| | | | | | | | DFQH |
| 10 | PR007082 | 16 | GFTFSSY | 54 | NHDSSR | 78 | GEDHD |
| | | | | | | | ILTGY |
| | | | | | | | PI |

Even more preferably, the heavy chain variable region comprises an amino acid sequence as set forth in any one of SEQ ID NOs: 150-152 and SEQ ID NOs: 159-165, all of which have been listed in Table b.

In a specific embodiment of the present invention, the first protein functional region comprises a light chain variable region comprising LCDR1, LCDR2 and LCDR3 as set forth in SEQ ID NO: 101, SEQ ID NO: 116 and SEQ ID NO: 131, respectively, and a heavy chain variable region comprising HCDR1, HCDR2 and HCDR3 as set forth in SEQ ID NO: 11, SEQ ID NO: 38 and SEQ ID NO: 72, respectively.

Preferably, the heavy chain variable region comprises an amino acid sequence as set forth in SEQ ID NO: 149 or SEQ ID NO: 144 and the light chain comprises an amino acid sequence as set forth in SEQ ID NO: 168.

The amino acid sequence of the first protein functional region above is shown in Table e below.

TABLE e
The amino acid sequences related to the
first protein functional region of
the bispecific antibody

| | | Corre- | | Corre- | | Corre- |
|---|---|---|---|---|---|---|
| First | | spond- | | spond- | | spond- |
| protein | SEQ | ing | SEQ | ing | SEQ | ing |
| functional | ID | se- | ID | se- | ID | se- |
| region | NOs: | quences | NOs: | quences | NOs: | quences |
| Heavy chain | 11 | GFTFSTY | 38 | RSKY | 72 | HGNFGN |
| variable | | | | NNYA | | SYVSWF |
| region HCDR | | | | | | AY |
| Light chain | 101 | RSSTGA | 116 | GTNK | 131 | ALWYS |
| variable | | VTTSNY | | RAP | | NLWV |
| region LCDR | | AN | | | | |
| First | | | | | | |
| protein | SEQ | | | | | |
| functional | ID | | | | | |

| region | NOs: | Corresponding sequences |
|---|---|---|
| Heavy chain | 144 | EVQLLESGGGLVQPGGSLRLSCAAS |
| variable | | GFTFSTYAMNWVRQAPGKGLEWVSR |
| region | | IRSKYNNYATYYADSVKDRFTISRD |
| | | DSKSTLYLQMNSLRAEDTAVYYCVR |
| | | HGNFGNSYVSWFAYWGQGTLVTVSS |
| | 149 | EVQLVESGGGLVQPGGSLKLSCAAS |
| | | GFTFSTYAMNWVRQASGKGLEWVGR |
| | | IRSKYNNYATYYADSVKDRFTISRD |
| | | DSKNTAYLQMNSLKTEDTAVYYCTR |
| | | HGNFGNSYVSWFAYWGQGTLVTVSS |
| Light chain | 168 | QAVVTQEPSLTVSPGGTVTLTCRSS |
| variable | | TGAVTTSNYANWVQQKPGQAPRGLI |
| region | | GTNKRAPWTPARFSGSLLGDKAALT |
| | | LLGAQPEDEAEYFCALWYSNLWVG |
| | | FGGGTKLTVL |

Preferably, the bispecific antibody comprises three polypeptide chains in the following forms:
two N-termini of the Fc are linked to the Fab and the VH, respectively; preferably, the bispecific antibody has a first polypeptide chain as shown in formula: VH_{CLDN18.2}-VH_{CLDN18.2}-hinge-CH2-CH3 or VH_{CLDN18.2}-VH_{CLDN18.2}-VH_{CLDN18.2}-hinge-CH2-CH3, a second polypeptide chain as shown in formula: VH_{CD3}-CH1-hinge-CH2-CH3 and a third polypeptide chain as shown in formula: VL_{CD3}-CL;
alternatively, one C-terminus of the HCAb is linked to a VH or VL of the Fab; preferably, the bispecific antibody has a first polypeptide chain as shown in formula: VH_{CLDN18.2}-hinge-CH2-CH3, a second polypeptide chain as shown in formula: VH_{CLDN18.2}-hinge-CH2-CH3-VH_{CD3}-CH1 and a third polypeptide chain as shown in formula: VL_{CD3}-CL; alternatively, the bispecific antibody has a first polypeptide chain as shown in formula: VH_{CLDN18.2}-hinge-CH2-CH3, a second polypeptide chain as shown in formula: VH_{CLDN18.2}-hinge-CH2-CH3-VL_{CD3}-CL and a third polypeptide chain as shown in formula: VH_{CD3}-CH1; alternatively, the bispecific antibody has a first polypeptide chain as shown in formula: VH_{CLDN18.2}-VH_{CLDN18.2}-hinge-CH2-CH3, a second polypeptide chain as shown in formula: VH_{CLDN18.2}-VH_{CLDN18.2}-hinge-CH2-CH3-VH_{CD3}-CH1 and a third polypeptide chain as shown in formula: VL_{CD3}-CL; alternatively, the bispecific antibody has a first polypeptide chain as shown in formula: VH_{CLDN18.2}-VH_{CLDN18.2}-hinge-CH2-CH3, a second polypeptide chain as shown in formula: VH_{CLDN18.2}-VH_{CLDN18.2}-hinge-CH2-CH3-VL_{CD3}-CL and a third polypeptide chain as shown in formula: VH_{CD3}-CH1;
alternatively, the N-terminus of the heavy chain of the Fab is linked to one VH_{CLDN18.2}, the C-terminus of the heavy chain is linked to one N-terminus of the Fc, and the C-terminus of the VHs linked in series is linked to the other N-terminus of the Fc; preferably, the bispecific antibody has a first polypeptide chain as shown in formula: VH_{CLDN18.2}-VH_{CLDN18.2}-hinge-CH2-CH3, a second polypeptide chain as shown in formula: VH_{CLDN18.2}-VH_{CD3}-CH1-hinge-CH2-CH3 and a third polypeptide chain as shown in formula: VL_{CD3}-CL.

More preferably, different functional units such as VH, CH2-CH3 and VL are operably linked by linker peptides preferably comprising an amino acid sequence as set forth in any one of SEQ ID NOs: 244-248, preferably the sequence set forth in SEQ ID NO: 246. See Table f for details.

TABLE f
Linker peptide sequences

| SEQ ID NOs: | Corresponding sequences | Names |
|---|---|---|
| 244 | GGGGS | GS_5 |
| 245 | GGGGSGGGGS | GS_10 |
| 246 | GGGGSGGGGSGGGGS | GS_15 |
| 247 | GGGGSGGGGSGGGGSGGGGS | GS_20 |
| 248 | GGGGSGGGGSGGGGSGGGGSGGGGS | GS_25 |

In one embodiment of the present invention, two N-termini of the Fc are linked to the Fab and the VH, respectively; preferably, the bispecific antibody has a first polypeptide chain as shown in formula: VH_{CLDN18.2}-linker peptide-VH_{CLDN18.2}-hinge-CH2-CH3 or VH_{CLDN18.2}-linker peptide-VH_{CLDN18.2}-linker peptide-VH_{CLDN18.2}-hinge-CH2-CH3, a second polypeptide chain as shown in formula: VH_{CD3}-CH1-hinge-CH2-CH3 and a third polypeptide chain as shown in formula: VL_{CD3}-CL; see structures (1) and (7) of FIG. 4 for specific examples;

alternatively, one C-terminus of the HCAb is linked to a VH or VL of the Fab; preferably, the bispecific antibody has a first polypeptide chain as shown in formula: VH_{CLDN18.2}-hinge-CH2-CH3, a second polypeptide chain as shown in formula: VH_{CLDN18.2}-hinge-CH2-CH3-linker peptide-VH_{CD3}-CH1 and a third polypeptide chain as shown in formula: VL_{CD3}-CL; alternatively, the bispecific antibody has a first polypeptide chain as shown in formula: VH_{CLDN18.2}-hinge-CH2-CH3, a second polypeptide chain as shown in formula: VH_{CLDN18.2}-hinge-CH2-CH3-linker peptide-VL_{CD3}-CL and a third polypeptide chain as shown in formula: VH_{CD3}-CH1; see structures (2) and (3) of FIG. 4 for specific examples;
alternatively, one C-terminus of the VH-HCAb is linked to a VH or VL of the Fab; preferably, the bispecific antibody has a first polypeptide chain as shown in formula: VH_{CLDN18.2}-linker peptide-VH_{CLDN18.2}-hinge-CH2-CH3, a second polypeptide chain as shown in formula: VH_{CLDN18.2}-linker peptide-VH_{CLDN18.2}-hinge-CH2-CH3-linker peptide-VH_{CD3}-CH1 and a third polypeptide chain as shown in formula: VL_{CD3}-CL; alternatively, the bispecific antibody has a first polypeptide chain as shown in formula: VH_{CLDN18.2}-linker peptide-VH_{CLDN18.2}-hinge-CH2-CH3, a second polypeptide chain as shown in formula: VH_{CLDN18.2}-linker peptide-VH_{CLDN18.2}-hinge-CH2-CH3-linker peptide-VL_{CD3}-CL and a third polypeptide chain as shown in formula: VH_{CD3}-CH1; see structures (4) and (5) of FIG. 4 for specific examples;
alternatively, the N-terminus of the heavy chain of the Fab is linked to one VH_{CLDN18.2}, the C-terminus of the heavy chain is linked to one N-terminus of the Fc, and the C-terminus of the VHs linked in series is linked to the other N-terminus of the Fc; preferably, the bispecific antibody has a first polypeptide chain as shown in formula: VH_{CLDN18.2}-linker peptide-VH_{CLDN18.2}-hinge-CH2-CH3, a second polypeptide chain as shown in formula: VH_{CLDN18.2}-linker peptide-VH_{CD3}-CH1-hinge-CH2-CH3 and a third polypeptide chain as shown in formula: VL_{CD3}-CL; see structure (6) of FIG. 4 for a specific example.

In a specific embodiment of the present invention, the first polypeptide chain comprises an amino acid sequence as set forth in SEQ ID NO: 214, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 219, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 220, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 221, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 222, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 223, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 224, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 225, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 226, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 227, the second polypeptide chain comprises an amino acid sequence as set forth in SEQ ID NO: 228, and the third polypeptide chain comprises an amino acid sequence as set forth in SEQ ID NO: 200;
alternatively, the first polypeptide chain comprises an amino acid sequence as set forth in SEQ ID NO: 227, the second polypeptide chain comprises an amino acid sequence as set forth in SEQ ID NO: 230, and the third polypeptide chain comprises an amino acid sequence as set forth in SEQ ID NO: 229;
alternatively, the first polypeptide chain comprises an amino acid sequence as set forth in SEQ ID NO: 219, the second polypeptide chain comprises an amino acid sequence as set forth in SEQ ID NO: 231, and the third polypeptide chain comprises an amino acid sequence as set forth in SEQ ID NO: 200;
alternatively, the first polypeptide chain comprises an amino acid sequence as set forth in SEQ ID NO: 219, the second polypeptide chain comprises an amino acid sequence as set forth in SEQ ID NO: 232, and the third polypeptide chain comprises an amino acid sequence as set forth in SEQ ID NO: 229;
alternatively, the first polypeptide chain comprises an amino acid sequence as set forth in SEQ ID NO: 219, the second polypeptide chain comprises an amino acid sequence as set forth in SEQ ID NO: 233, and the third polypeptide chain comprises an amino acid sequence as set forth in SEQ ID NO: 200;
alternatively, the first polypeptide chain comprises an amino acid sequence as set forth in SEQ ID NO: 234, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 219, the second polypeptide chain comprises an amino acid sequence as set forth in SEQ ID NO: 209, and the third polypeptide chain comprises an amino acid sequence as set forth in SEQ ID NO: 200;
alternatively, the first polypeptide chain comprises an amino acid sequence as set forth in SEQ ID NO: 221, the second polypeptide chain comprises an amino acid sequence as set forth in SEQ ID NO: 209, and the third polypeptide chain comprises an amino acid sequence as set forth in SEQ ID NO: 200;
alternatively, the first polypeptide chain comprises an amino acid sequence as set forth in SEQ ID NO: 236, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 236, the second polypeptide chain comprises an amino acid sequence as set forth in SEQ ID NO: 237, and the third polypeptide chain comprises an amino acid sequence as set forth in SEQ ID NO: 200;
alternatively, the first polypeptide chain comprises an amino acid sequence as set forth in SEQ ID NO: 238, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 239, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 240, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 241, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 242, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 243, the second polypeptide chain comprises an amino acid sequence as set forth in SEQ ID NO: 235, and the third polypeptide chain comprises an amino acid sequence as set forth in SEQ ID NO: 200.

Information about the sequences in the specific embodiment above is shown in Table g below.

TABLE g
Information about the polypeptide chain
sequences of the bispecific antibody

| SEQ | |
|---|---|
| ID | |
| NOs: | Corresponding sequences |
| 200 | QAVVTQEPSLTVSPGGTVTLTCRSSTGAVTT |
| | SNYANWVQQKPGQAPRGLIGGTNKRAPWTP |
| | ARFSGSLLGDKAALTLLGAQPEDEAEYFCAL |
| | WYSNLWVFGGGTKLTVLGQPKAAPSVTLFPP |
| | SSEELQANKATLVCLISDFYPGAVTVAWKAD |
| | SSPVKAGVETTTPSKQSNNKYAASSYLSLTP |
| | E |
| | QWKSHRSYSCQVTHEGSTVEKTVAPTECS |
| 209 | EVQLLESGGGLVQPGGSLRLSCAASGFTFST |
| | YAMNWVRQAPGKGLEWVSRIRSKYNNYATY |
| | YADSVKDRFTISRDDSKSTLYLQMNSLRAED |
| | TAVYYCVRHGNFGNSYVSWFAYWGQGTLV |
| | TVSSASTKGPSVFPLAPSSKSTSGGTAALGC |
| | LVKDYFPEPVTVSWNSGALTSGVHTFPAVLQ |
| | S |
| | SGLYSLSSVVTVPSSSLGTQTYICNVNHKPS |
| | NTKVDKKVEPKSCDKTHTCPPCPAPEAAGGP |
| | S |
| | VFLFPPKPKDTLMISRTPEVTCVVVDVSHED |
| | PEVKFNWYVDGVEVHNAKTKPREEQYNSTYR |
| | VVSVLTVLHQDWLNGKEYKCKVSNKALPAPI |
| | EKTISKAKGQPREPQVYTLPPCREEMTKNQV |
| | SLWCLVKGFYPSDIAVEWESNGQPENNYKTT |
| | PPVLDSDGSFFLYSKLTVDKSRWQQGNVFSC |
| | SVMHEALHNHYTQKSLSLSPGK |
| 213 | EVQLVESGGGLVQPGGSLKLSCAASGFTFST |
| | YAMNWVRQASGKGLEWVGRIRSKYNNYATY |
| | YADSVKDRFTISRDDSKNTAYLQMNSLKTED |
| | TAVYYCTRHGNFGNSYVSWFAYWGQGTLV |
| | TVSSASTKGPSVFPLAPSSKSTSGGTAALGC |
| | LVKDYFPEPVTVSWNSGALTSGVHTFPAVLQ |
| | S |
| | SGLYSLSSVVTVPSSSLGTQTYICNVNHKPS |
| | NTKVDKKVEPKSCDKTHTCPPCPAPEAAGGP |
| | S |
| | VFLFPPKPKDTLMISRTPEVTCVVVDVSHED |
| | PEVKFNWYVDGVEVHNAKTKPREEQYNSTYR |
| | VVSVLTVLHQDWLNGKEYKCKVSNKALPAPI |
| | EKTISKAKGQPREPQVYTLPPCREEMTKNQV |
| | SLWCLVKGFYPSDIAVEWESNGQPENNYKTT |
| | PPVLDSDGSFFLYSKLTVDKSRWQQGNVFSC |
| | SVMHEALHNHYTQKSLSLSPGK |
| 214 | EVQLVESGGGLVQPGGSLRLSCAASGFTFSS |
| | YWMHWVRQAPGKGLEWVSYINNDGSSTRYA |
| | DSVKGRFTISRDNAKNTLYLQMNSLRAEDTA |
| | VYYCTEAPPYGNYERDYWGQGTLVTVSSGG |
| | GGSGGGGSGGGGSEVQLVESGGGLVQPGGSL |
| | RLSCAASGFTFSSYWMHWVRQAPGKGLEW |
| | VSYINNDGSSTRYADSVKGRFTISRDNAKNT |
| | LYLQMNSLRAEDTAVYYCTEAPPYGNYERDY |
| | WGQGTLVTVSSASEPKSSDKTHTCPPCPAPE |
| | AAGGPSVFLFPPKPKDTLMISRTPEVTCVVV |
| | D |
| | VSHEDPEVKFNWYVDGVEVHNAKTKPREEQY |
| | NSTYRVVSVLTVLHQDWLNGKEYKCKVSN |
| | KALPAPIEKTISKAKGQPREPQVCTLPPSRE |
| | EMTKNQVSLSCAVKGFYPSDIAVEWESNGQP |
| | E |
| | NNYKTTPPVLDSDGSFFLVSKLTVDKSRWQQ |
| | GNVFSCSVMHEALHNHYTQKSLSLSPGK |
| 219 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRINSDGSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGGPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 220 | EVQLVESGGGLVQPGGSLRLSCAASGFTFSS |
| | YWMYWVRQVPGKGLVWVSHINSDGSTTQYA |
| | DSVKGRFTISRDNAKNMLYLQMNSLRAEDTA |
| | VYYCARDFPVLGGSHFDFQHWGQGTLVTVS |
| | SGGGGSGGGGSGGGGSEVQLVESGGGLVQPG |
| | GSLRLSCAASGFTFSSYWMYWVRQVPGKG |
| | LVWVSHINSDGSTTQYADSVKGRFTISRDNA |
| | KNMLYLQMNSLRAEDTAVYYCARDFPVLGG |
| | SHFDFQHWGQGTLVTVSSASEPKSSDKTHTC |
| | PPCPAPEAAGGPSVFLFPPKPKDTLMISRTP |
| | EV |
| | TCVVVDVSHEDPEVKFNWYVDGVEVHNAKTK |
| | PREEQYNSTYRVVSVLTVLHQDWLNGKEY |
| | KCKVSNKALPAPIEKTISKAKGQPREPQVCT |
| | LPPSREEMTKNQVSLSCAVKGFYPSDIAVEW |
| | E |
| | SNGQPENNYKTTPPVLDSDGSFFLVSKLTVD |
| | KSRWQQGNVFSCSVMHEALHNHYTQKSLSLS |
| | PGK |
| 221 | EVQLVESGGGLVQPGGSLRLSCAASGFTFSS |
| | YWMHWVRQVPGKGLVWVSRISSTGSRTIYAD |
| | SVKGRFTISRDNAKNTLYLQMNSLRAEDTAV |
| | YYCARGEDHDILTGYPIRGQGTTVTVSSGGG |
| | GSGGGGSGGGGSEVQLVESGGGLVQPGGSLR |
| | LSCAASGFTFSSYWMHWVRQVPGKGLVWV |
| | SRISSTGSRTIYADSVKGRFTISRDNAKNTL |
| | YLQMNSLRAEDTAVYYCARGEDHDILTGYPI |
| | RG |
| | QGTTVTVSSASEPKSSDKTHTCPPCPAPEAA |
| | GGPSVFLFPPKPKDTLMISRTPEVTCVVVDV |
| | SH |
| | EDPEVKFNWYVDGVEVHNAKTKPREEQYNST |
| | YRVVSVLTVLHQDWLNGKEYKCKVSNKAL |
| | PAPIEKTISKAKGQPREPQVCTLPPSREEMT |
| | KNQVSLSCAVKGFYPSDIAVEWESNGQPENN |
| | Y |
| | KTTPPVLDSDGSFFLVSKLTVDKSRWQQGNV |
| | FSCSVMHEALHNHYTQKSLSLSPGK |
| 222 | EVQLVESGGGLVQPGGSLRLSCAASGFTFSS |
| | YWMHWVRQVPGKGLVWVSRINFDSSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRINFDSSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGGPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 223 | EVQLVESGGGLVQPGGSLRLSCAASGFTFSS |
| | YWMHWVRQVPGKGLVWVSRISSAGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRISSAGSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGGPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 224 | EVQLVESGGGLVQPGGSLRLSCAASGFTFSS |
| | YWMHWVRQVPGKGLVWVSRIDSRGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRIDSRGSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGGPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 225 | EVQLVESGGGLVQPGGSLRLSCAASGFTFSS |
| | YWMHWVRQVPGKGLVWVSRISSDASRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRISSDASRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGGPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 226 | EVQLVESGGGLVQPGGSLRLSCAASGFTFSS |
| | YWMYWVRQVPGKGLVWVSHINPLGSTTQYA |
| | DSVKGRFTISRDNAKNMLYLQMNSLRAEDTA |
| | VYYCARDFPVLGGSHFDFQHWGQGTLVTVS |
| | SGGGGSGGGGSGGGGSEVQLVESGGGLVQPG |
| | GSLRLSCAASGFTFSSYWMYWVRQVPGKG |
| | LVWVSHINPLGSTTQYADSVKGRFTISRDNA |
| | KNMLYLQMNSLRAEDTAVYYCARDFPVLGG |
| | SHFDFQHWGQGTLVTVSSASEPKSSDKTHTC |
| | PPCPAPEAAGGPSVFLFPPKPKDTLMISRTP |
| | EV |
| | TCVVVDVSHEDPEVKFNWYVDGVEVHNAKTK |
| | PREEQYNSTYRVVSVLTVLHQDWLNGKEY |
| | KCKVSNKALPAPIEKTISKAKGQPREPQVCT |
| | LPPSREEMTKNQVSLSCAVKGFYPSDIAVEW |
| | E |
| | SNGQPENNYKTTPPVLDSDGSFFLVSKLTVD |
| | KSRWQQGNVFSCSVMHEALHNHYTQKSLSLS |
| | PGK |
| 227 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSAS |
| | EPKSSDKTHTCPPCPAPEAAGGPSVFLFPPK |
| | PKDTLMISRTPEVTCVVVDVSHEDPEVKFNW |
| | Y |
| | VDGVEVHNAKTKPREEQYNSTYRVVSVLTVL |
| | HQDWLNGKEYKCKVSNKALPAPIEKTISKA |
| | KGQPREPQVCTLPPSREEMTKNQVSLSCAVK |
| | GFYPSDIAVEWESNGQPENNYKTTPPVLDSD |
| | GSFFLVSKLTVDKSRWQQGNVFSCSVMHEAL |
| | HNHYTQKSLSLSPGK |
| 228 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSAS |
| | EPKSSDKTHTCPPCPAPEAAGGPSVFLFPPK |
| | PKDTLMISRTPEVTCVVVDVSHEDPEVKFNW |
| | Y |
| | VDGVEVHNAKTKPREEQYNSTYRVVSVLTVL |
| | HQDWLNGKEYKCKVSNKALPAPIEKTISKA |
| | KGQPREPQVYTLPPCREEMTKNQVSLWCLVK |
| | GFYPSDIAVEWESNGQPENNYKTTPPVLDSD |
| | GSFFLYSKLTVDKSRWQQGNVFSCSVMHEAL |
| | HNHYTQKSLSLSPGGGGGSGGGGSGGGGSR |
| | TEVQLLESGGGLVQPGGSLRLSCAASGFTFS |
| | TYAMNWVRQAPGKGLEWVSRIRSKYNNYAT |
| | YYADSVKDRFTISRDDSKSTLYLQMNSLRAE |
| | DTAVYYCVRHGNFGNSYVSWFAYWGQGTL |
| | VTVSSASTKGPSVFPLAPSSKSTSGGTAALG |
| | CLVKDYFPEPVTVSWNSGALTSGVHTFPAVL |
| | Q |
| | SSGLYSLSSVVTVPSSSLGTQTYICNVNHKP |
| | SNTKVDKKVEPKSC |
| 229 | EVQLLESGGGLVQPGGSLRLSCAASGFTFST |
| | YAMNWVRQAPGKGLEWVSRIRSKYNNYATY |
| | YADSVKDRFTISRDDSKSTLYLQMNSLRAED |
| | TAVYYCVRHGNFGNSYVSWFAYWGQGTLV |
| | TVSSASTKGPSVFPLAPSSKSTSGGTAALGC |
| | LVKDYFPEPVTVSWNSGALTSGVHTFPAVLQ |
| | S |
| | SGLYSLSSVVTVPSSSLGTQTYICNVNHKPS |
| | NTKVDKKVEPKSC |
| 230 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSAS |
| | EPKSSDKTHTCPPCPAPEAAGGPSVFLFPPK |
| | PKDTLMISRTPEVTCVVVDVSHEDPEVKFNW |
| | Y |
| | VDGVEVHNAKTKPREEQYNSTYRVVSVLTVL |
| | HQDWLNGKEYKCKVSNKALPAPIEKTISKA |
| | KGQPREPQVYTLPPCREEMTKNQVSLWCLVK |
| | GFYPSDIAVEWESNGQPENNYKTTPPVLDSD |
| | GSFFLYSKLTVDKSRWQQGNVFSCSVMHEAL |
| | HNHYTQKSLSLSPGGGGGSGGGGSGGGGSR |
| | TQAVVTQEPSLTVSPGGTVTLTCRSSTGAVT |
| | TSNYANWVQQKPGQAPRGLIGGTNKRAPWTP |
| | ARFSGSLLGDKAALTLLGAQPEDEAEYFCAL |
| | WYSNLWVFGGGTKLTVLGQPKAAPSVTLFPP |
| | SSEELQANKATLVCLISDFYPGAVTVAWKAD |
| | SSPVKAGVETTTPSKQSNNKYAASSYLSLTP |
| | EQWKSHRSYSCQVTHEGSTVEKTVAPTECS |
| 231 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRINSDGSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGGPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVYTLPPCREE |
| | MTKNQVSLWCLVKGFYPSDIAVEWESNGQPE |
| | N |
| | NYKTTPPVLDSDGSFFLYSKLTVDKSRWQQG |
| | NVFSCSVMHEALHNHYTQKSLSLSPGGGGGS |
| | GGGGSGGGGSRTEVQLLESGGGLVQPGGSLR |
| | LSCAASGFTFSTYAMNWVRQAPGKGLEWVS |
| | RIRSKYNNYATYYADSVKDRFTISRDDSKST |
| | LYLQMNSLRAEDTAVYYCVRHGNFGNSYVS |
| | WFAYWGQGTLVTVSSASTKGPSVFPLAPSSK |
| | STSGGTAALGCLVKDYFPEPVTVSWNSGALT |
| | SGVHTFPAVLQSSGLYSLSSVVTVPSSSLGT |
| | QTYICNVNHKPSNTKVDKKVEPKSC |
| 232 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRINSDGSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGGPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVYTLPPCREE |
| | MTKNQVSLWCLVKGFYPSDIAVEWESNGQPE |
| | N |
| | NYKTTPPVLDSDGSFFLYSKLTVDKSRWQQG |
| | NVFSCSVMHEALHNHYTQKSLSLSPGGGGGS |
| | GGGGSGGGGSRTQAVVTQEPSLTVSPGGTVT |
| | LTCRSSTGAVTTSNYANWVQQKPGQAPRGLI |
| | GGTNKRAPWTPARFSGSLLGDKAALTLLGAQ |
| | PEDEAEYFCALWYSNLWVFGGGTKLTVLGQ |
| | PKAAPSVTLFPPSSEELQANKATLVCLISDF |
| | YPGAVTVAWKADSSPVKAGVETTTPSKQSNN |
| | K |
| | YAASSYLSLTPEQWKSHRSYSCQVTHEGSTV |
| | EKTVAPTECS |
| 233 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLLESGGGLVQPGGSL |
| | RLSCAASGFTFSTYAMNWVRQAPGKGLEW |
| | VSRIRSKYNNYATYYADSVKDRFTISRDDSK |
| | STLYLQMNSLRAEDTAVYYCVRHGNFGNSY |
| | VSWFAYWGQGTLVTVSSASTKGPSVFPLAPS |
| | SKSTSGGTAALGCLVKDYFPEPVTVSWNSGA |
| | LTSGVHTFPAVLQSSGLYSLSSVVTVPSSSL |
| | GTQTYICNVNHKPSNTKVDKKVEPKSCDKTH |
| | T |
| | CPPCPAPEAAGGPSVFLFPPKPKDTLMISRT |
| | PEVTCVVVDVSHEDPEVKFNWYVDGVEVHNA |
| | KTKPREEQYNSTYRVVSVLTVLHQDWLNGKE |
| | YKCKVSNKALPAPIEKTISKAKGQPREPQVY |
| | TLPPCREEMTKNQVSLWCLVKGFYPSDIAVE |
| | WESNGQPENNYKTTPPVLDSDGSFFLYSKLT |
| | VDKSRWQQGNVFSCSVMHEALHNHYTQKSLS |
| | LSPGK |
| 234 | EVQLVESGGGLVQPGGSLRLSCAASGFTFSS |
| | YWMHWVRQVPGKGLVWVSRINSDGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRINSDGSRTIYADSVKGRFTISRDNAKNT |
| | LYLQMNSLRAEDTAVYYCARGEDHDILTGYP |
| | I |
| | RGQGTTVTVSSGGGGSGGGGSGGGGSEVQLV |
| | ESGGGLVQPGGSLRLSCAASGFTFSSYWMH |
| | WVRQVPGKGLVWVSRINSDGSRTIYADSVKG |
| | RFTISRDNAKNTLYLQMNSLRAEDTAVYYC |
| | ARGEDHDILTGYPIRGQGTTVTVSSASEPKS |
| | SDKTHTCPPCPAPEAAGGPSVFLFPPKPKDT |
| | LM |
| | ISRTPEVTCVVVDVSHEDPEVKFNWYVDGVE |
| | VHNAKTKPREEQYNSTYRVVSVLTVLHQDW |
| | LNGKEYKCKVSNKALPAPIEKTISKAKGQPR |
| | EPQVCTLPPSREEMTKNQVSLSCAVKGFYPS |
| | D |
| | IAVEWESNGQPENNYKTTPPVLDSDGSFFLV |
| | SKLTVDKSRWQQGNVFSCSVMHEALHNHYT |
| | QKSLSLSPGK |
| 235 | EVQLVESGGGLVQPGGSLKLSCAASGFTFST |
| | YAMNWVRQASGKGLEWVGRIRSKYNNYATY |
| | YADSVKDRFTISRDDSKNTAYLQMNSLKTED |
| | TAVYYCTRHGNFGNSYVSWFAYWGQGTLV |
| | TVSSASTKGPSVFPLAPSSKSTSGGTAALGC |
| | LVKDYFPEPVTVSWNSGALTSGVHTFPAVLQ |
| | S |
| | SGLYSLSSVVTVPSSSLGTQTYICNVNHKPS |
| | NTKVDKKVEPKSCDKTHTCPPCPAPEAAGAP |
| | S |
| | VFLFPPKPKDTLMISRTPEVTCVVVDVSHED |
| | PEVKFNWYVDGVEVHNAKTKPREEQYNSTYR |
| | VVSVLTVLHQDWLNGKEYKCKVSNKALPAPI |
| | EKTISKAKGQPREPQVYTLPPCREEMTKNQV |
| | SLWCLVKGFYPSDIAVEWESNGQPENNYKTT |
| | PPVLDSDGSFFLYSKLTVDKSRWQQGNVFSC |
| | SVMHEALHNHYTQKSLSLSPGK |
| 236 | EVQLVESGGGLVQPGGSLRLSCAASGFTFSS |
| | YWMHWVRQVPGKGLVWVSRISSDASRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRISSDASRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGAPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 237 | EVQLLESGGGLVQPGGSLRLSCAASGFTFST |
| | YAMNWVRQAPGKGLEWVSRIRSKYNNYATY |
| | YADSVKDRFTISRDDSKSTLYLQMNSLRAED |
| | TAVYYCVRHGNFGNSYVSWFAYWGQGTLV |
| | TVSSASTKGPSVFPLAPSSKSTSGGTAALGC |
| | LVKDYFPEPVTVSWNSGALTSGVHTFPAVLQ |
| | S |
| | SGLYSLSSVVTVPSSSLGTQTYICNVNHKPS |
| | NTKVDKKVEPKSCDKTHTCPPCPAPEAAGAP |
| | S |
| | VFLFPPKPKDTLMISRTPEVTCVVVDVSHED |
| | PEVKFNWYVDGVEVHNAKTKPREEQYNSTYR |
| | VVSVLTVLHQDWLNGKEYKCKVSNKALPAPI |
| | EKTISKAKGQPREPQVYTLPPCREEMTKNQV |
| | SLWCLVKGFYPSDIAVEWESNGQPENNYKTT |
| | PPVLDSDGSFFLYSKLTVDKSRWQQGNVFSC |
| | SVMHEALHNHYTQKSLSLSPGK |
| 238 | EVQLVESGGGLVQPGGSLRLSCAASGFTFSS |
| | YWMHWVRQVPGKGLVWVSRISSTGSRTIYAD |
| | SVKGRFTISRDNAKNTLYLQMNSLRAEDTAV |
| | YYCARGEDHDILTGYPIRGQGTTVTVSSGGG |
| | GSGGGGSGGGGSEVQLVESGGGLVQPGGSLR |
| | LSCAASGFTFSSYWMHWVRQVPGKGLVWV |
| | SRISSTGSRTIYADSVKGRFTISRDNAKNTL |
| | YLQMNSLRAEDTAVYYCARGEDHDILTGYPI |
| | RG |
| | QGTTVTVSSASEPKSSDKTHTCPPCPAPEAA |
| | GAPSVFLFPPKPKDTLMISRTPEVTCVVVDV |
| | SH |
| | EDPEVKFNWYVDGVEVHNAKTKPREEQYNST |
| | YRVVSVLTVLHQDWLNGKEYKCKVSNKAL |
| | PAPIEKTISKAKGQPREPQVCTLPPSREEMT |
| | KNQVSLSCAVKGFYPSDIAVEWESNGQPENN |
| | Y |
| | KTTPPVLDSDGSFFLVSKLTVDKSRWQQGNV |
| | FSCSVMHEALHNHYTQKSLSLSPGK |
| 239 | EVQLVESGGGLVQPGGSLRLSCAASGFTFSS |
| | YWMHWVRQVPGKGLVWVSRISSAGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRISSAGSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGAPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 240 | EVQLVESGGGLVQPGGSLRLSCAASGFTFSS |
| | YWMHWVRQVPGKGLVWVSRIDSRGSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRIDSRGSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGAPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 241 | EVQLVESGGGLVQPGGSLRLSCAASGFTFSS |
| | YWMHWVRQVPGKGLVWVSRINFDSSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRINFDSSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGAPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 242 | EVQLVESGGGLVQPGGSLRLSCAASGFTFSS |
| | YWMHWVRQVPGKGLVWVSRINHDSSRTIYA |
| | DSVKGRFTISRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VSRINHDSSRTIYADSVKGRFTISRDNAKNT |
| | LYLQMNSLRAEDTAVYYCARGEDHDILTGYP |
| | I |
| | RGQGTTVTVSSASEPKSSDKTHTCPPCPAPE |
| | AAGAPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |
| 243 | EVQLVESGGGLVQPGGSLRLSCAASGFTFSS |
| | YWMHWVRQVPGKGLVWVCRISSDASRTIYA |
| | DSVKGRFTCSRDNAKNTLYLQMNSLRAEDTA |
| | VYYCARGEDHDILTGYPIRGQGTTVTVSSGG |
| | GGSGGGGSGGGGSEVQLVESGGGLVQPGGSL |
| | RLSCAASGFTFSSYWMHWVRQVPGKGLVW |
| | VCRISSDASRTIYADSVKGRFTCSRDNAKNT |
| | LYLQMNSLRAEDTAVYYCARGEDHDILTGYP |
| | I |
| | RGQGTTVTVSSASEPKSSDKTHTCPPCPAPE |
| | AAGAPSVFLFPPKPKDTLMISRTPEVTCVVV |
| | DV |
| | SHEDPEVKFNWYVDGVEVHNAKTKPREEQYN |
| | STYRVVSVLTVLHQDWLNGKEYKCKVSNK |
| | ALPAPIEKTISKAKGQPREPQVCTLPPSREE |
| | MTKNQVSLSCAVKGFYPSDIAVEWESNGQPE |
| | N |
| | NYKTTPPVLDSDGSFFLVSKLTVDKSRWQQG |
| | NVFSCSVMHEALHNHYTQKSLSLSPGK |

To solve the above technical problems, a third aspect of the present invention provides an isolated nucleic acid encoding the antibody according to the first aspect of the present invention or the bispecific antibody according to the second aspect of the present invention.

The preparation method for the nucleic acid is a conventional preparation method in the art, and preferably comprises the following steps: obtaining a nucleic acid molecule encoding the above antibody by gene cloning technology, or obtaining a nucleic acid molecule encoding the above antibody by artificial complete sequence synthesis.

It is known to those skilled in the art that substitutions, deletions, alterations, insertions or additions may be appropriately introduced into the base sequence encoding the amino acid sequence of the above antibody to provide a polynucleotide homologue. The polynucleotide homologue of the present invention may be produced by substituting, deleting or adding one or more bases of a gene encoding the antibody sequence within a range in which the activity of the antibody is maintained.

To solve the above technical problems, a fourth aspect of the present invention provides a recombinant expression vector comprising the isolated nucleic acid according to the third aspect of the present invention. The recombinant expression vector may be obtained by using conventional methods in the art, i.e., by linking the nucleic acid molecule of the present invention to various expression vectors. The expression vector is any conventional vector in the art, provided that it can carry the aforementioned nucleic acid molecule.

Preferably, the recombinant expression vector is a plasmid, a cosmid, a phage or a viral vector, wherein the viral vector is preferably a retroviral vector, a lentiviral vector, an adenoviral vector or an adeno-associated viral vector.

To solve the above technical problems, a fifth aspect of the present invention provides a transformant comprising the recombinant expression vector according to the fourth aspect of the present invention in a host cell; preferably, the host cell is an E. coli TG1, BL21 cell, or CHO-K1 cell.

The recombinant expression transformant may be prepared by using conventional methods in the art, e.g., by transforming the above recombinant expression vector into a host cell. The host cell is any conventional host cell in the art, provided that it can enable the stable replication of the above recombinant expression vector and the nucleic acid carried can be efficiently expressed. Preferably, the host cell is an E. coli TG1 or BL21 cell (expressing a single-chain antibody or an Fab antibody), or a CHO-K1 cell (expressing a full-length IgG antibody). The preferred recombinant expression transformant of the present invention can be obtained by transforming the aforementioned recombinant expression plasmid into a host cell. The transformation method is a conventional transformation method in the art, preferably a chemical transformation method, a heat shock method or an electric transformation method.

In the present invention, the CLDN18.2-targeting antibody may be used to prepare a chimeric antigen receptor (CAR) or the like so as to modify it onto cells such as T cells or NK cells. Thus, a sixth aspect of the present invention provides a chimeric antigen receptor (CAR) comprising the antibody according to the first aspect of the present invention or the bispecific antibody according to the second aspect of the present invention.

For example, the chimeric antigen receptor may comprise the following structure: (a) an extracellular binding domain scFv that specifically recognizes CLDN18.2; (b) a hinge domain; (c) a transmembrane domain; (d) a co-stimulatory intracellular domain; and (e) a signaling domain; wherein the extracellular binding domain comprises the CLDN18.2-targeting antibody according to the first aspect of the present invention.

To solve the above technical problems, a seventh aspect of the present invention provides a genetically modified cell comprising the antibody according to the first aspect of the present invention or the bispecific antibody according to the second aspect of the present invention. Preferably, the genetically modified cell is a eukaryotic cell, preferably an isolated human cell, and more preferably an immune cell such as a T cell or an NK cell.

To solve the above technical problems, an eighth aspect of the present invention provides a method for preparing a bispecific antibody comprising culturing the transformant according to the fifth aspect of the present invention, and obtaining the antibody or the bispecific antibody from the culture.

To solve the above technical problems, a ninth aspect of the present invention provides an antibody-drug conjugate (ADC) comprising a cytotoxic agent, and the antibody according to the first aspect of the present invention or the bispecific antibody according to the second aspect of the present invention; preferably, the cytotoxic agent is MMAF or MMAE.

The preparation method for the antibody-drug conjugate may be a conventional method in the art, and preferably the preparation method described in Doronina, 2006, Bioconjugate Chem. 17, 114-124. Preferably, the preparation method produces antibody-drug conjugates with a minimal low conjugate fraction (LCF) of less than 10%.

The antibody-drug conjugate can be present in any physical form known in the art, preferably as a clear solution.

To solve the above technical problems, a tenth aspect of the present invention provides a pharmaceutical composition comprising the antibody according to the first aspect of the present invention or the bispecific antibody according to the second aspect of the present invention, and a pharmaceutically acceptable carrier.

Preferably, the pharmaceutical composition further comprises one or more of the group consisting of a hormonal agent, a small molecule-targeted agent, a proteasome inhibitor, an imaging agent, a diagnostic agent, a chemotherapeutic agent, an oncolytic drug, a cytotoxic agent, a cytokine, an activator of a costimulatory molecule, an inhibitor of an inhibitory molecule, and a vaccine.

To solve the above technical problems, an eleventh aspect of the present invention provides use of the antibody according to the first aspect of the present invention or the bispecific antibody according to the second aspect of the present invention or the pharmaceutical composition according to the tenth aspect of the present invention in the manufacture of a medicament for the prevention or treatment of a CD3 and/or CLDN18.2-associated disease;
the disease is preferably cancer, wherein the cancer is preferably breast cancer, ovarian cancer, endometrial cancer, renal cancer, melanoma, lung cancer, gastric cancer, liver cancer, esophageal cancer, cervical cancer, head and neck tumor, cholangiocarcinoma, gallbladder cancer, bladder cancer, sarcoma or colorectal cancer; preferably, the cancer is breast cancer, ovarian cancer, endometrial cancer, renal cancer or cholangiocarcinoma; more preferably, the cancer is breast cancer.

To solve the above technical problems, a twelfth aspect of the present invention provides a kit comprising the antibody according to the first aspect of the present invention or the bispecific antibody according to the second aspect of the present invention, the chimeric antigen receptor according to the sixth aspect of the present invention, the genetically modified cell according to the seventh aspect of the present invention, or the antibody-drug conjugate according to the ninth aspect of the present invention or the pharmaceutical composition according to the tenth aspect of the present invention;
preferably, the kit further comprises (i) a device for administering the antibody or an antigen-binding fragment thereof or the antibody-drug conjugate or the pharmaceutical composition; and/or (ii) instructions.

To solve the above technical problems, a thirteenth aspect of the present invention provides a kit of parts comprising a kit A and a kit B, wherein:
the kit A comprises the antibody according to the first aspect of the present invention or the bispecific antibody according to the second aspect of the present invention, the chimeric antigen receptor according to the sixth aspect of the present invention, the genetically modified cell according to the seventh aspect of the present invention, the antibody-drug conjugate according to the ninth aspect of the present invention and/or the pharmaceutical composition according to the tenth aspect of the present invention;
the kit B comprises other anti-tumor antibodies or a pharmaceutical composition comprising the other anti-tumor antibodies, and/or one or more of the group consisting of a hormonal agent, a small molecule-targeted agent, a proteasome inhibitor, an imaging agent, a diagnostic agent, a chemotherapeutic agent, an oncolytic drug, a cytotoxic agent, a cytokine, an activator of a co-stimulatory molecule, an inhibitor of an inhibitory molecule, and a vaccine.

The kit A and the kit B may be used simultaneously, or the kit A may be used prior to the use of the kit B, or the kit B may be used prior to the use of the kit A. The sequence of use can be determined according to actual requirements in a specific application.

To solve the above technical problems, a fourteenth aspect of the present invention provides a method for diagnosing, treating and/or preventing a CLDN18.2-mediated disease or disorder comprising administering to a patient in need thereof a therapeutically effective amount of the antibody according to the first aspect of the present invention or the bispecific antibody according to the second aspect of the present invention, the chimeric antigen receptor according to the sixth aspect of the present invention, the antibody-drug conjugate according to the ninth aspect of the present invention or the pharmaceutical composition according to the tenth aspect of the present invention, or treating a patient in need thereof using the kit of parts according to the thirteenth aspect of the present invention.

Preferably, the disease or disorder is a tumor, preferably a CLDN18.2 positive tumor, and more preferably gastric cancer, esophageal cancer, lung cancer, ovarian cancer, melanoma, renal cancer, breast cancer, colorectal cancer, liver cancer, pancreatic cancer, bladder cancer, head and neck cancer, bronchial carcinoma, glioma and/or leukemia.

The “CLDN18.2 positive” used in the present invention refers to the overexpression of the CLDN18.2 protein, e.g., the CLDN18.2-positive cell NUGC4_D8 cell lines; otherwise, it is referred to as “CLDN18.2 negative”.

To solve the above technical problems, a fifteenth aspect of the present invention provides a method of immunodetection or determination of CLDN18.2 comprising using the antibody according to the first aspect of the present invention or the bispecific antibody according to the second aspect of the present invention, the chimeric antigen receptor according to the sixth aspect of the present invention, the antibody-drug conjugate according to the ninth aspect of the present invention, or the pharmaceutical composition according to the tenth aspect of the present invention. Preferably, the detection is for non-diagnostic and/or therapeutic purposes.

To solve the above technical problems, a sixteenth aspect of the present invention provides a combination therapy comprising administering to a patient in need thereof the antibody according to the first aspect of the present invention or the bispecific antibody according the second aspect of the present invention, the chimeric antigen receptor according to the sixth aspect of the present invention, the antibody-drug conjugate according to the ninth aspect of the present invention or the pharmaceutical composition according to the tenth aspect of the present invention, and a second therapeutic agent; the second therapeutic agent preferably comprises other anti-tumor antibodies or a pharmaceutical composition comprising the other anti-tumor antibodies, and/or one or more of the group consisting of a hormonal agent, a small molecule-targeted agent, a proteasome inhibitor, an imaging agent, a diagnostic agent, a chemotherapeutic agent, an oncolytic drug, a cytotoxic agent, a cytokine, an activator of a co-stimulatory molecule, an inhibitor of an inhibitory molecule, and a vaccine.

In the present application, the amino acid sequences of the listed CDRs are all shown according to the Chothia scheme (the sequences in the claims of the present invention are also shown according to the Chothia scheme). However, it is well known to those skilled in the art that the CDRs of an antibody can be defined in the art using a variety of methods, such as the Kabat scheme based on sequence variability (see Kabat et al., Sequences of Proteins of Immunological Interest, Fifth Edition, National Institutes of Health (U.S.), Bethesda, Maryland (1991)), and the Chothia scheme based on the location of the structural loop regions (see J Mol Biol 273:927-48, 1997). In the present application, the Combined scheme comprising the Kabat scheme and the Chothia scheme can also be used to determine the amino acid residues in a variable domain sequence. The Combined scheme combines the Kabat scheme with the Chothia scheme to obtain a larger range. See Table 1 for details. It will be understood by those skilled in the art that unless otherwise specified, the terms “CDR” and “complementary determining region” of a given antibody or a region (e.g., variable region) thereof are construed as encompassing complementary determining regions as defined by any one of the above known schemes described herein. Although the scope claimed in the claims of the present invention is the sequences shown based on the Chothia scheme, the amino acid sequences corresponding to the other CDR-defined schemes shall also fall within the scope of the present invention.

TABLE 1
The definition scheme for the CDRs of the antibody of
the present invention (see http://bioinf.org.uk/abs/)

| | Kabat | Chothia | Combined |
|---|---|---|---|
| VL CDR1 | L24--L34 | L24--L34 | L24-L34 |
| VL CDR2 | L50--L56 | L50--L56 | L50-L56 |
| VL CDR3 | L89--L97 | L89--L97 | L89-L97 |
| VH CDR1 | H31--H35 | H26--H32 | H26-H35 |
| VH CDR2 | H50--H65 | H52--H56 | H50-H65 |
| VH CDR3 | H95--H102 | H95--H102 | H95-H102 |

In the Chothia scheme, Laa-Lbb can refer to an amino acid sequence from position aa to position bb beginning at the N-terminus of the light chain of the antibody; and Haa-Hbb can refer to an amino acid sequence from position aa to position bb beginning at the N-terminus of the heavy chain of the antibody. For example, L24-L34 can refer to the amino acid sequence from position 24 to position 34 according to the Chothia scheme beginning at the N-terminus of the light chain of the antibody; H26-H32 can refer to the amino acid sequence from position 26 to position 32 according to the Chothia scheme beginning at the N-terminus of the heavy chain of the antibody. It is well known to those skilled in the art that there are positions where insertion sites are present in numbering CDRs with the Chothia scheme.

In the present invention, unless otherwise defined, the scientific and technical terms used herein have the meanings generally understood by those skilled in the art. In addition, the laboratory operations of cell culture, molecular genetics, nucleic acid chemistry and immunology used herein are the routine procedures widely used in the corresponding fields. Meanwhile, in order to better understand the present invention, the definitions and explanations of the relevant terms are provided below.

The three-letter codes and single-letter codes for amino acids used in the present invention are known to those skilled in the art, or are described in J. Biol. Chem, 243, p 3558 (1968).

As used herein, the term “include/includes/including” or “comprise/comprises/comprising” is intended to mean that a composition and a method include the elements described but does not exclude other elements; but the case of “consist/consists/consisting of” is also included as the context dictates.

The term “CLDN18.2” includes isotypes, mammalian (e.g., human) CLDN18.2, species homologues of human CLDN18.2 and analogues comprising at least one common epitope with CLDN18.2. The amino acid sequence of CLDN18.2 (e.g., human CLDN18.2) is known in the art, as shown in the NCBI database.

The term “CLDN18.1” includes isotypes, mammalian (e.g., human) CLDN18.1, species homologues of human CLDN18.1 and analogues comprising at least one common epitope with CLDN18.1. The amino acid sequence of CLDN18.1 (e.g., human CLDN18.1) is known in the art, as shown in the NCBI database.

The term “epitope” refers to moieties of an antigen (e.g., human CLDN18.2) that specifically interact with an antibody molecule. The term “competitive” in the present invention refers to the ability of an antibody molecule to interfere with the binding of an anti-CLDN18.2 antibody molecule to a target (e.g., human CLDN18.2). The interference with the binding may be direct or indirect (e.g., through the allosteric modulation of an antibody molecule or target). Competitive binding assays (e.g., FACS assays, ELISA, or BIACORE assays) can be used to determine the extent to which whether an antibody molecule is able to interfere with the binding of another antibody molecule to its target.

The term “antibody” used in the present invention includes an immunoglobulin, which is a tetrapeptide chain structure formed by connection between two identical heavy chains and two identical light chains by interchain disulfide bonds. Immunoglobulins differ in amino acid composition and arrangement of their heavy chain constant regions and therefore in their antigenicity. Accordingly, immunoglobulins can be classified into five classes, or isotypes of immunoglobulins, namely IgM, IgD, IgG, IgA and IgE, with their corresponding heavy chains being the μ, δ, γ, α and ε chains, respectively. The Ig of the same class can be divided into different subclasses according to the differences in amino acid composition of the hinge regions and the number and location of disulfide bonds in the heavy chains; for example, IgG can be divided into IgG1, IgG2, IgG3, and IgG4. Light chains are classified into κ or λ chains by the difference in the constant regions. Each of the five classes of Ig can have a κ chain or a λ chain.

In the present invention, the light chain variable region of the antibody of the present invention may further comprise a light chain constant region comprising a human κ or λ chain or a variant thereof. In the present invention, the heavy chain variable region of the antibody of the present invention may further comprise a heavy chain constant region comprising human IgG1, IgG2, IgG3, IgG4 or a variant thereof.

The sequences of about 110 amino acids of the heavy and light chains of the antibody near the N-terminus vary considerably and thus are referred to as variable regions (V regions); the remaining amino acid sequences near the C-terminus are relatively stable and thus are referred to as constant regions (C regions). The variable regions comprise 3 hypervariable regions (HVRs) and 4 framework regions (FWRs) with relatively conservative sequences. The 3 hypervariable regions determine the specificity of the antibody and thus are also known as complementarity determining regions (CDRs). Each light chain variable region (VL) or heavy chain variable region (VH) consists of 3 CDR regions and 4 FWR regions arranged from the amino-terminus to the carboxy-terminus in the following order: FWR1, CDR1, FWR2, CDR2, FWR3, CDR3, and FWR4. The 3 CDR regions of the light chain refer to LCDR1, LCDR2, and LCDR3; the 3 CDR regions of the heavy chain refer to HCDR1, HCDR2 and HCDR3.

In light chains and heavy chains, the variable region and constant region are linked by a “J” region of about 12 or more amino acids, and the heavy chain further comprises a “D” region of about 3 or more amino acids. Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL). The light chain constant region consists of one domain CL. The constant region of the antibody can mediate the binding of immunoglobulins to host tissues or factors, including the binding of various cells of the immune system (e.g., effector cells) to the first component (C1q) of classical complement system. The VH and VL regions can be further subdivided into hypervariable regions (called complementarity determining regions (CDRs)), between which conservative regions called framework regions (FWRs) are distributed. Each VH and VL consists of 3 CDRs and 4 FWRs arranged from the amino-terminus to the carboxy-terminus in the following order: FWR1, CDR1, FWR2, CDR2, FWR3, CDR3, and FWR4. The corresponding variable regions (VH and VL) of each heavy chain/light chain form an antibody-binding site, respectively. In particular, the heavy chain may also comprise 3 or more CDRs, such as 6, 9, or 12 CDRs. For example, in the bispecific antibody of the present invention, the heavy chain may be a ScFv with the N-terminus of the heavy chain of IgG antibody linked to another antibody, and in this case, the heavy chain comprises 9 CDRs.

The term “human antibody” includes antibodies having variable and constant regions of human germline immunoglobulin sequences. The human antibody of the present invention may include amino acid residues not encoded by human germline immunoglobulin sequences (e.g., mutations introduced by in vitro random or site-directed mutagenesis or in vivo somatic mutation). However, the term “human antibody” does not include antibodies in which CDR sequences derived from the germline of another mammalian species, such as a mouse, have been grafted into human framework sequences (i.e., “humanized antibodies”).

As used herein, the term “specific” with respect to an antibody means that an antibody recognizes a specific antigen but does not substantially recognize or bind to other molecules in a sample. For example, an antibody that specifically binds to an antigen from one species may also bind to the antigen from one or more species. However, such interspecies cross-reactivity per se does not change the classification of antibodies by specificity. In another example, an antibody that specifically binds to an antigen may also bind to the antigen in different allelic forms. However, such cross-reactivity per se does not change the classification of antibodies by specificity. In some cases, the term “specificity” or “specific binding” may be used to refer to the interaction of an antibody, a protein or a peptide with a second chemical, meaning that the interaction is dependent on the presence of a particular structure (e.g., an antigenic determinant or epitope) in the chemical; for example, an antibody generally recognizes and binds to a particular protein structure rather than a protein. If an antibody is specific to an epitope “A”, then in a reaction containing labeled “A” and the antibody, the presence of a molecule containing the epitope A (or free, unlabeled A) will reduce the amount of labeled A bound to by the antibody.

The term “chimeric antigen receptor” or “CAR” used herein includes extracellular domains (extracellular binding domains), hinge domains, transmembrane domains (transmembrane regions) capable of binding to antigens and polypeptides that causes passes a cytoplasmic signal to a domain (i.e., an intracellular signal domain). The hinge domain may be considered as a part for providing flexibility to an extracellular antigen-binding region. The intracellular signal domain refers to a protein that transmits information into a cell via a determined signaling pathway by generating a second messenger to regulate the activity of the cell, or a protein that functions as an effector by corresponding to such a messenger. It generates a signal that can promote the immune effector function of a cell of the CAR (e.g., a CART cell). The intracellular signal domain includes a signaling domain, and may also include a co-stimulatory intracellular domain derived from a co-stimulatory molecule.

“Homology”, “variant sequence” or “mutation” refers to sequence similarity between two polynucleotide sequences or between two polypeptide sequences. When positions in two compared sequences are all occupied by the same base or amino acid monomer subunit, for example, if a position in each of two DNA molecules is occupied by adenine, the molecules are homologous at that position. The identity percentage between two sequences is a function of the number of matched or homologous positions shared by the two sequences divided by the number of the compared positions×100%. For example, when sequences are optimally aligned, if 6 out of 10 positions in two sequences match or are homologous, the two sequences are 60% homologous. In general, the comparison is made when two aligned sequences give the greatest identity percentage. The “optimization” refers to a mutation that maintains or improves the binding of an antibody to an antigen. In the present invention, it refers to a mutation that maintains, preserves or improves the binding to CLDN18.2.

The terms “polypeptide”, “peptide” and “protein” (if single-stranded) are used interchangeably in the present invention. The terms “nucleic acid”, “nucleic acid sequence”, “nucleotide sequence” or “polynucleotide sequence” and “polynucleotide” are used interchangeably.

The term “mutation” includes substitutions, additions and/or deletions of amino acids or nucleotides. The “amino acid substitution” and “conservative amino acid substitution” are replacement of an amino acid residue by another amino acid residue and replacement by an amino acid residue with similar side chains, respectively.

The “lentivirus” used herein refers to a genus of the Retroviridae family. Lentiviruses are unique among retroviruses and are capable of infecting non-dividing cells; they are capable of delivering a considerable amount of genetic information into the DNA of a host cell, and are thus one of the most efficient methods of gene delivery vectors. HIV, SIV and FIV are all examples of lentiviruses. Vectors derived from lentiviruses provide a means to achieve significant horizontal gene transfer in vivo.

The term “vector” used herein is a composition that comprises an isolated nucleic acid and is useful for delivering the isolated nucleic acid to the interior of a cell. Many vectors are known in the art. They include, but are not limited to, linear polynucleotides, polynucleotides associated with ionic or amphiphilic compounds, plasmids, and viruses. Thus, the term “vector” includes autonomously replicating plasmids or viruses. The term should also be construed as including non-plasmid and non-viral compounds that facilitate the transfer of nucleic acids into cells, such as polylysine compounds and liposomes. Examples of viral vectors include, but are not limited to, adenoviral vectors, adeno-associated viral vectors, retroviral vectors, etc.

The expressions “cell” and “cell line” used in the present invention are used interchangeably and all such designations include progeny. The term “host cell” refers to a cell to which a vector can be introduced, including, but not limited to, prokaryotic cells such as E. coli, fungal cells such as yeast cells, or animal cells such as fibroblasts, CHO cells, COS cells, NSO cells, HeLa cells, BHK cells, HEK 293 cells, or human cells.

The term “transfection” refers to the introduction of an exogenous nucleic acid into a eukaryotic cell. Transfection may be accomplished by a variety of means known in the art, including calcium phosphate-DNA co-precipitation, DEAE-dextran mediated transfection, polybrene-mediated transfection, electroporation, microinjection, liposome fusion, lipofection, protoplast fusion, retroviral infection, and biolistics.

The term “immune cell” refers to a cell that can elicit an immune response. The “immune cell” and other grammatical variations thereof may refer to an immune cell of any origin. The “immune cell” includes, for example, white blood cells (leukocytes) and lymphocytes (T cells, B cells, and natural killer (NK) cells) derived from hematopoietic stem cells (HSCs) produced in the bone marrow, and bone marrow-derived cells (neutrophils, eosinophils, basophils, monocytes, macrophages, dendritic cells). The term “immune cell” may also refers to a human or non-human immune cell.

As used herein, the term “T cell” refers to a class of lymphocytes that mature in the thymus. T cells play an important role in cell-mediated immunity and are different from other lymphocytes (e.g., B cells) in that T cell receptors are present on the cell surface. The “T cell” includes all types of immune cells that express CD3, including T helper cells (CD4+ cells), cytotoxic T cells (CD8+ cells), natural killer T cells, T regulatory cells (Tregs), and γ-δT cells. The “cytotoxic cells” include CD8+ T cells, natural killer (NK) cells and neutrophils, which are capable of mediating a cytotoxic response. As used herein, the term “NK cell” refers to a class of lymphocytes that originate in the bone marrow and play an important role in the innate immune system. NK cells provide a rapid immune response against virus-infected cells, tumor cells or other stressed cells, even in the absence of antibodies and major histocompatibility complexes on the cell surface.

For example, the immune cells may be derived from the blood, such as autologous T cells, allogeneic T cells, autologous NK cells and allogeneic NK cells, or from cell lines, such as NK cell lines prepared by infection with the EBV virus, NK cells obtained by induced differentiation of embryonic stem cells and iPSCs, as well as NK92 cell lines.

The term “optional”, “optionally”, “any” or “any one of” means that the event or circumstance subsequently described may, but not necessarily, occur, and that the description includes instances where the event or circumstance occurs or does not occur. For example, “optionally comprising 1 antibody heavy chain variable region” means that the antibody heavy chain variable region of a particular sequence may, but not necessarily, be present. As used herein, the “a” and “an” are used in the present invention to refer to one or more grammatical objects. Unless otherwise specifically stated in the content, the term “or” is used in the present invention to mean, and is interchangeable with, the term “and/or”. The “about” and “approximately” shall generally mean an acceptable degree of error in the measured quantity in view of the nature or accuracy of the measurement. Exemplary degrees of error are typically within 10% thereof and more typically within 5% thereof. The method and composition disclosed in the present invention encompass polypeptides and nucleic acids having a specified sequence, a variant sequence, or a sequence substantially identical or similar thereto, e.g., a sequence that is at least 85%, 90%, 95%, 99% or more identical to the sequence specified. In the context of amino acid sequences, the term “substantially identical” is used in the present invention to refer to a first amino acid sequence.

As used herein, the term EC50 refers to the concentration for 50% of maximal effect, i.e., the concentration that can cause 50% of the maximal effect.

As used herein, the terms “antibody-drug conjugate” and “ADC” are used interchangeably.

Auristatin is a fully synthetic drug whose chemical structural formula is relatively easy to modify so as to optimize its physical properties and druggability. Auristatin derivatives used for conjugation with antibodies mainly include monomethyl auristatin E (MMAE) and monomethyl auristatin F (MMAF), wherein the former is synthesized by adding 2-amino-1-phenylpropyl-1-ol to the C-terminus of a synthetic pentapeptide derived from a natural tubulin polymerase inhibitor dolastatin-10. The inhibitory activity of MMAE against various human tumor cell lines is less than one nanomole. In order to reduce the cytotoxic activity of MMAE, a phenylalanine is added to the C-terminus of dolastatin-10 in MMAF. The cell membrane trafficability of MMAF is poor for the carboxyl introduced in the structure. Therefore, the bioactivity for cells is significantly reduced, but the inhibitory activity against cells is greatly improved after conjugation with an antibody (U.S. Pat. No. 7,750,116).

In some embodiments, an antibody-cytotoxic drug conjugate or a pharmaceutically acceptable salt or solvate thereof comprises the antibody of the present invention conjugated with one or more maytansinoid molecules. Maytansinoids are mitotic inhibitors that disable tubulin by inhibiting polymerization of tubulin. Maytansine was originally isolated from the East African shrub Maytenus serrata (U.S. Pat. No. 3,896,111). It was subsequently discovered that certain microorganisms also produce maytansinoids, such as maytansinol and C-3 maytansinol vinegar (U.S. Pat. No. 4,151,042). Maytansinoid drug modules are appealing drug modules in antibody-drug conjugates as they are: (i) relatively easy to prepare by fermentation or chemical modification or derivatization of the fermentation product; (ii) readily derivatized with functional groups suitable for conjugation to antibodies through non-disulfide linkers; (iii) stable in plasma; and (iv) effective for a variety of tumor cell lines. Maytansine compounds suitable for use as maytansinoid drug modules are well known in the art and can be isolated from natural sources according to known methods or produced using genetic engineering techniques (see Yu et al. (2002) PNAS 99:7968-7973). Maytansinol and maytansinol analogues can also be prepared synthetically according to known methods. Exemplary embodiments of maytansinoid drug modules include: DM1, DM3 and DM4, as disclosed herein.

The method, composition and combination therapy of the present invention may be combined with other active agents or therapeutic modalities, the method comprising administering to a subject the anti-CLDN18.2 antibody molecule of the present invention in an amount that is effective in treating or preventing diseases (e.g., cancer), optionally in combination with one or more inhibitors of PD-1, PD-L1, PD-L2, LAG-3, CTLA-4, Tim-3 antibody (immunotherapy) or other tumor therapy antibodies, Her-2, EGFR, VEGF, VEGFR antibodies, etc., as well as ADCs (e.g., T-DM1), bispecific antibodies, chemotherapeutic agents, etc., and further comprising administering an anti-CLDN18.2 antibody molecule, an additional active agent or all in an amount or at a dose that is higher, lower or equal to the amount or dose for each active agent when used alone (e.g., as a monotherapy). The amount or dose for the anti-CLDN18.2 antibody, the additional active agent or all administered is, e.g., at least 20%, at least 30%, at least 40% or at least 50%, lower than the amount or dose for each active agent when used alone (e.g., as a monotherapy).

Furthermore, as described in the examples of the present invention, the anti-CLDN18.2 antibody and the drug conjugate of the CLDN18.2 antibody can bind to CLDN18.2 to induce apoptosis of target cells (tumor cells), to inhibit the growth of tumor cells, and to increase the effector cells' ADCC and CDC killing effects on tumor cells in vivo, thereby achieving the purpose of treating cancer patients. Thus, in certain embodiments, the anti-CLDN18.2 antibody and drug conjugate of the CLDN18.2 antibody described in the present invention show the anti-tumor effects of the antibody of the present invention, as well as methods for inhibiting the growth of tumor cells comprising administering to a subject a therapeutically effective amount of the anti-CLDN18.2 antibody and drug conjugate of the CLDN18.2 antibody of the present invention, through these mechanisms. The method is suitable for the in vivo treatment of cancer. To achieve a targeted specific therapeutic effect, the anti-CLDN18.2 antibody molecule may be administered together with other antibodies. In administering the CLDN18.2 antibody and the drug conjugate of the CLDN18.2 antibody in combination with one or more active agents, the combination may be administered in any order or simultaneously to a patient with a type of cancer, particularly a tumor patient with high expression of CLDN18.2. In certain aspects, treatment (e.g., reduction or amelioration) of a hyperproliferative symptom or disease (e.g., cancer) in a subject is provided. The method comprises administering to a subject one or more anti-CLDN18.2 antibodies or drug conjugates of the CLDN18.2 antibodies of the present invention, either alone or in combination with other active agents or therapeutic modalities.

The anti-CLDN18.2 antibody molecule, alone or in combination with another immunomodulatory agent (e.g., anti-LAG-3, anti-Tim-3, anti-PD-L or anti-PD-L1, and anti-CTLA-4 antibody molecules), is employed to treat gastric cancer, pancreatic cancer, lung cancer, esophageal cancer, ovarian cancer, etc. The anti-CLDN18.2 antibody molecule may be administered in combination with one or more of: an immunity-based strategy, a targeted drug (e.g., a VEGF inhibitor such as a monoclonal antibody against VEGF); VEGF tyrosine kinase inhibitors such as sunitinib, sorafenib and apatinib; RNAi inhibitors or inhibitors of downstream mediators of the VEGF signaling, e.g., inhibitors of rapamycin mammalian target (mTOR).

As used herein, the terms “cancer,” “cancer patient” are intended to include all types of cancerous growths or oncogenic processes, metastatic tissues or malignantly transformed cells, tissues or organs, regardless of their histopathological types or stages of invasiveness. Examples include, but are not limited to, solid tumors, hematologic cancer, soft tissue tumors and metastatic lesions.

Non-limiting examples of cancers that may be suitably treated using the CLDN18.2-targeting antibody disclosed in the present invention include gastric cancer, esophageal cancer, lung cancer, melanoma, renal cancer, breast cancer, colorectal cancer, liver cancer, pancreatic cancer, bladder cancer, glioma and/or leukemia and the like, or metastatic lesions thereof.

The above preferred conditions may be combined arbitrarily to obtain preferred embodiments of the present invention on the basis of the general knowledge in the art.

The reagents and starting materials used in the present invention are commercially available.

The beneficial effects of the present invention are as follows:

1. The present invention describes anti-CLDN18.2 HCAb antibodies with excellent affinity, specificity and endocytic activity. The antibody is a brand-new fully human antibody containing a “heavy chain” only with a molecular weight that is only about half of that of a conventional IgG antibody. Due to the absence of a light chain, the antibody can be used for the development of bispecific antibodies, and the common problems of light chain mismatching and heterodimerization in the development of bispecific antibodies are avoided. It also has the potential to be developed into ADCs. In certain preferred embodiments, the HCAb antibody has great affinity for tumor cells endogenously expressing CLDN18.2 and can induce great endocytic activity compared to an IMAB362 analogue.

2. The present invention also describes CLDN18.2×CD3 bispecific antibodies with excellent in vitro TDCC activity and in vivo drug effect. The bispecific antibody of the present invention has the activity of specifically binding to CLDN18.2, and has a better killing effect on tumor cells than a patent bispecific antibody analogue of Amgen. In certain preferred embodiments, the bispecific antibody has a Fc fragment and thus retains the binding effect of the Fc to an FcRn; meanwhile, a mutant Fc is preferred so as to reduce the binding to an FcgR and thus the activation of non-specific T cells caused by the cross-linking of an FcgR. The CD3-termini activity is optimized so that the release of common cytokines in CRS, such as IL6 and TNFα can be reduced. The CLDN18.2 termini is in the form of VHHs linked in series, avoiding the common problem of mismatching of light and heavy chains, retaining the excellent hydrophilicity and improving the selectivity for tumor cells with high expression of CLDN18.2. The antibody has good in vivo stability and long in vivo half-life and shows great in vivo anti-tumor activity.

### BRIEF DESCRIPTION OF THE DRAWINGS

FIGS. 1a-f show the binding affinity of HCAB antibodies for (a-b) NUGC4_D8, (c) SNU601, (d) HEK293/hCLDN18.2, and (e-f) HEK293/hCLDN18.1 cells.

FIGS. 2a-b show the competitive binding activity of HCAB antibodies against (a) PR000400 and (b) PR004549 for HEK293/hCLDN18.2 cells.

FIGS. 3a-b show the viability of target cells when co-cultured with test antibodies and an MMAF-coupled anti-human IgG antibody.

FIGS. 4a-h show the structures of CLDN18.2×CD3 bispecific antibodies.

FIGS. 5a-s show the binding affinity of CLDN18.2×CD3 bispecific antibodies for (a-c) HEK293/hCLDN18.2, (d-i) NUGC4_D8, (j-o) Jurkat, and (p-s) HEK293/hCLDN18.1 cells.

FIGS. 6a-w show (a) the expression yield of CLDN18.2 by IM95 cells, and the TDCC activity of bispecific antibodies against (b-k) NUGC4_D8, (1-0) IM95, (p-s) HEK293/hCLDN18.1, and (t-w) SNU620 cells.

FIG. 7 shows the binding affinity of CLDN18.2×CD3 bispecific antibodies for cells overexpressing human CLDN18.2 paralogous family protein CLDN1, CLDN2, CLDN3, CLDN4, CLDN6 and CLDN9.

FIGS. 8a-c show the binding affinity of CLDN18.2×CD3 bispecific antibodies for (a) HEK293/cynoCLDN18.1, (b) HEK293/cynoCLDN18.2, and (c) cynomolgus CD3+ T cells.

FIG. 9 shows the TDCC activity of CLDN18.2×CD3 bispecific antibodies against HEK293/cynoCLDN18.2 cells by cyno T cells.

FIGS. 10a-b show the release of cytokines induced by CLDN18.2×CD3 bispecific antibodies in vitro. (a) TNFα, and (b) IL-6.

FIGS. 11a-b show the ADCC activity of CLDN18.2×CD3 bispecific antibodies against (a) Jurkat and (b) NUGC4_D8 cells.

FIGS. 12a-b show the CDC activity of CLDN18.2×CD3 bispecific antibodies against (a) HEK293/hCLDN18.2 and (b) Jurkat cells.

FIGS. 13a-b show the competitive binding activity of CLDN18.2×CD3 bispecific antibodies against (a) PR000400 and (b) PR004549 for HEK293/hCLDN18.2 cells.

FIG. 14 shows the pharmacokinetics of CLDN18.2×CD3 bispecific antibodies.

FIGS. 15a-e show in vivo pharmacodynamics studies in (a-b) NUGC4_D8 PBMC, (c) SNU620 PBMC, and (d) HuP-T4 PBMC tumor models and (e) in vivo cytokine storm study on CLDN18.2×CD3 bispecific antibodies.

### DETAILED DESCRIPTION

The present invention is further illustrated by the following examples, which are not intended to limit the present invention. Experimental procedures without specified conditions in the following examples are performed in accordance with conventional procedures and conditions, or in accordance with instructions.

### Example 1: Preparation of Expression Vector and Stably Transfected Cell Lines and Immunization of Mice

### 1.1. Preparation of Expression Vector for Immunizing Mice

A human CLDN18.2 expression vector for immunizing fully humanized transgenic mice was prepared as follows: a cDNA sequence encoding human CLDN18.2 (Uniprot ID P56856-iso2) was synthesized, and the coding sequence of the human CLDN18.2 gene was cloned into a pCAGGS plasmid (YOUBIO, VT1076) by enzymatic digestion.

### 1.2. Preparation of Stably Transfected Cell Lines

HEK293 (ATCC, Cat #: CRL-1573) cell lines stably expressing human CLDN18.1 or CLDN18.2 were constructed specifically as follows: plasmids encoding human CLDN18.1 (GenScript, OHu29174D) or CLDN18.2 (GenScript, OHu03374D) were transfected into HEK293 cells to produce stable cell lines overexpressing human CLDN18.1 or CLDN18.2. The expression of CLDN18.1 and CLDN18.2 was detected by fluorescence activated cell sorting (FACS). Specifically, 20,000 transfected cells were plated in each well of a 96-well plate, followed by the addition of a commercially-available rabbit anti-human CLDN18 antibody (LifeSpan Bio, LS-C168812-400). After 1 h of incubation at 4° C., the cells were washed 2 times with PBS, and then an AF-680-conjugated secondary goat anti-rabbit IgG antibody (Invitrogen, A21109) was added. After 1 h of incubation at 4° C., the cells were washed 3 times with PBS, and then the fluorescence intensity of the cells was monitored using an FACS instrument (IntelliCytiQue Plus BR).

### 1.3. Immunization of Mice

Fully humanized transgenic mice (commercially available Harbour HCAB 1.0 mice, purchased from Harbour BioMed) were immunized with the human CLDN18.2 expression vector and CLDN18.2-expressing HEK293 cells (HEK293/hCLDN18.2 cells) prepared above. Bullets for a gene gun were prepared with the human CLDN18.2 expression vector and gold powder. Mice were immunized at multiple sites of the abdomen using the gene gun. The mice were immunized with the expression vector DNA (50 μg each time) at 2-week intervals. After three immunizations, the mice were then immunized with HEK293/hCLDN18.2 cells at 2-week intervals, with 4×10⁶ cells per mouse for each immunization. After two immunizations, the blood was collected for titer measurement. The mouse serum was assayed for binding affinity by FACS using human CLDN18.2-expressing CHOK1 cells (kyinno, KC-1180). Mice were selected according to the results of the titer measurement for screening of HCAB monoclonal antibodies. The mice were subjected to boosted immunization 3 days prior to the screening, with HEK293/hCLDN18.2 cells, 4×10⁶ cells per mouse, as immunogen-.

### Example 2: Production and Screening of Anti-CLDN18.2 HCAB Single-Domain Antibodies

The mice with high anti-CLDN18.2 antibody serum titer obtained in Example 1 were selected. The spleens of these mice were collected, and B cells were isolated. CD138 (BD, 558626) positive plasma cells were sorted using a BD FACS AriaIII cell sorter, and CLDN18.2 (CHOK1/hCLDN18.2, kyinno, KC-1180) positive B Cell populations were enriched using magnetic beads (Thermofisher, 11206D). The RNA of the B cells was extracted and reverse transcribed into cDNA (SuperScript IV First-Strand synthesis system, Invitrogen, 18091200), and human VH genes were amplified by PCR using specific primers. PCR primers:

(SEQ ID NO: 249)
5′-GGTGTCCAGTGTSAGGTGCAGCTG-3′
(SEQ ID NO: 250)
5′-AATCCCTGGGCACTGAAGAGACGGTGACC-3′

The amplified VH gene fragments were constructed into mammalian cell expression plasmid pCAG vectors encoding the sequence of heavy chain Fc domain of human IgG1 antibody.

The constructed plasmids were transfected into HEK293 mammalian host cells (ATCC, CRL-1573) to obtain an expression supernatant of the HCAb antibody. Primary screening was performed by Mirrorball using CHOK1/hCLDN18.2 expressing human CLDN18.2. Positive clones were selected for secondary screening. The secondary screening was performed by FACS using HEK293/hCLDN18.1 and HEK293/hCLDN18.2 cells.

While 295 monoclonal antibodies specifically binding to human CLDN18.2 were obtained, the nucleotide sequences encoding the variable domains of the antibody molecules and the corresponding amino acid sequences were obtained using conventional sequencing means. After the removal of repeated sequences, 211 fully human CLDN18.2 HCAb monoclonal antibodies having unique sequences and specifically binding to human CLDN18.2 were obtained. 54 antibodies with top comprehensive ranking were selected for recombinant expression according to the primary screening result and the secondary screening result. The purified monoclonal antibodies were further screened for binding ability to tumor cells endogenously expressing human CLDN18.2 by flow cytometry, and the top 8 antibody sequences in the comprehensive rankings were selected as candidate molecules, as shown in Table 3.

It is well known to those skilled in the art that the CDRs of an antibody can be defined in the art using a variety of methods, such as the Kabat scheme based on sequence variability (see Kabat et al., Sequences of Proteins of Immunological Interest, Fifth Edition, National Institutes of Health (U.S.), Bethesda, Maryland (1991)), and the Chothia scheme based on the location of the structural loop regions (see J Mol Biol 273:927-48, 1997). In the present application, the Combined scheme comprising the Kabat scheme and the Chothia scheme can also be used to determine the amino acid residues in a variable domain sequence. The Combined scheme combines the Kabat scheme with the Chothia scheme to obtain a larger range, which is detailed in Table 1 of the summary of the present invention. The germline gene analysis and the PTM site analysis obtained after sequencing from this example are shown in Table 2 below. The sequence numbers of the antigen-binding proteins are shown in Table 3 below.

TABLE 2
Germline gene analysis and post-translational modification
site (PTM) analysis of sequences of HCAb antibodies

| | | | VH germline V | |
|---|---|---|---|---|
| No. | Clone No. | Antibody | gene | VH PTM |
| 1 | R1029P(CG20)001B02 | PR004227 | IGHV3-74*01 | DG (HCDR2) |
| 2 | R1029P021E10 | PR004533 | IGHV3-74*03 | NS (HCDR2), DG (HCDR2) |
| 3 | R1029P028A11 | PR004536 | IGHV3-74*01 | NS (HCDR2), DG (HCDR2) |
| 4 | R1029P037B07 | PR004540 | IGHV3-74*01 | DG (HCDR2), NxS/T (HCDR2) |
| 5 | R1029P021C02 | PR004949 | IGHV3-74*03 | NS (HCDR2), DG (HCDR2) |
| 6 | R1029P021F07 | PR004950 | IGHV3-74*01 | NS (HCDR2), DG (HCDR2) |
| 7 | R1029P021H07 | PR004952 | IGHV3-74*03 | NS (HCDR2), DG (HCDR2) |
| 8 | R1029P024B07 | PR004953 | IGHV3-30*03 | DG (HCDR2) |

TABLE 3
Sequence numbers of anti-CLDN18.2 HCAb

SEQ ID NOs:

| | Heavy | | | | |
|---|---|---|---|---|---|
| Antibody | chain | VH | HCDR1 | HCDR2 | HCDR3 |
| PR004227 | 182 | 150 | 16 | 42 | 77 |
| PR004533 | 183 | 151 | 16 | 43 | 78 |
| PR004536 | 184 | 152 | 16 | 44 | 79 |
| PR004540 | 185 | 153 | 17 | 45 | 80 |
| PR004949 | 186 | 154 | 18 | 43 | 80 |
| PR004950 | 187 | 155 | 18 | 43 | 78 |
| PR004952 | 188 | 156 | 16 | 43 | 81 |
| PR004953 | 189 | 157 | 16 | 46 | 82 |

### Example 3: Removal of Post-Translational Modification Site of Anti-CLDN18.2 HCAB Antibodies

PR004533 and PR004536 both have an isomerization site and a deamidation site in the CDR2 region of the heavy chain. For the post-translational modification site in the CDR2 region, four amino acids of NS and DG were randomly mutated by PCR. The PCR products were electrically transfected into E. coli to establish a random mutation library of four amino acid sites. The mutant library was screened by Mirrorball using CHOK1/hCLDN18.1 and CHOK1/hCLDN18.2 cells, and positive molecules that specifically bind to CLDN18.2 were selected for sequencing. The sequence numbers of the molecules are shown in Table 4 below.

TABLE 4
Part of the sequence numbers of PR004533
and PR004536 mutant molecules

| | Heavy | | | | | |
|---|---|---|---|---|---|---|
| Antibody | chain | VH | HCDR1 | HCDR2 | HCDR3 | Mutant clone |
| PR007242 | 191 | 159 | 16 | 48 | 78 | 4533_M10E1 |
| PR007243 | 192 | 160 | 16 | 49 | 78 | 4533_M3D9 |
| PR007244 | 193 | 161 | 16 | 50 | 78 | 4533_M4H6 |
| PR007245 | 194 | 162 | 16 | 51 | 78 | 4533_M6E6 |
| PR007246 | 195 | 163 | 16 | 52 | 78 | 4533_M3F10 |
| PR007247 | 196 | 164 | 16 | 53 | 79 | 4536_M8A6 |
| PR007248 | 197 | 165 | 16 | 54 | 78 | 4533_M10E7 |

### Example 4: Preparation and Characterization of Full-Length Anti-CLDN18.2 HCAb Single-Domain Antibodies

### 4.1. Preparation of Recombinant HCAb Single-Domain Antibodies

After obtaining the sequence of the heavy chain variable domain encoding the HCAb single-domain antibody molecules, the sequence of the heavy chain variable domain can be fused with the corresponding sequence of the heavy chain constant domain of the human antibody and expressed using conventional recombinant DNA techniques to obtain recombinant HCAb single-domain antibody molecules. In this example, the sequence of the heavy chain variable domain (VH) of the antibody was genetically synthesized and cloned into a mammalian cell expression plasmid vector encoding the sequence of the heavy chain constant domain of the human IgG1 antibody to encode a full-length sequence producing the HCAb single-domain antibody. In this example, the sequences of the variable domains of the monoclonal antibody molecules obtained from immunized Harbour HCAb mice were human antibody sequences, therefore, a fully human anti-CLDN18.2 recombinant HCAb antibody was also obtained from this example.

The plasmids encoding recombinant HCAb single-domain antibodies were transfected into mammalian host cells (e.g., Chinese Hamster Ovary (CHO) cells), and the corresponding purified recombinant antibody can be obtained using conventional recombinant protein expression and purification techniques. Specifically, ExpiCHO-S™ cells (Gibco, A29127) were expanded in ExpiCHO™ Expression Medium (Gibco, A2910001). Before the transient transfection, the cells were adjusted to a concentration of 3×10⁶ to 4×10⁶ cells/mL, and cultured in an 8% CO₂ shaker at 37° C. for 24 h, leading to a cell concentration of 7×10⁶ to 10×10⁶ cells/mL. The cells were then diluted to 6×10⁶ cells/mL, and 10 mL of the cultured cells was prepared. 8 μg of the above plasmids encoding HCAb single-domain antibodies (the ratio of the plasmids to cells is 0.8 μg:1 mL) was dissolved in 0.4 mL of OptiPRO™ SFM medium (Gibco, 12309019). The resulting mixture was filtered through a 0.22 μm filter for sterilization. Then 32 μL of ExpiFectamine™ CHO reagent (Gibco, A29129) was added to 0.37 mL of OptiPRO™ SFM medium (Gibco, 12309019). The ExpiFectamine™ CHO reagent solution was immediately added slowly to the plasmid solution. The mixture was inverted to be well mixed. The mixed solution of plasmid and transfection reagent was slowly added dropwise while shaking the flask. The cells were cultured in an 8% CO₂ shaker at 37° C. for 8-9 days. The Cell viability was observed after 8 days.

The culture was collected and centrifuged at 3,300 g for 10 min, and then the supernatant was collected and centrifuged at high speed to remove impurities. A gravity column (Bio-Rad, #7311550) containing MabSelect™ (GE Healthcare Life Science, 71-5020-91 AE) was equilibrated with PBS (pH 7.4) and rinsed with 2-5 column volumes of PBS. The supernatant sample was loaded onto a column. The column was rinsed with 5-10 column volumes of PBS. The target protein was eluted with 0.1 M glycine (pH 3.5). The eluate was adjusted to neutrality with Tris-HCl (pH 8.0), and concentrated and buffer exchanged into PBS buffer with an ultrafiltration tube (Millipore, UFC901024) to obtain a purified antibody solution. Then, the purified antibody solution was subjected to concentration determination using NanoDrop (Thermo Scientific™ NanoDrop™ One), subpackaged and stored for later use.

### 4.2. Antibody Characterization by SEC-HPLC, HIC-HPLC and DSF

A proper amount of the purified sample above was loaded onto an analytical SEC column TSKgel G3000SWxl (HPLC system model: Agilent 1260 Infinity II) for the measurement of purity. In this method, the following parameters and conditions were used: mobile phase: 1×PBS, pH 7.4 (Sangon, E607016); room temperature; flow rate: 1.0 mL/min; sample concentration: 1 mg/mL; injection volume: 20 μL; detection wavelength: 280 nm. After being recorded, the chromatogram was integrated using ChemStation software and relevant data were calculated. An analysis was generated, with the retention times reported for different components in the sample.

A proper amount of the purified sample above was loaded onto an analytical HIC column TSKgel Butyl-NPR 4.6*35 (HPLC system model: Agilent 1260 Infinity II) for the measurement of purity and hydrophobicity. The method consisted of a linear gradient from 100% mobile phase A (20 mM PB, 1.8 M (NH4)₂SO₄, pH 6.0) to 100% mobile phase B (20 mM PB, pH 6.0) within 16 min. The flow rate was set at 0.7 mL/min, the sample concentration was 1 mg/mL, the injection volume was 20 μL, and the detection wavelength was 280 nm. After being recorded, the chromatogram was integrated using ChemStation software and relevant data were calculated. An analysis was generated, with the retention times reported for different components in the sample.

In this example, the thermal denaturation temperature (Tm) of a protein molecule was measured by differential scanning fluorimetry (DSF). 10 μg of protein was added to a 96-well PCR plate (Thermo, AB-0700/W), followed by the addition of 2 μL of 100× diluted dye SYPRO™ (Invitrogen, 2008138), and then buffer was added to give a final volume of 40 μL per well. The PCR plate was sealed, placed in a real-time fluorescent quantitative PCR instrument (Bio-Rad CFX96 PCR System), incubated at 25° C. for 5 min, then gradually warmed from 25° C. to 95° C. at a gradient of 0.2° C./0.2 min, and cooled to 25° C. at the end of the test. The FRET scanning mode was used and data analysis was performed using Bio-Rad CFX Maestro software to calculate the Tm of the sample. The results of the above characterization are shown in Table 5 below.

TABLE 5
Characterization of anti-CLDN18.2 HCAB antibodies

| | | | HIC-HPLC retention | | |
|---|---|---|---|---|---|
| | SEC-HPLC purity | HIC-HPLC purity | time | Tm1 | Tm2 |
| Antibody | (%) | (%) | (min) | (° C.) | (° C.) |
| PR004227 | 87.8 | 86.5 | 19.2 | 52.2 | 64.4 |
| PR004533 | 99.2 | 100.0 | 17.2 | NA | 64.2 |
| PR004536 | 97.6 | 100.0 | 17.1 | 52.8 | 66.0 |
| PR004540 | 99.4 | 100.0 | 17.5 | 52.8 | 65.4 |
| PR004949 | 99.2 | 100.0 | 16.0 | 59.0 | NA |
| PR004950 | 98.7 | 100.0 | 16.2 | NA | 65.4 |
| PR004952 | 100.0 | 100.0 | 15.8 | 61.2 | NA |
| PR004953 | 98.2 | 100.0 | 18.9 | 60.0 | NA |
| PR007242 | 94.3 | 100.0 | 15.7 | 56.6 | 65.4 |
| PR007243 | 99.1 | 92.5 | 15.3 | 55.6 | 64.8 |
| PR007244 | 99.1 | 94.3 | 15.3 | 59.6 | NA |
| PR007245 | 98.9 | 94.5 | 15.2 | 59.8 | NA |
| PR007246 | 99.1 | 93.9 | 15.2 | 59.0 | 64.6 |
| PR007247 | 98.1 | 94.3 | 16.3 | 52.0 | 65.2 |
| PR007248 | 99.3 | 100.0 | 15.4 | 60.6 | NA |
| Trastuzumab | 100.0 | 100.0 | 16.0 | 67.8 | 79.2 |

### Example 5: Binding Affinity of Anti-CLDN18.2 HCAB Antibodies to Cells

The binding affinity of an antibody was detected by FACS using human CLDN18.1-expressing HEK293 cells, human CLDN18.2-expressing HEK293 cells, NUGC4_D8 cells and SNU601 cells endogenously expressing human CLDN18.2 (Cobioer, CBP60507). Subcloned NUGC4_D8 cells were screened by limiting dilution using NUGC4 cells (JCRB, JCRB0834). The binding affinity was determined as follows: cells were centrifuged at 300 g for 5 min and then resuspended in FACS buffer (PBS containing 2% FBS). The cell density was adjusted to 10⁶ cells/mL, and 50 μL of the cell suspension was added to each well of a 96-well plate. Antibodies were diluted to different concentrations with FACS buffer, and 50 μL of the antibody dilution was added to each well of the 96-well plate. After 2 h of incubation at 4° C., the plate was washed twice with FACS buffer. Then, FACS buffer containing an APC-conjugated goat anti-human IgG secondary antibody (Jackson, 109-605-098) was added. After 1 h of incubation at 4° C., the plate was washed twice with FACS buffer. The cells were resuspended in fixative solution, and then the fluorescence of the cells was monitored using an FACS instrument (ACEA NovoCyte). PR000400, an IMAB362 analogue (produced in-house, see WO 2014/146672, having the same variable region as IMAB362, differing from IMAB362 in only a few amino acids in the constant region), was used as a positive control for CLDN18.2 binding. PR004549, a CL-1×I2C scFc analogue (produced in-house, see WO2020025792A1, having the same variable region as CL-1×I2C scFc, differing from CL-1×I2C scFc in only a few amino acids in the constant region), was used as a positive control for CLDN18.2 binding. PR002725 antibody was used as a positive control for CLDN18.1 binding. See CN2020/118650 (Table 6, Table 7, and Table 8). Iso hIgG1 (CrownBio, C0001-4) antibody was used as a negative control.

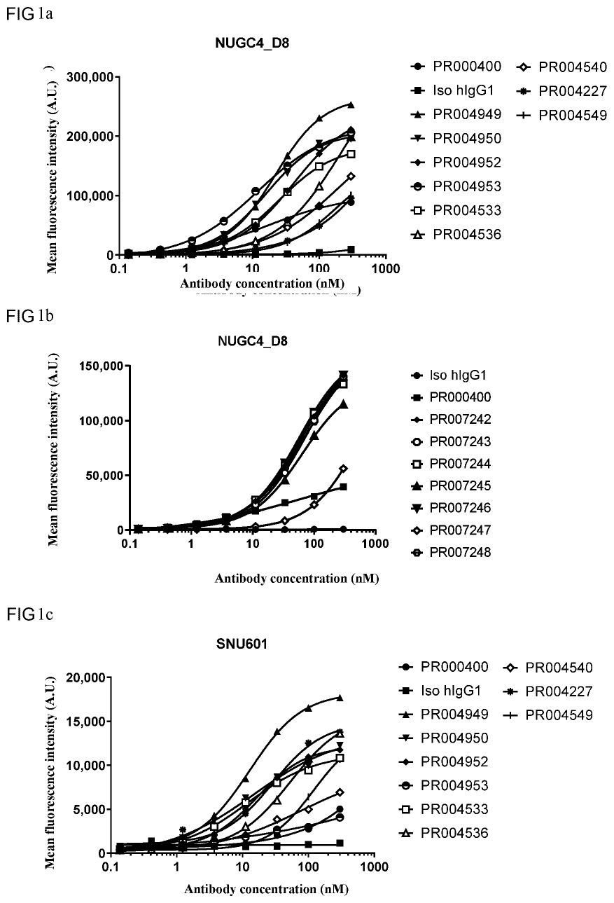

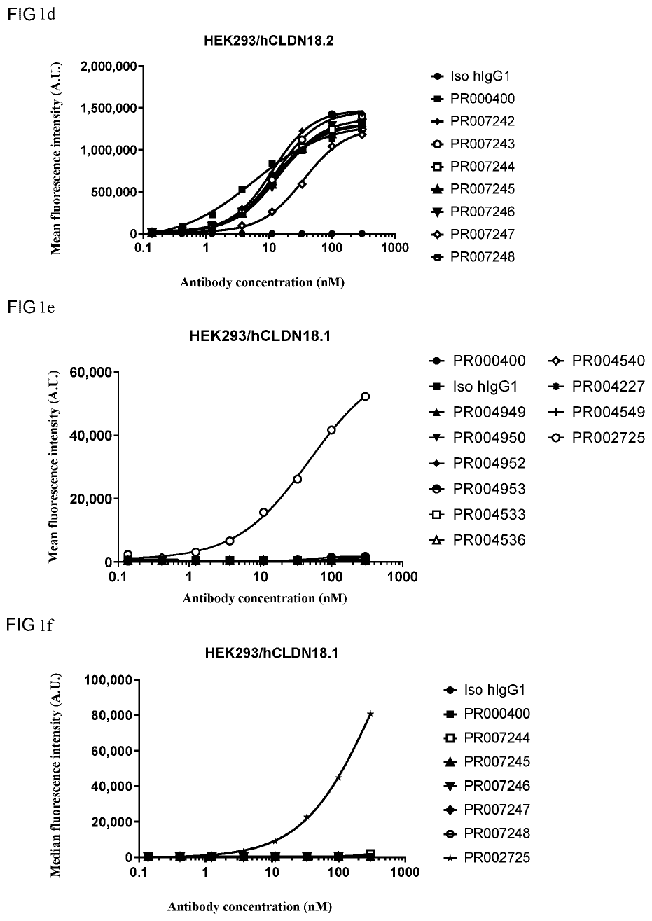

FIGS. 1 (a-b), Table 9 and Table 10 show the binding affinity of antibodies for NUGC4_D8 cells endogenously expressing CLDN18.2. The test antibodies were able to bind to NUGC4_D8 cells in a dose-dependent manner. The results show that: PR004533, PR004949, PR004950, PR004952, PR004953, PR007242, PR007243, PR007244, PR007245, PR007246 and PR007248 antibodies exhibit higher affinity for NUGC4_D8 cells endogenously expressing CLDN18.2 than PR000400. FIG. 1c and Table 11 show the binding affinity of antibodies for SNU601 cells endogenously expressing CLDN18.2. The test antibodies were able to bind to SNU601 cells in a dose-dependent manner. The results show that: PR004227, PR004533, PR004536, PR004540, PR004949, PR004950 and PR004952 antibodies exhibit higher affinity for SNU601 cells endogenously expressing CLDN18.2 than PR000400. FIG. 1d and Table 12 show the binding affinity of antibodies for HEK293 cells overexpressing human CLDN18.2 (HEK293/hCLDN18.2). FIGS. 1 (e-f) show the binding affinity of antibodies to HEK293 cells overexpressing human CLDN18.1 (HEK293/hCLDN18.1). The test antibodies have low binding affinity for HEK293/hCLDN18.1 cells. From the above results, it can be inferred that the test antibodies bind to human CLDN18.2 protein at ECL1 (Extracellular loop 1) rather than ECL2.

TABLE 6
Description for reference antibody molecules

| Antibody | Target | Description |
|---|---|---|
| PR000400 | CLDN18.2 | An anti-CLDN18.2 IgG1 monoclonal antibody with a sequence from |
| | | patent WO2014/146672. |
| PR001861 | CLDN18.1 | An anti-CLDN18.1 IgG1 monoclonal antibody, produced in-house by |
| | | Harbour BioMed. |
| PR002725 | CLDN18.1 | An anti-CLDN18.1 IgG1 monoclonal antibody with a sequence from |
| | | patent PCT/CN2020/118650. |
| PR002726 | CLDN18.2 | An anti-CLDN18.2 IgG1 monoclonal antibody with a sequence from |
| | | patent PCT/CN2020/118650. |
| PR000325 | Chicken | A chicken lysozyme-targeting IgG1 monoclonal antibody, used as an |
| | lysozyme | isotype control. |
| PR001848 | CD3 | An anti-CD3 IgG1 monoclonal antibody with a sequence from patent |
| | | PCT/CN2020/118606. |
| PR003886 | CD3 | An anti-CD3 IgG1 monoclonal antibody with a sequence from patent |
| | | PCT/CN2020/118606. |
| PR005080 | CLDN2 | An anti-CLDN2 IgG1 monoclonal antibody with a sequence from patent |
| | | EP3567053A1. |
| PR003767 | TRBC1 | An anti-TRBC1 IgG1 monoclonal antibody, used as a control for ADCC |
| | | experiments. |
| PR004549 | CLDN18.2/CD3 | A CLDN18.2 × CD3 bispecific antibody with a sequence from patent |
| | | WO2020025792A1. |
| PR004931 | CEA/CD3 | A CEA × CD3 bispecific antibody with a sequence from patent |
| | | WO2017055389A1. |
| PR002199 | BCMA/CD3 | A BCMA × CD3 bispecific antibody with a sequence from patent |
| | | WO2018052503. |
| PR004312 | Chicken | A lysozyme and CD3-targeting bispecific antibody, constructed using the |
| | lysozyme/CD3 | sequences of PR000325 andPR001848. |
| PR004313 | CLDN18.1/CD3 | A CLDN18.1 and CD3-targeting bispecific antibody, constructed using |
| | | the sequences of PR001861 and PR001848. |

TABLE 7
Sequence numbers of reference antibodies (monoclonal antibodies)

| | Light | Heavy | | | | | | | | |
|---|---|---|---|---|---|---|---|---|---|---|
| Antibody | chain | chain | VL | VH | LCDR1 | LCDR2 | LCDR3 | HCDR1 | HCDR2 | HCDR3 |
| PR000400 | 199 | 175 | 167 | 143 | 100 | 115 | 130 | 10 | 37 | 71 |
| PR001861 | 201 | 177 | 169 | 145 | 102 | 117 | 132 | 12 | 39 | 73 |
| PR002725 | 202 | 178 | 170 | 146 | 103 | 117 | 132 | 13 | 39 | 74 |
| PR002726 | 203 | 179 | 171 | 147 | 104 | 118 | 133 | 14 | 40 | 75 |
| PR000325 | 198 | 174 | 166 | 142 | 99 | 114 | 129 | 9 | 36 | 70 |
| PR001848 | 200 | 176 | 168 | 144 | 101 | 116 | 131 | 11 | 38 | 72 |
| PR003886 | 200 | 181 | 168 | 149 | 101 | 116 | 131 | 11 | 38 | 72 |
| PR005080 | 205 | 190 | 173 | 158 | 106 | 120 | 135 | 19 | 47 | 83 |
| PR003767 | 204 | 180 | 172 | 148 | 105 | 119 | 134 | 15 | 41 | 76 |

TABLE 8
Sequence numbers of reference antibodies (bispecific antibodies)

| | SEQ ID NOs: | | | |
|---|---|---|---|---|
| | Polypeptide | Polypeptide | Polypeptide | Polypeptide |
| Antibody | chain-1 | chain-2 | chain-3 | chain-4 |
| PR004549 | 212 | | | |
| PR004931 | 218 | 215 | 217 | 216 |
| PR002199 | 206 | 207 | 208 | |
| PR004312 | 210 | 209 | 200 | |
| PR004313 | 211 | 209 | 200 | |

TABLE 9
The binding affinity of anti-CLDN18.2 antibodies
(with post-translational modification sites in CDRs)
for NUGC4_D8 cells (see FIG. 1a)

| | Titer | Efficacy |
|---|---|---|
| | EC₅₀ | Eₘₐₓ |
| Antibody | (nM) | (MFI) |
| PR000400 | 17.0 | 100,612 |
| PR004227 | NA | NA |
| PR004533 | 25.0 | 183,235 |
| PR004536 | NA | NA |
| PR004540 | NA | NA |
| PR004949 | 21.8 | 265,481 |
| PR004950 | 16.1 | 206,071 |
| PR004952 | 41.0 | 239,521 |
| PR004953 | 12.0 | 218,991 |
| PR004549 | NA | NA |

TABLE 10
The binding affinity of PR004533 and
PR004536 mutant antibodies for
NUGC4_D8 cells (see FIG. 1b)

| | Titer | Efficacy |
|---|---|---|
| | EC₅₀ | Eₘₐₓ |
| Antibody | (nM) | (MFI) |
| PR000400 | 56.4 | 58,383 |
| PR007242 | 69.5 | 176,098 |
| PR007243 | 78.2 | 176,068 |
| PR007244 | 49.2 | 152,567 |
| PR007245 | 63.2 | 136,796 |
| PR007246 | 55.9 | 165,611 |
| PR007247 | NA | NA |
| PR007248 | 74.6 | 174,113 |

TABLE 11
The binding affinity of anti-CLDN18.2
antibodies for SNU601 cells

| | Titer | Efficacy |
|---|---|---|
| | EC₅₀ | Eₘₐₓ |
| Antibody | (nM) | (MFI) |
| PR000400 | NA | NA |
| PR004227 | 28.5 | 14,849 |
| PR004533 | 11.9 | 11,308 |
| PR004536 | 52.7 | 15,799 |
| PR004540 | NA | NA |
| PR004949 | 12.8 | 18,260 |
| PR004950 | 14.6 | 13,574 |
| PR004952 | 16.4 | 12,179 |
| PR004953 | NA | NA |
| PR004549 | NA | NA |

TABLE 12
The binding affinity of anti-CLDN18.2
antibodies for HEK293/hCLDN18.2 cells

| | Titer | Efficacy |
|---|---|---|
| | EC₅₀ | Eₘₐₓ |
| Antibody | (nM) | (MFI) |
| PR000400 | 5.60 | 1,319,493 |
| PR007242 | 11.3 | 1,478,230 |
| PR007243 | 13.3 | 1,467,203 |
| PR007244 | 11.7 | 1,301,214 |
| PR007245 | 13.0 | 1,307,122 |
| PR007246 | 14.4 | 1,374,904 |
| PR007247 | 35.3 | 1,272,613 |
| PR007248 | 12.8 | 1,313,402 |

### Example 6: Competitive Binding Activity of Anti-CLDN18.2 HCAb Antibodies

This example is to study the binding of anti-human CLDN18.2 HCAb monoclonal antibodies to the epitope region of human CLDN18.2 antigen. Competitive binding experiments were performed at the cellular level using HEK293/hCLDN18.2 cells overexpressing human CLDN18.2. Briefly, the anti-human CLDN18.2 antibodies PR000400 and PR004549 were biotinylated using a biotinylation kit (ThermoFisher, A35358) according to the instructions. 50 μL of the biotinylated anti-human CLDN18.2 antibodies PR000400 or PR004549 were each mixed well with 50 μL of a corresponding serially diluted non-biotinylated anti-human CLDN18.2 antibody in a 96-well V-bottom plate (Corning, 3894). Then suspension of HEK293/hCLDN18.2 cells overexpressing human CLDN18.2 were adjusted to 3×10⁶ cells/mL, seeded 50 L/well. The cells were co-incubated at 4° C. for 2 h. The cells in each well were washed twice with 200 μL of pre-cooled FACS buffer (2% FBS in PBS) and centrifuged at 500 g at 4° C. for 5 min, and then the supernatant was discarded. After washed twice, a fluorescent secondary antibody (BD, 554060, final concentration of 1 μg/mL) was added and incubated in the dark at 4° C. for 1 h. The cells in each well were washed twice with 200 μL of pre-cooled FACS buffer (2% FBS in PBS) and centrifuged at 500 g at 4° C. for 5 min, and then the supernatant was discarded. Finally, the cells in each well were resuspended in 200 μL of pre-cooled FACS buffer, and the fluorescence signal values were read using a BD FACS CANTOII. The inhibition rate was calculated using the formula, inhibition rate (%)=(A−B)/A×100 (note: A: fluorescence signal after interaction of biotinylated antibody with ISO hIgG1 (Crownbio, C0001-4); B: fluorescence signal after interaction of biotinylated antibody with non-biotinylated antibody).

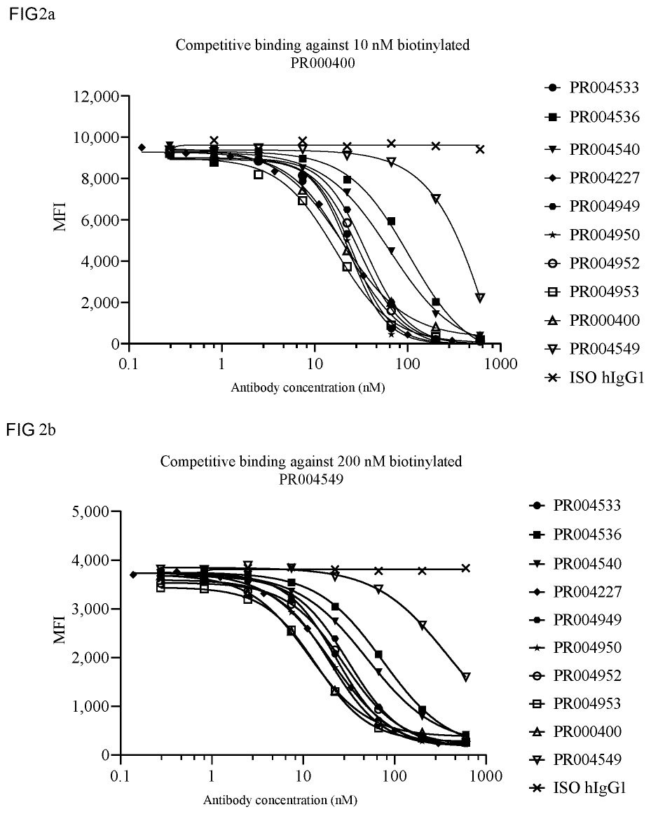

As shown in FIGS. 2 (a-b) and Table 13, the anti-CLDN18.2 HCAb antibodies of the present invention are all able to block the binding of PR000400 or PR004549 to human CLDN18.2, and the detected blocking ability of the antibodies increases with the antibody concentration in a positively correlated manner; the inhibition rate can reach >80%, indicating that the test HCAb has very similar epitopes to those of PR000400 and PR004549. The test antibodies have low binding affinity for HEK293/hCLDN18.1 cells. From the above results, it can be inferred that the test antibodies bind to human CLDN18.2 protein at ECL1 (Extracellular loop 1) rather than ECL2.

TABLE 13
The competitive binding ability of anti-CLDN18.2
antibodies against PR000400 and PR004549

| | 10 nM biotinylated | | 200 nM biotinylated | |
|---|---|---|---|---|
| | PR000400 | | PR004549 | |
| | | Maximum | | Maximum |
| | | inhibition | | inhibition |
| | IC50 | rate | IC50 | rate |
| Antibody | (nM) | (%) | (nM) | (%) |
| PR004533 | 25.5 | 98.6 | 23.2 | 93.7 |
| PR004536 | 103 | 98.0 | 74.1 | 90.1 |
| PR004540 | 64.1 | 95.8 | 50.0 | 89.2 |
| PR004227 | 22.3 | 98.3 | 20.3 | 93.8 |
| PR004949 | 35.4 | 98.5 | 31.0 | 94.2 |
| PR004950 | 23.5 | 98.5 | 19.9 | 94.3 |
| PR004952 | 30.1 | 98.5 | 27.8 | 93.3 |
| PR004953 | 16.7 | 98.0 | 13.8 | 93.2 |
| PR000400 | 20.1 | 96.4 | 11.5 | 90.7 |
| PR004549 | 946 | 76.5 | 379 | 58.4 |
| Iso hIgG1 | NA | 0.00 | NA | 0.00 |

### Example 7: Endocytic Activity of Anti-CLDN18.2 HCAB Antibodies

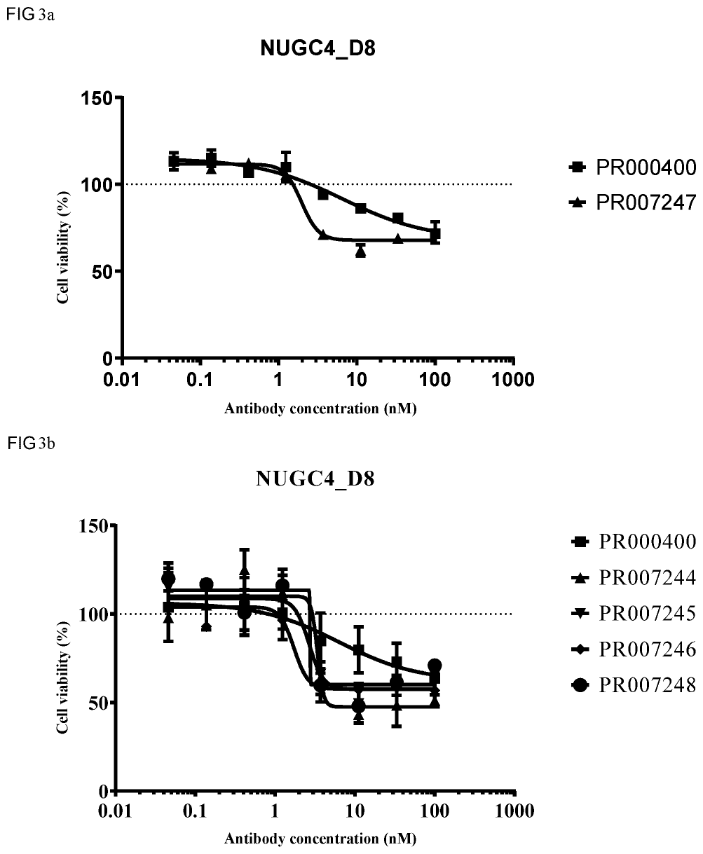

Antibodies were assayed for ability to induce cytotoxic killing against NUGC4_D8 cells when co-cultured with an MMAF-conjugated anti-human IgG antibody (Moradec, Cat #: AH-102-AF) using a CellTiter-Glo luminescent cell viability assay kit (Promega, G7573). NUGC4_D8 cells were centrifuged at 300 g for 5 min and then resuspended in culture medium (RPMI1640+10% FBS) to adjust the density of the cells to 2×10⁴ cells/mL. 50 μL of the cell suspension was added to each well of a 96-well plate. The cells were incubated at 37° C. overnight. Antibodies were diluted to different concentrations with medium, and 25 μL of the antibody dilution was added to each well of the 96-well plate. The MMAF-conjugated anti-human IgG antibody was diluted with medium, and 25 μL of the antibody dilution was added to each well of a 96-well plate to a final concentration of 6.6 nM. The cells were incubated with the antibody at 37° C. for 3 days. The 96-well plate was left standing at room temperature for 30 min, and 100 μL of CellTiter-Glo chromogenic solution was added to each well at room temperature. Then, the sample was incubated in the dark at room temperature for 10 min. The plate was read with PE Enspire. Cell viability (%)=[(luminescent sample)/(luminescent mock control)]×100. PR000400 (IMAB362 analogue) was used as a positive control and Iso hIgG1 (CrownBio, C0001-4) antibody as a negative control. FIGS. 3 (a-b) show the viability of target cells. When co-cultured with the MMAF-conjugated anti-human IgG antibody, the test antibodies exhibit a better cytotoxic effect against NUGC4_D8 cells in a dose-dependent manner, compared to PR000400.

### Example 8: Structure and Design of CLDN18.2×CD3 Bispecific Antibodies

Selected anti-CLDN8.2 and anti-CD3 antibodies were used to prepare a bispecific antibody. The prepared CLDN18.2×CD3 bispecific antibody can bind to two targets simultaneously, with one terminus capable of recognizing CLDN18.2 specifically expressed on tumor cell surfaces and the other terminus capable of binding to CD3 molecules on T cells. After binding to the surface of tumor cells, the CLDN18.2×CD3 bispecific antibody molecule can recruit and activate T cells around the tumor cells, thereby kill the tumor cells.

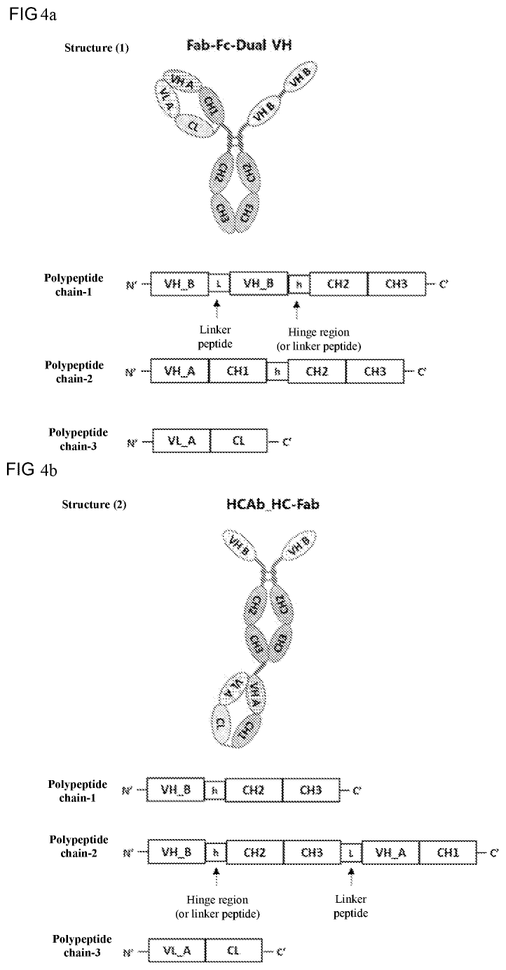

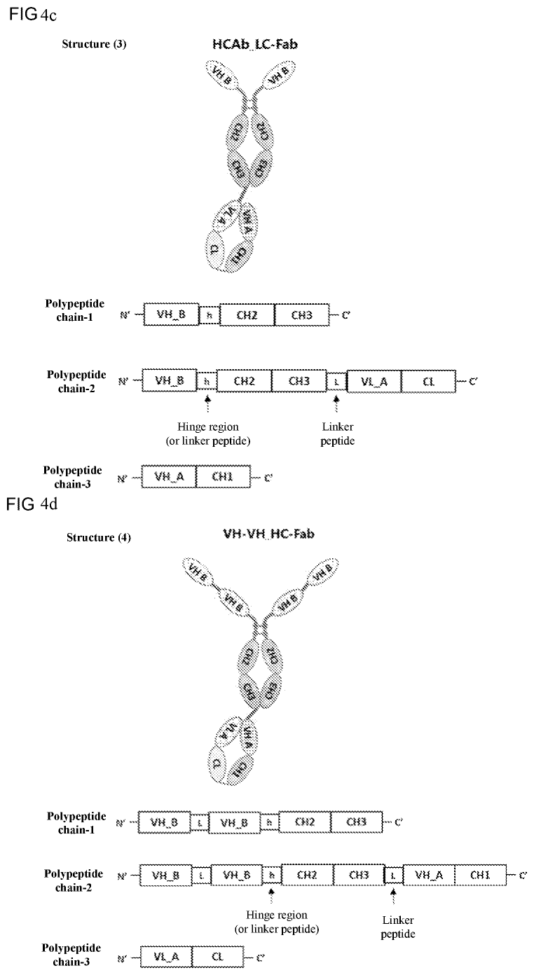

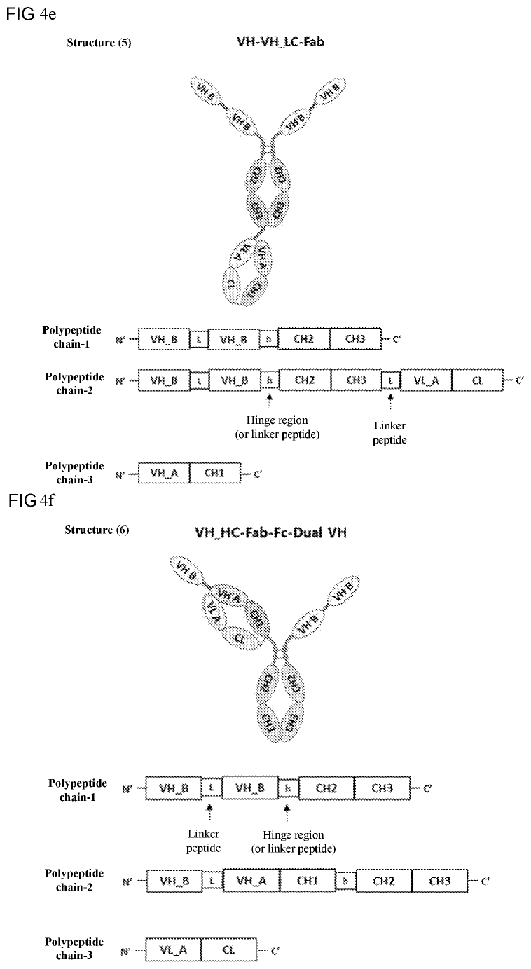

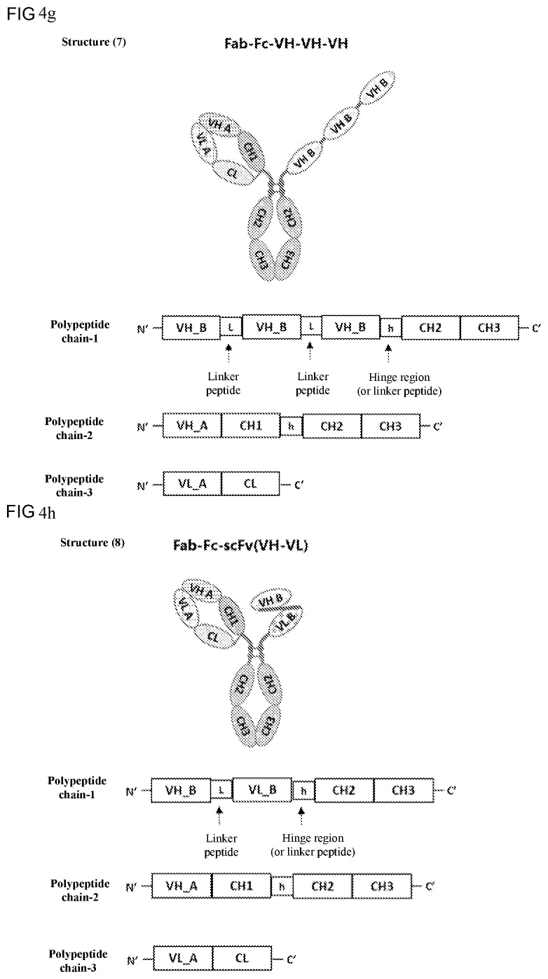

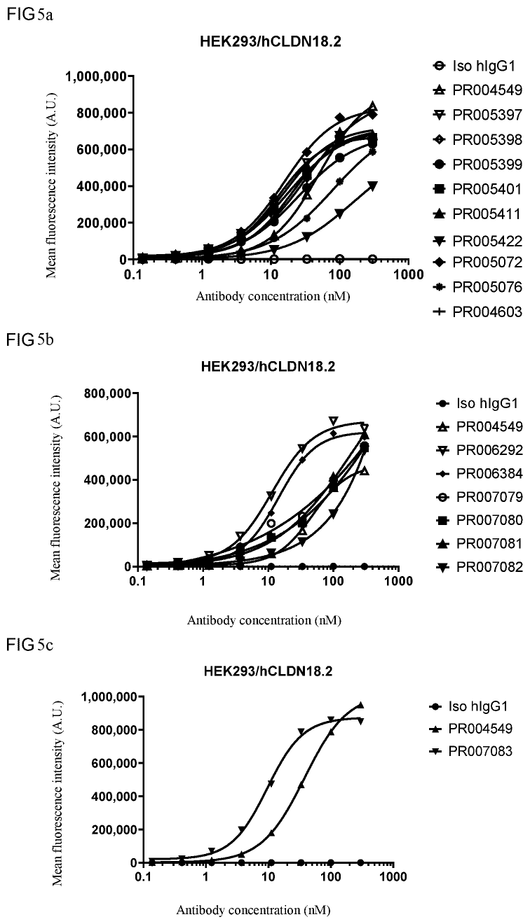

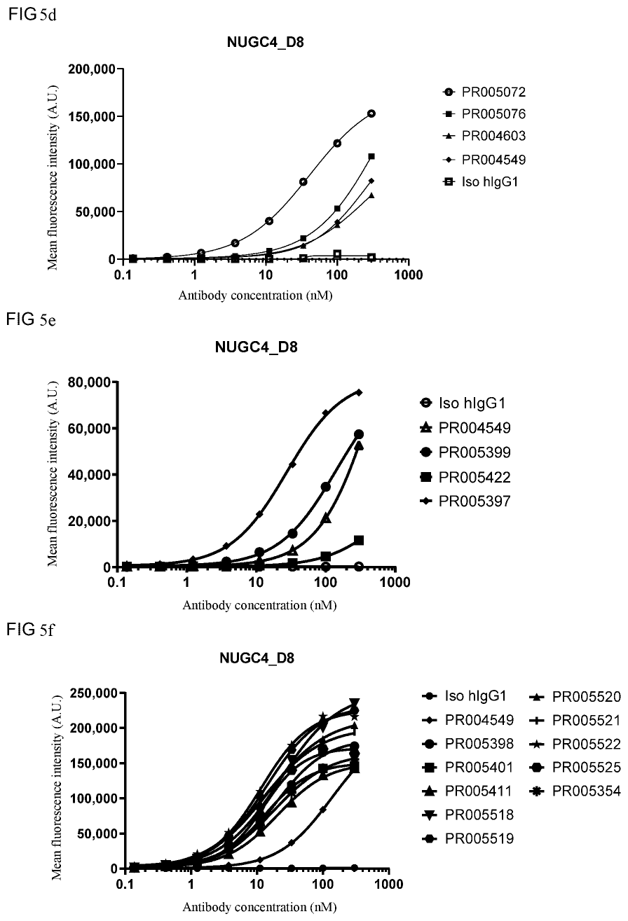

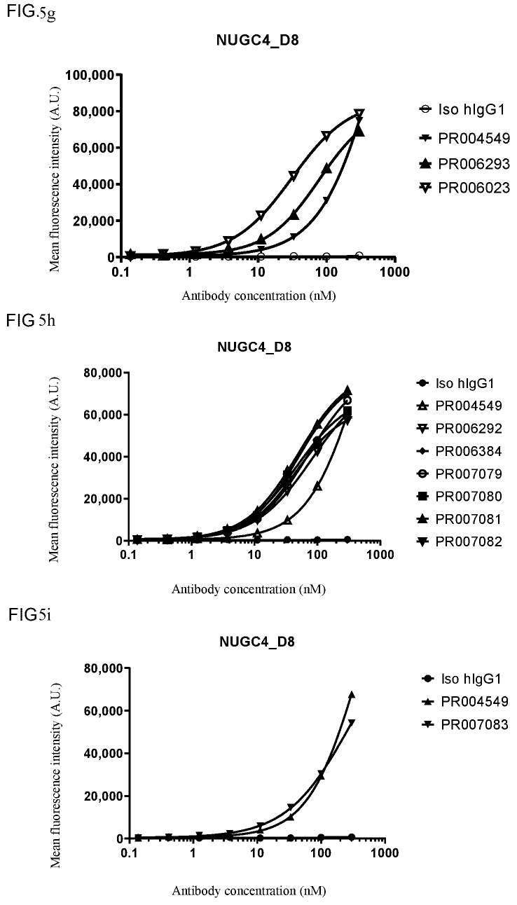

As shown in FIG. 4, the structure (1) is a molecule with “2+1” Fab-Fc-Dual VH asymmetric structure; for the molecule with “2+1” asymmetric structure, the structure involves three protein chains comprising the heavy and light chains of the corresponding anti-CD3 antibody and the Dual VH polypeptide chain of the anti-CLDN18.2 antibody, respectively; the structures (2) and (3) were molecules with “2+1” HCAb-Fc-Fab asymmetric structure, which involves three protein chains comprising the heavy and light chains of the corresponding anti-CD3 antibody and the HCAb polypeptide chain of the anti-CLDN18.2 antibody, respectively.

The structure (4) is a molecule with “4+1” VH-VH_HC-Fab asymmetric structure, which involves three protein chains comprising the heavy and light chains of the corresponding anti-CD3 antibody and the tetravalent VH polypeptide chain of the anti-CLDN18.2 antibody, respectively.

The structure (5) is a molecule with “4+1” VH-VH_LC-Fab asymmetric structure, which involves three protein chains comprising the heavy and light chains of the corresponding anti-CD3 antibody and the tetravalent VH polypeptide chain of the anti-CLDN18.2 antibody, respectively.

The structure (6) is a molecule with “3+1” VH_HC-Fab-Fc-Dual VH asymmetric structure, which involves three protein chains comprising the heavy and light chains of an anti-CD3 antibody with the corresponding N-terminus linked to a monovalent VH of the anti-CLDN18.2 antibody and the bivalent VH polypeptide chain of the anti-CLDN18.2 antibody, respectively.

The structure (7) is a molecule with “3+1” Fab-Fc-VH—VH—VH asymmetric structure, which involves three protein chains comprising the heavy and light chains of the corresponding anti-CD3 antibody and the trivalent VH polypeptide chain of the anti-CLDN18.2 antibody, respectively.

The structure (8) is a molecule with “1+1” Fab-Fc-scFv asymmetric structure, which involves two protein chains comprising the heavy and light chains of the corresponding anti-CD3 antibody and the scFv polypeptide chain of the anti-Lysozyme or anti-CLDN18.1 antibody, respectively.

To minimize the formation of byproducts with mismatched heavy chains (e.g., mismatching of two heavy chains of the anti-CD3 antibody), a mutant heterodimeric Fc region was used. It carries a “knob-hole” mutation and a modified disulfide bond, as described in WO2009080251 and WO2009080252. The CLDN18.2×CD3 bispecific antibody has an Fc of IgG1 and carries L234A, L235A or L234A, L235A and G237A (numbered according to the EU index) mutations on CH3 of the Fc. Each bispecific antibody was generated by co-transfecting simultaneously three or four different mammalian expression vectors encoding: 1) the heavy chain of the corresponding anti-CLDN18.2 antibody, which carries a “Hole” mutation in the Fc region so as to produce a heterodimeric antibody, CH3 of the Fc carrying L234A, L235A or L234A, L235A and G237A mutations; 2) the heavy chain of the corresponding anti-CD3 antibody, which carries a “knob” mutation in the Fc region so as to produce a heterodimeric antibody, CH3 of the Fc carrying L234A, L235A or L234A, L235A and G237A mutations; and 3) the light chain of the corresponding anti-CD3 antibody. The “knob” mutation in the Fc region of human IgG1 consists of: T366W, and the “Hole” mutation consists of: T366S, L368A, and Y407V. In addition, S354C in the “knob” Fc region and “Hole” Y349C may be included; they form a pair of disulfide bonds to increase the stability and the yield of the heterodimeric antibody.

Specific information about the CLDN18.2×CD3 bispecific antibodies constructed in the present invention is shown in Tables 14, 15 and 16.

TABLE 14
The CLDN18.2 × CD3 bispecific antibodies constructed according to the structures (1)-(8) of this example

| | | | | | | | | Fc type of |
|---|---|---|---|---|---|---|---|---|
| | Bispecific | | | CD3- | CLDN18.2- | | | CLDN18.2 |
| | antibodies | | CLDN18.2 | terminus | terminus | Linker | Fc type of CD3 end | terminus |
| Structure | molecule | CD3 antibody | antibody | structure | structure | peptide | (mutation) | (mutation) |
| 1 | PR004603 | PR003886 | PR004227 | Fab | Dual VH | GS_15 | Human IgG1 (knob, | Human IgG1 (hole, |
| | | | | | | | LALA) | LALA) |
| 1 | PR005072 | PR003886 | PR004533 | Fab | Dual VH | GS_15 | Human IgG1 (knob, | Human IgG1 (hole, |
| | | | | | | | LALA) | LALA) |
| 1 | PR005076 | PR003886 | PR004536 | Fab | Dual VH | GS_15 | Human IgG1 (knob, | Human IgG1 (hole, |
| | | | | | | | LALA) | LALA) |
| 1 | PR005354 | PR001848 | PR004533 | Fab | Dual VH | GS_15 | Human IgG1 (knob, | Human IgG1 (hole, |
| | | | | | | | LALA) | LALA) |
| 1 | PR005397 | PR003886 | PR007246 | Fab | Dual VH | GS_15 | Human IgG1 (knob, | Human IgG1 (hole, |
| | | | | | | | LALA) | LALA) |
| 1 | PR005398 | PR003886 | PR007242 | Fab | Dual VH | GS_15 | Human IgG1 (knob, | Human IgG1 (hole, |
| | | | | | | | LALA) | LALA) |
| 1 | PR005399 | PR003886 | PR007243 | Fab | Dual VH | GS_15 | Human IgG1 (knob, | Human IgG1 (hole, |
| | | | | | | | LALA) | LALA) |
| 1 | PR005401 | PR003886 | PR007244 | Fab | Dual VH | GS_15 | Human IgG1 (knob, | Human IgG1 (hole, |
| | | | | | | | LALA) | LALA) |
| 1 | PR005411 | PR003886 | PR007245 | Fab | Dual VH | GS_15 | Human IgG1 (knob, | Human IgG1 (hole, |
| | | | | | | | LALA) | LALA) |
| 1 | PR005422 | PR003886 | PR007247 | Fab | Dual VH | GS_15 | Human IgG1 (knob, | Human IgG1 (hole, |
| | | | | | | | LALA) | LALA) |
| 1 | PR006023 | PR001848 | PR007246 | Fab | Dual VH | GS_15 | Human IgG1 (knob, | Human IgG1 (hole, |
| | | | | | | | LALA) | LALA) |
| 1 | PR006292 | PR003886 | PR007245 | Fab | Dual VH | GS_15 | Human IgG1 (knob, | Human IgG1 (hole, |
| | | | | | | | AAA) | AAA) |
| 1 | PR006293 | PR001848 | PR007245 | Fab | Dual VH | GS_15 | Human IgG1 (knob, | Human IgG1 (hole, |
| | | | | | | | AAA) | AAA) |
| 1 | PR006384 | PR003886 | PR007246 | Fab | Dual VH | GS_15 | Human IgG1 (knob, | Human IgG1 (hole, |
| | | | | | | | AAA) | AAA) |
| 1 | PR007079 | PR003886 | PR007243 | Fab | Dual VH | GS_15 | Human IgG1 (knob, | Human IgG1 (hole, |
| | | | | | | | AAA) | AAA) |
| 1 | PR007080 | PR003886 | PR007244 | Fab | Dual VH | GS_15 | Human IgG1 (knob, | Human IgG1 (hole, |
| | | | | | | | AAA) | AAA) |
| 1 | PR007081 | PR003886 | PR007242 | Fab | Dual VH | GS_15 | Human IgG1 (knob, | Human IgG1 (hole, |
| | | | | | | | AAA) | AAA) |
| 1 | PR007082 | PR003886 | PR007248 | Fab | Dual VH | GS_15 | Human IgG1 (knob, | Human IgG1 (hole, |
| | | | | | | | AAA) | AAA) |
| 1 | PR007083 | PR003886 | PR007245 | Fab | Dual VH | GS_15 | Human IgG1 (knob, | Human IgG1 (hole, |
| | | | | | | | AAA) | AAA) |
| 2 | PR005518 | PR001848 | PR004533 | Fab | VH | GS_15 | Human IgG1 (knob, | Human IgG1 (hole, |
| | | | | | | | LALA) | LALA) |
| 3 | PR005519 | PR001848 | PR004533 | Fab | VH | GS_15 | Human IgG1 (knob, | Human IgG1 (hole, |
| | | | | | | | LALA) | LALA) |
| 4 | PR005520 | PR001848 | PR004533 | Fab | Dual VH | GS_15 | Human IgG1 (knob, | Human IgG1 (hole, |
| | | | | | | | LALA) | LALA) |
| 5 | PR005521 | PR001848 | PR004533 | Fab | Dual VH | GS_15 | Human IgG1 (knob, | Human IgG1 (hole, |
| | | | | | | | LALA) | LALA) |
| 6 | PR005522 | PR001848 | PR004533 | Fab | Trivalent VH | GS_15 | Human IgG1 (knob, | Human IgG1 (hole, |
| | | | | | | | LALA) | LALA) |
| 7 | PR005525 | PR003886 | PR004533 | Fab | Trivalent VH | GS_15 | Human IgG1 (knob, | Human IgG1 (hole, |
| | | | | | | | LALA) | LALA) |
| 8 | PR004312 | PR001848 | PR000325 | Fab | scFv | GS_20 | Human IgG1 (knob, | Human IgG1 (hole, |
| | | | | | | | LALA) | LALA) |
| 8 | PR004313 | PR001848 | PR001861 | Fab | scFv | GS_20 | Human IgG1 (knob, | Human IgG1 (hole, |
| | | | | | | | LALA) | LALA) |

TABLE 15
The sequence numbers of the CLDN18.2 × CD3
bispecific antibody of the present invention

| | SEQ ID NOs: | | |
|---|---|---|---|
| | Polypeptide | Polypeptide | Polypeptide |
| Antibody | chain-1 | chain-2 | chain-3 |
| PR004603 | 214 | 213 | 200 |
| PR005072 | 219 | 213 | 200 |
| PR005076 | 220 | 213 | 200 |
| PR005397 | 221 | 213 | 200 |
| PR005398 | 222 | 213 | 200 |
| PR005399 | 223 | 213 | 200 |
| PR005401 | 224 | 213 | 200 |
| PR005411 | 225 | 213 | 200 |
| PR005422 | 226 | 213 | 200 |
| PR005518 | 227 | 228 | 200 |
| PR005519 | 227 | 230 | 229 |
| PR005520 | 219 | 231 | 200 |
| PR005521 | 219 | 232 | 229 |
| PR005522 | 219 | 233 | 200 |
| PR005525 | 234 | 213 | 200 |
| PR005354 | 219 | 209 | 200 |
| PR006023 | 221 | 209 | 200 |
| PR006292 | 236 | 235 | 200 |
| PR006293 | 236 | 237 | 200 |
| PR006384 | 238 | 235 | 200 |
| PR007079 | 239 | 235 | 200 |
| PR007080 | 240 | 235 | 200 |
| PR007081 | 241 | 235 | 200 |
| PR007082 | 242 | 235 | 200 |
| PR007083 | 243 | 235 | 200 |

TABLE 16
The sequence numbers of the CDRs of the antigen-binding domain of
the CLDN18.2 × CD3 bispecific antibody of the present invention

| | | Antigen- | | | | | | |
|---|---|---|---|---|---|---|---|---|
| | | binding | | | | | | |
| Structure | Molecule | domain | SEQ ID NOs: | | | | | |
| number | number | number | LCDR1 | LCDR2 | LCDR3 | HCDR1 | HCDR2 | HCDR3 |
| 1 | PR004603 | #1 | 101 | 116 | 131 | 11 | 38 | 72 |
| | | #2 | | | | 16 | 42 | 77 |
| 1 | PR005072 | #1 | 101 | 116 | 131 | 11 | 38 | 72 |
| | | #2 | | | | 16 | 43 | 78 |
| 1 | PR005076 | #1 | 101 | 116 | 131 | 11 | 38 | 72 |
| | | #2 | | | | 16 | 44 | 79 |
| 1 | PR005354 | #1 | 101 | 116 | 131 | 11 | 38 | 72 |
| | | #2 | | | | 16 | 43 | 78 |
| 1 | PR005397, | #1 | 101 | 116 | 131 | 11 | 38 | 72 |
| | PR006384 | #2 | | | | 16 | 52 | 78 |
| 1 | PR005398, | #1 | 101 | 116 | 131 | 11 | 38 | 72 |
| | PR007081 | #2 | | | | 16 | 48 | 78 |
| 1 | PR005399, | #1 | 101 | 116 | 131 | 11 | 38 | 72 |
| | PR007079 | #2 | | | | 16 | 49 | 78 |
| 1 | PR005401, | #1 | 101 | 116 | 131 | 11 | 38 | 72 |
| | PR007080 | #2 | | | | 16 | 50 | 78 |
| 1 | PR005411, | #1 | 101 | 116 | 131 | 11 | 38 | 72 |
| | PR006292, | #2 | | | | 16 | 51 | 78 |
| | PR007083 | | | | | | | |
| 1 | PR005422 | #1 | 101 | 116 | 131 | 11 | 38 | 72 |
| | | #2 | | | | 16 | 53 | 79 |
| 1 | PR006023 | #1 | 101 | 116 | 131 | 11 | 38 | 72 |
| | | #2 | | | | 16 | 52 | 78 |
| 1 | PR006293 | #1 | 101 | 116 | 131 | 11 | 38 | 72 |
| | | #2 | | | | 16 | 51 | 78 |
| 1 | PR007082 | #1 | 101 | 116 | 131 | 11 | 38 | 72 |
| | | #2 | | | | 16 | 54 | 78 |
| 2 | PR005518 | #1 | 101 | 116 | 131 | 11 | 38 | 72 |
| | | #2 | | | | 16 | 43 | 78 |
| 3 | PR005519 | #1 | 101 | 116 | 131 | 11 | 38 | 72 |
| | | #2 | | | | 16 | 43 | 78 |
| 4 | PR005520 | #1 | 101 | 116 | 131 | 11 | 38 | 72 |
| | | #2 | | | | 16 | 43 | 78 |
| 5 | PR005521 | #1 | 101 | 116 | 131 | 11 | 38 | 72 |
| | | #2 | | | | 16 | 43 | 78 |
| 6 | PR005522 | #1 | 101 | 116 | 131 | 11 | 38 | 72 |
| | | #2 | | | | 16 | 43 | 78 |
| 7 | PR005525 | #1 | 101 | 116 | 131 | 11 | 38 | 72 |
| | | #2 | | | | 16 | 43 | 78 |

### Example 9: Preparation and Characterization of CLDN18.2×CD3 Bispecific Antibodies

### 9.1. Preparation of Recombinant CLDN18.2×CD3 Bispecific Antibodies

A plurality of plasmids encoding recombinant CLDN18.2×CD3 bispecific antibodies were transfected into mammalian host cells (e.g., Chinese Hamster Ovary (CHO) cells) according to a certain proportion, and the corresponding purified recombinant antibody can be obtained using conventional recombinant protein expression and purification techniques. Specifically, ExpiCHO-S™ cells (Gibco, A29127) were expanded in ExpiCHO™ Expression Medium (Gibco, A2910001). Before the transient transfection, the cells were adjusted to a concentration of 3× 10⁶ to 4×10⁶ cells/mL, and cultured in an 8% CO₂ shaker at 37° C. for 24 h, leading to a cell concentration of 7×10⁶ to 10×10⁶ cells/mL. The cells were then diluted to 6× 10⁶ cells/mL, and 10 mL of the cultured cells was prepared. A total of 8 μg of the above plasmids encoding CLDN18.2×CD3 bispecific antibodies (the ratio of the plasmids to cells is 0.8 μg:1 mL) was dissolved in 0.4 mL of OptiPRO™ SFM medium (Gibco, 12309019). The resulting mixture was filtered through a 0.22 μm filter for sterilization. Then 32 μL of ExpiFectamine™ CHO reagent (Gibco, A29129) was added to 0.37 mL of OptiPRO™ SFM medium (Gibco, 12309019). The ExpiFectamine™ CHO reagent solution was immediately added slowly to the plasmid solution. The mixture was inverted to be well mixed. The mixed solution of plasmid and transfection reagent was slowly added dropwise while shaking the flask. The cells were cultured in an 8% CO₂ shaker at 37° C. for 8-9 days. The Cell viability was observed after 8 days.

The culture was collected and centrifuged at 3300 g for 10 min, and then the supernatant was collected and centrifuged at high speed to remove impurities. A gravity column (Bio-Rad, 7311550) containing MabSelect™ (GE Healthcare Life Science, 71-5020-91 AE) was equilibrated with PBS (pH 7.4) and rinsed with 2-5 column volumes of PBS. The supernatant sample was loaded onto a column. The column was rinsed with 5-10 column volumes of PBS. The target protein was eluted with 0.1 M glycine (pH 3.5). The eluate was adjusted to neutrality with Tris-HCl (pH 8.0), and concentrated and buffer exchanged into PBS buffer with an ultrafiltration tube (Millipore, UFC901024) to obtain a purified antibody solution. Then, the purified antibody solution was subjected to concentration determination using NanoDrop (Thermo Scientific™ NanoDrop™ One), subpackaged and stored for later use.

### 9.2. Antibody Characterization by SEC-HPLC, HIC-HPLC and DSF

A proper amount of the purified sample above was loaded onto an analytical SEC column TSKgel G3000SWxl (HPLC system model: Agilent 1260 Infinity II) for the measurement of purity. In this method, the following parameters and conditions were used: mobile phase: 1×PBS, pH 7.4 (Sangon, E607016); room temperature; flow rate: 1.0 mL/min; sample concentration: 1 mg/mL; injection volume: 20 μL; detection wavelength: 280 nm. After being recorded, the chromatogram was integrated using ChemStation software and relevant data were calculated. An analysis was generated, with the retention times reported for different components in the sample.

A proper amount of the purified sample above was loaded onto an analytical HIC column TSKgel Butyl-NPR 4.6*35 (HPLC system model: Agilent 1260 Infinity II) for the measurement of purity and hydrophobicity. The method consisted of a linear gradient from 100% mobile phase A (20 mM PB, 1.8 M (NH4)₂SO₄, pH 6.0) to 100% mobile phase B (20 mM PB, pH 6.0) within 16 min. The flow rate was set at 0.7 mL/min, the sample concentration was 1 mg/mL, the injection volume was 20 μL, and the detection wavelength was 280 nm. After being recorded, the chromatogram was integrated using ChemStation software and relevant data were calculated. An analysis was generated, with the retention times reported for different components in the sample.

In this example, the thermal denaturation temperature (Tm) of a protein molecule was measured by differential scanning fluorimetry (DSF). 10 μg of protein was added to a 96-well PCR plate (Thermo, AB-0700/W), followed by the addition of 2 μL of 100× diluted dye SYPRO™ (Invitrogen, 2008138), and then buffer was added to give a final volume of 40 μL per well. The PCR plate was sealed, placed in a real-time fluorescent quantitative PCR instrument (Bio-Rad CFX96 PCR System), incubated at 25° C. for 5 min, then gradually warmed from 25° C. to 95° C. at a gradient of 0.2° C./0.2 min, and cooled to 25° C. at the end of the test. The FRET scanning mode was used and data analysis was performed using Bio-Rad CFX Maestro software to calculate the Tm of the sample. The results of the above characterization are shown in Table 17 below.

TABLE 17
Characterization of the CLDN18.2 × CD3 bispecific antibodies

| | SEC-HPLC | HIC-HPLC | HIC-HPLC | | |
|---|---|---|---|---|---|
| | purity | purity | retention | Tm1 | Tm2 |
| Antibody | (%) | (%) | time (min) | (° C.) | (° C.) |
| PR004549 | 93.4 | 100.0 | 19.0 | 56.0 | NA |
| PR004603 | 100.0 | 100.0 | 19.2 | 50.0 | 66.8 |
| PR005072 | 99.1 | 100.0 | 16.4 | 59.0 | 64.2 |
| PR005076 | 97.8 | 92.1 | 18.3 | 50.2 | 66.8 |
| PR005397 | 100.0 | 100.0 | 16.2 | 52.0 | 59.0 |
| PR005398 | 98.9 | 100.0 | 16.7 | 54.2 | 67.8 |
| PR005399 | 86.0 | 100.0 | 16.5 | 53.8 | 67.8 |
| PR005401 | 99.6 | 100.0 | 16.1 | 60.0 | 67.4 |
| PR005411 | 100.0 | 100.0 | 16.2 | 60.2 | 67.4 |
| PR005422 | 99.7 | 100.0 | 18.0 | 48.4 | 66.4 |
| PR005518 | 64.0 | 100.0 | 16.0 | 53.4 | 64.4 |
| PR005519 | 68.9 | 100.0 | 15.9 | 53.4 | 64.2 |
| PR005520 | 89.7 | 100.0 | 16.4 | 54.0 | 62.8 |
| PR005521 | 89.0 | 100.0 | 16.3 | 54.8 | 62.6 |
| PR005522 | 100.0 | 100.0 | 16.5 | 52.0 | 63.8 |
| PR005525 | 99.7 | 100.0 | 16.4 | 63.2 | 66.4 |
| PR005354 | 99.8 | | | | |
| PR006023 | 100.0 | 100.0 | 16.0 | 59.2 | 65.8 |
| PR006292 | 99.4 | 100.0 | 15.9 | 59.6 | 66.8 |
| PR006293 | 99.0 | 100.0 | 16.0 | 59.8 | 65.8 |
| PR006384 | 100.0 | 100.0 | 15.9 | 59.2 | 67.0 |
| PR007079 | 99.4 | 100.0 | 16.1 | 55.6 | 66.4 |
| PR007080 | 97.8 | 100.0 | 16.0 | 59.6 | 66.8 |
| PR007081 | 95.8 | 100.0 | 16.2 | 56.2 | 66.6 |
| PR007082 | 98.5 | 86.10 | 16.1 | 60.8 | 66.8 |
| PR007083 | 84.5 | 100.0 | 16.1 | 51.8 | 67.6 |
| Trastuzumab | 100.0 | 100.0 | 16.0 | 67.8 | 79.2 |

### Example 10: Binding Affinity of CLDN18.2×CD3 Bispecific Antibodies to Cells

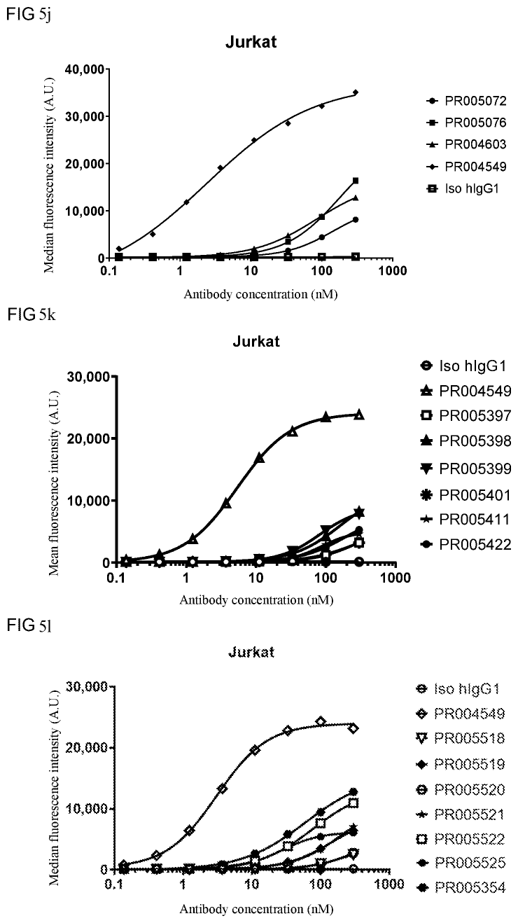

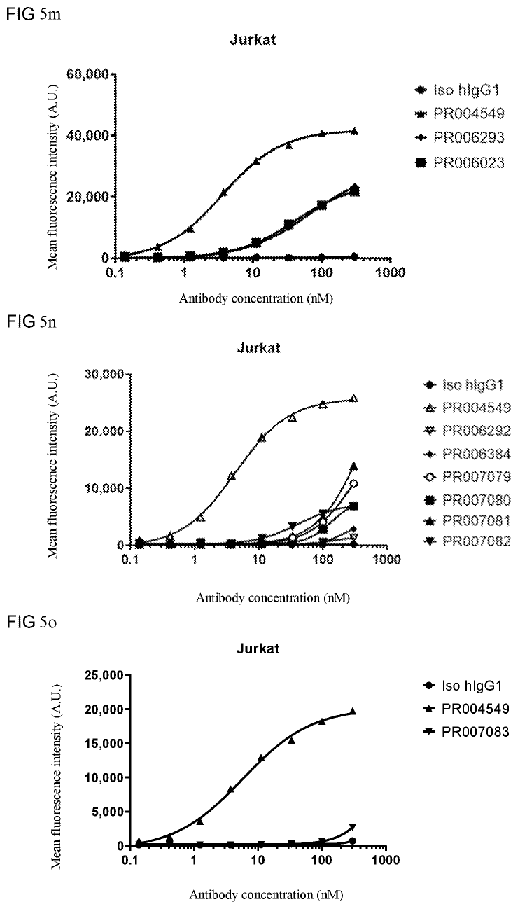

Antibodies were assayed for binding affinity by FACS. Test cells include HEK293/hCLDN18.2, HEK293/hCLDN18.1, NUGC4_D8 and Jurkat cells. The binding affinity was determined as follows: cells were centrifuged at 300 g for 5 min and then resuspended in FACS buffer (PBS containing 2% FBS). The cell density was adjusted to 10⁶ cells/mL, and 50 μL of the cell suspension was added to each well of a 96-well plate. Antibodies were diluted to different concentrations with FACS buffer, and 50 μL of the antibody dilution was added to each well of the 96-well plate. After 2 h of incubation at 4° C., the plate was washed twice with FACS buffer. Then, an FACS buffer containing an APC-conjugated goat anti-human IgG secondary antibody (final concentration of 1.5 μg/mL, Jackson, 109-605-098) was added. After 1 h of incubation at 4° C., the plate was washed twice with FACS buffer. The cells were resuspended in fixative solution, and then the fluorescence of the cells was monitored using an FACS instrument (ACEA NovoCyte). FIG. 5 and Table 18 show the binding affinity of antibodies to the cells. All the test antibodies can bind to CLDN18.2 and CD3-expressing cells, and do not bind to CLDN18.1-expressing cells.

TABLE 18
The binding affinity of CLDN18.2 × CD3
bispecific antibodies for cells

| | Titer EC₅₀ (nM) | | |
|---|---|---|---|
| Antibody | NUGC4_D8 | HEK293/hCLDN18.2 | Jurkat |
| PR004549 | NA | 50.4 | 3.6 |
| PR004603 | NA | 33.1 | NA |
| PR005072 | 90.7 | 15.7 | NA |
| PR005076 | NA | NA | NA |
| PR005397 | 27.5 | 12.8 | NA |
| PR005398 | 19.4 | 20.4 | NA |
| PR005399 | NA | 25.3 | 81.9 |
| PR005401 | 13.1 | 17.5 | NA |
| PR005411 | 20.5 | 16.0 | 85.9 |
| PR005422 | NA | NA | NA |
| PR005518 | 20.5 | | NA |
| PR005519 | 13.9 | | NA |
| PR005520 | 13.9 | | NA |
| PR005521 | 13.9 | | NA |
| PR005522 | 13.9 | | NA |
| PR005525 | 12.6 | | 26.8 |
| PR005354 | 12.6 | | NA |
| PR006023 | 31.3 | | 40.6 |
| PR006292 | 70.1 | 11.5 | NA |
| PR006293 | 78.0 | | 65.0 |
| PR006384 | 51.4 | 14.4 | NA |
| PR007079 | 73.2 | NA | NA |
| PR007080 | 46.7 | NA | NA |
| PR007081 | 49.3 | NA | NA |
| PR007082 | 44.2 | NA | 41.0 |
| PR007083 | NA | 9.6 | NA |

Note:
the blanks in the table indicate that the molecule was not subjected to an affinity assay for HEK293/hCLDN18.2 cells; NA indicates a failure to obtain EC₅₀ by fitting for the molecule in an affinity assay for HEK293/hCLDN18.2 cells.

### Example 11: TDCC Activity of CLDN18.2×CD3 Bispecific Antibodies

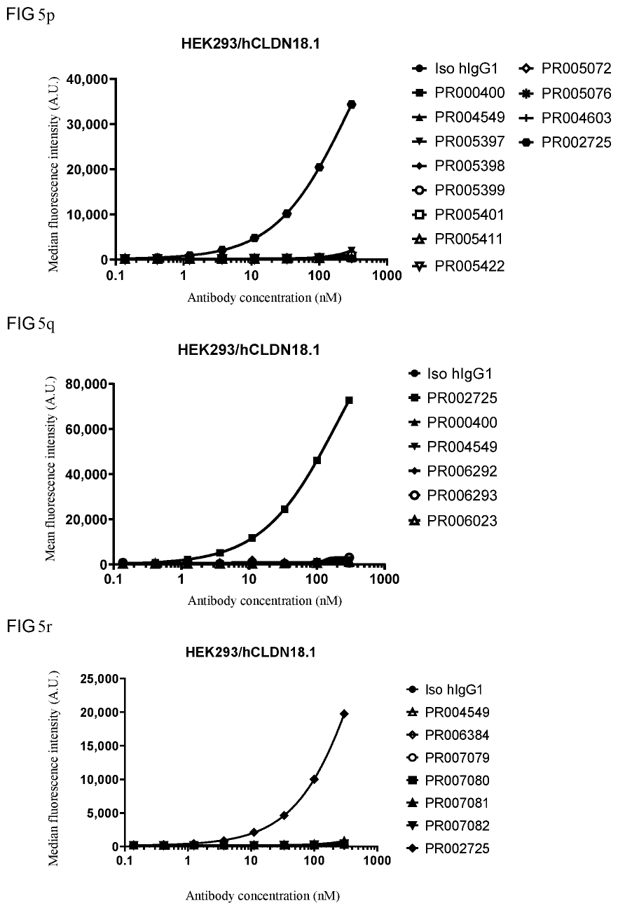

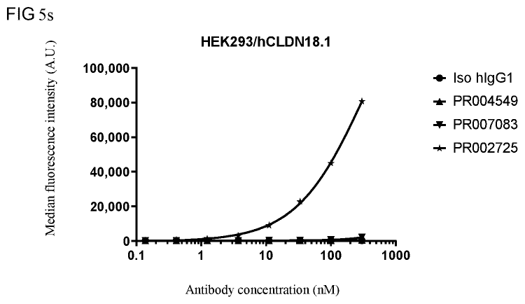

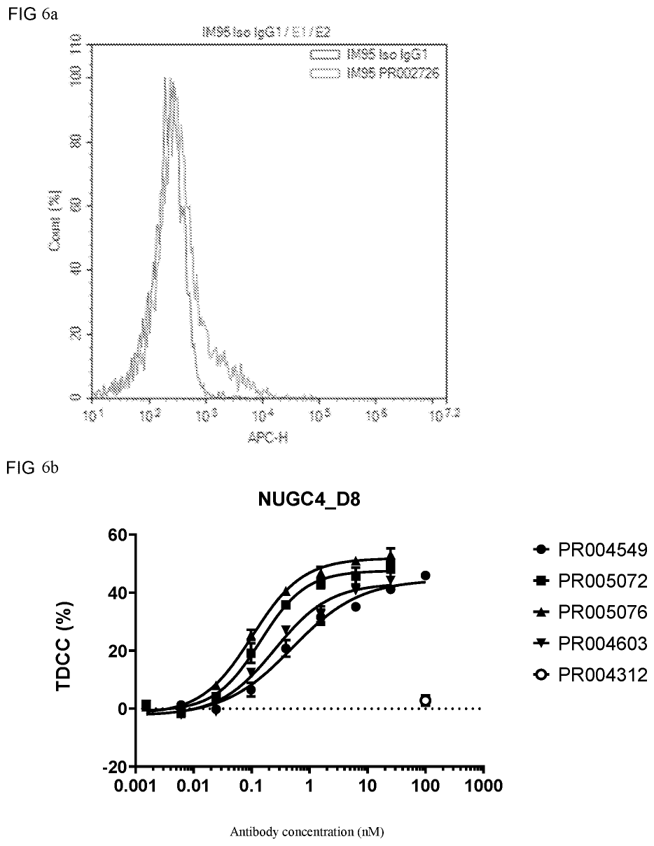

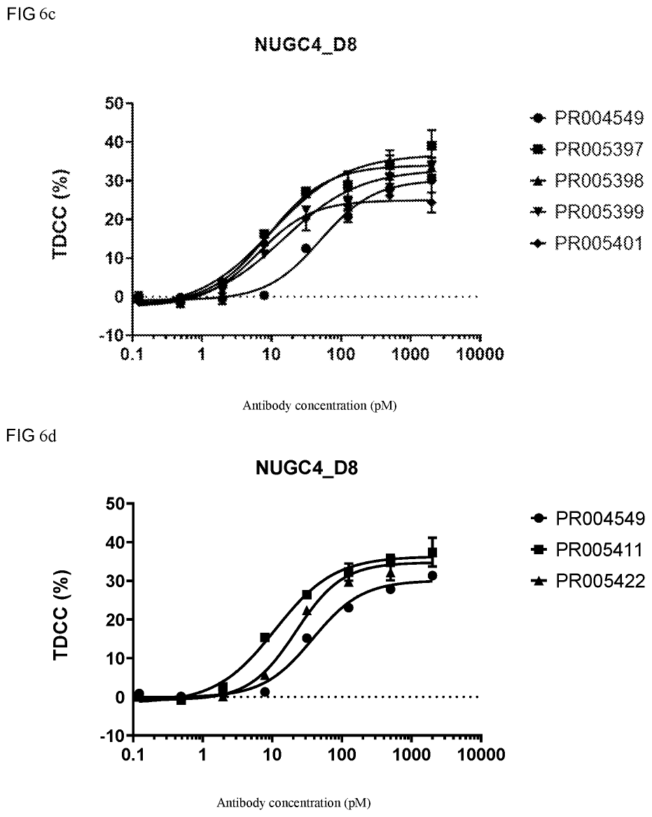

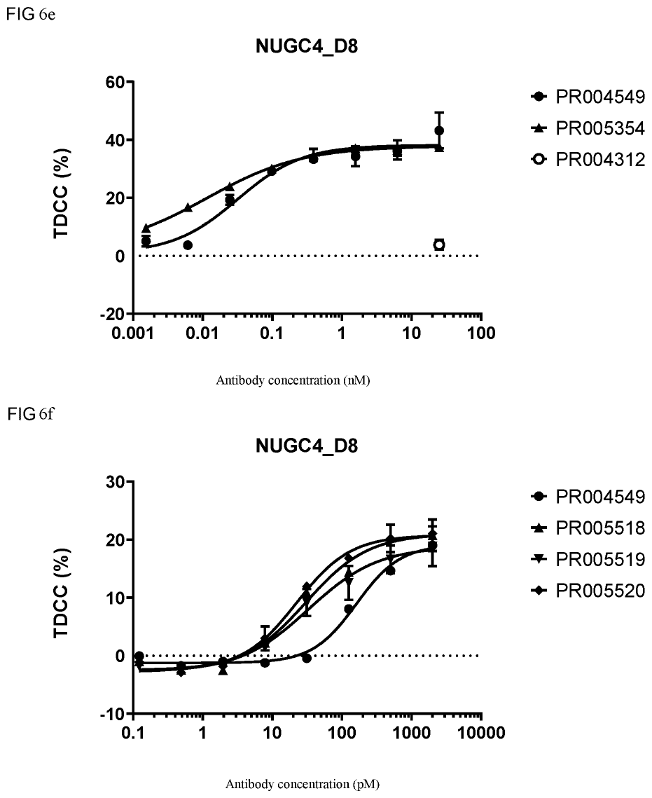

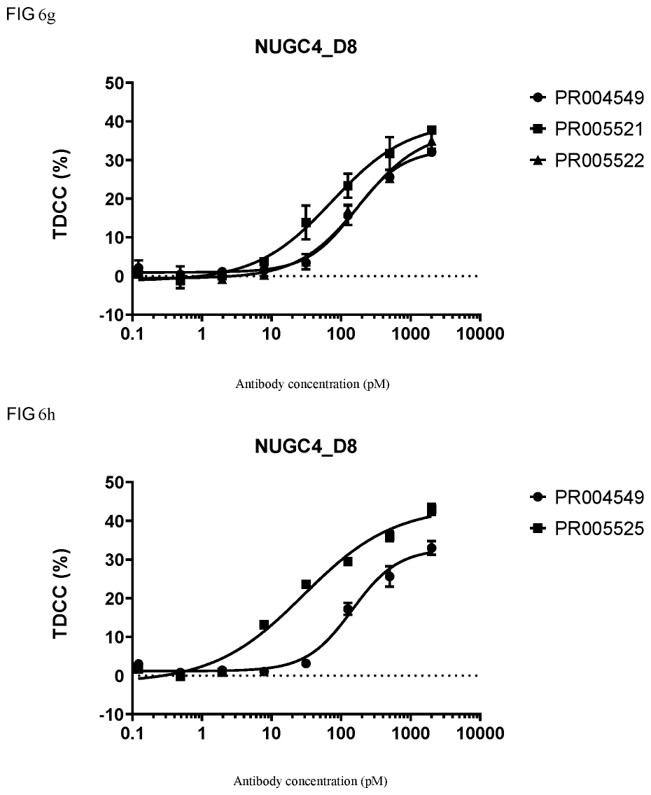

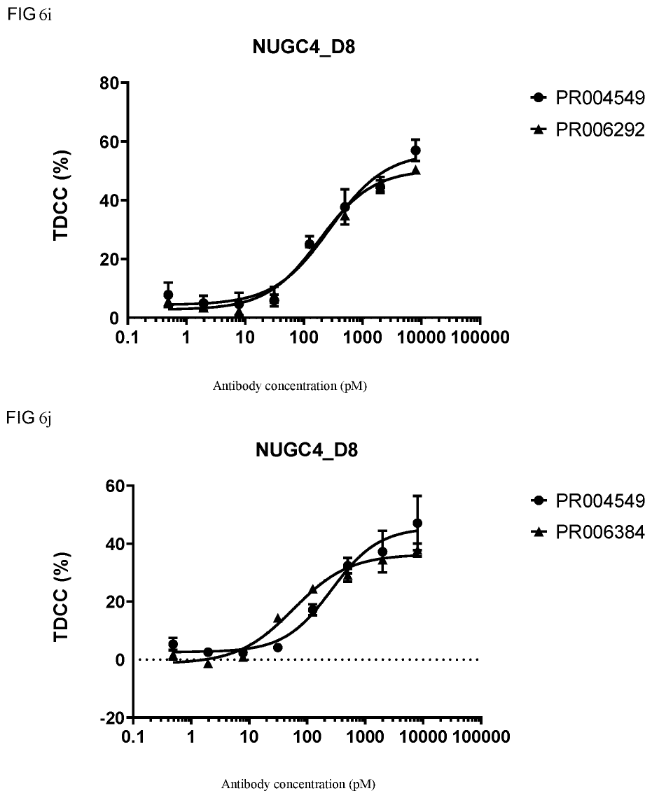

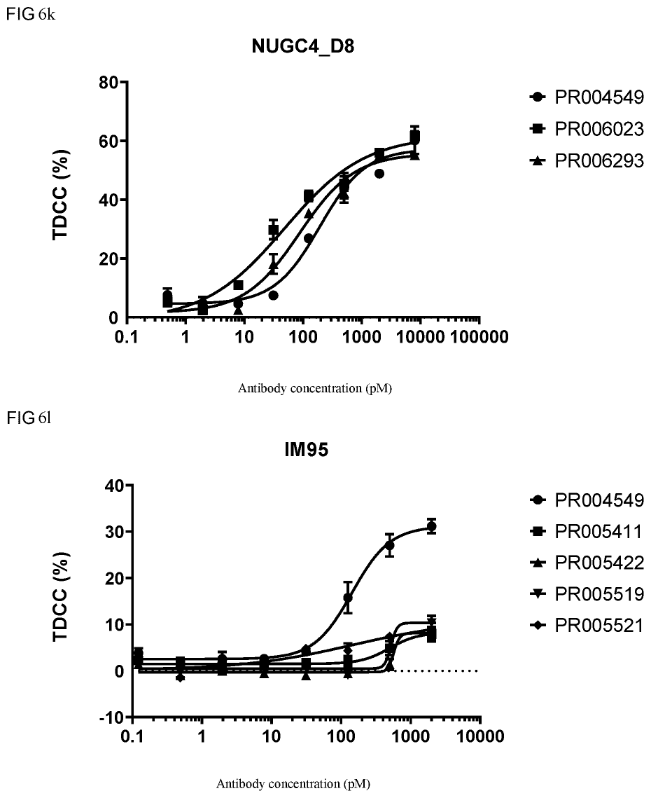

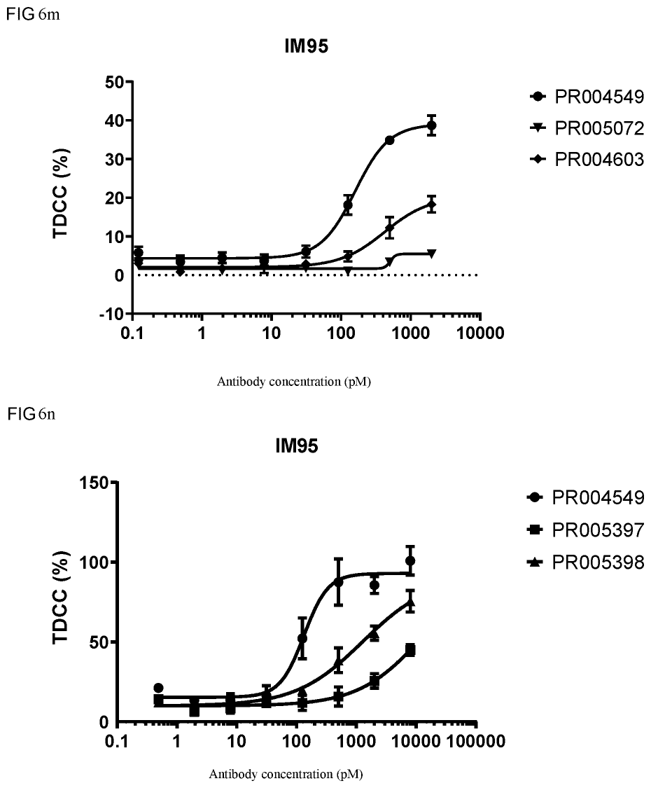

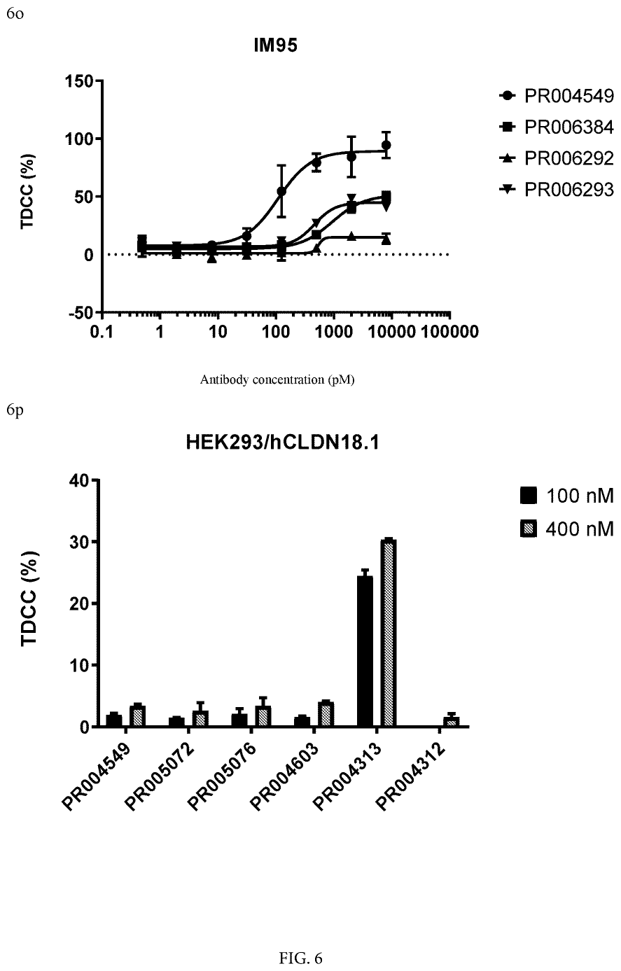

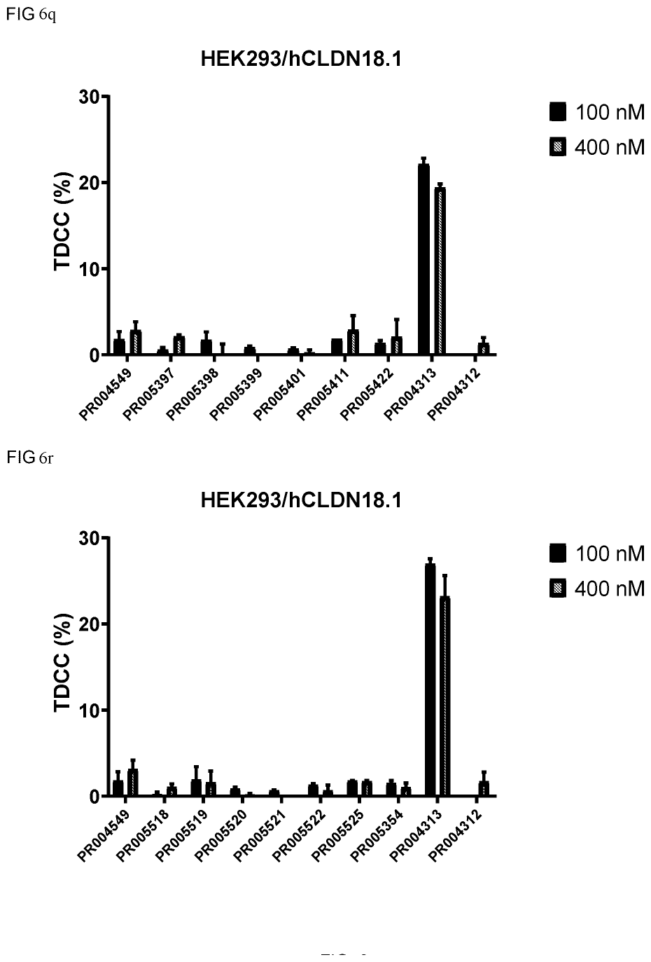

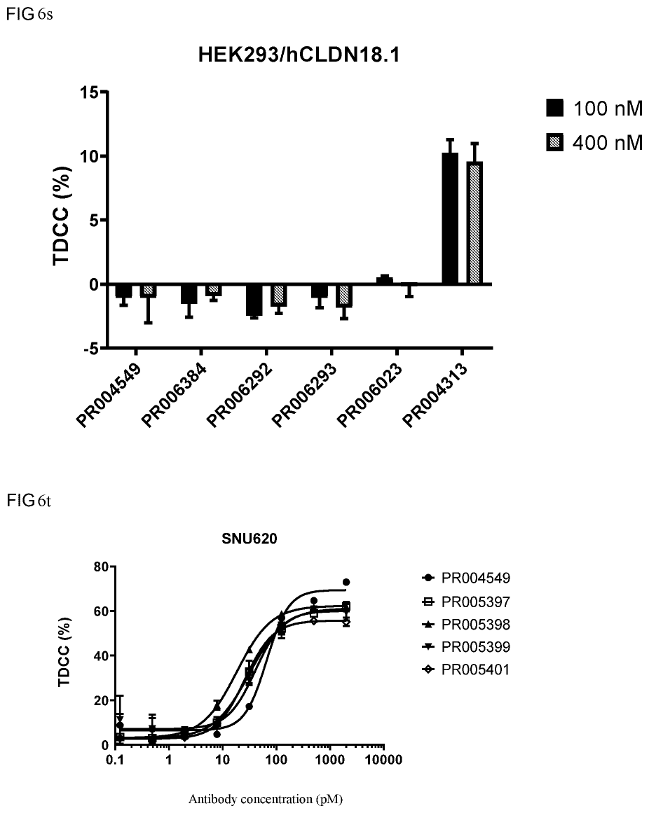

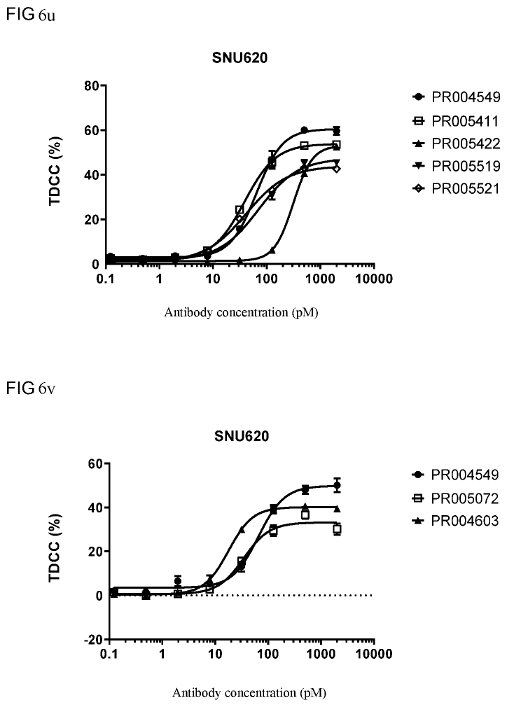

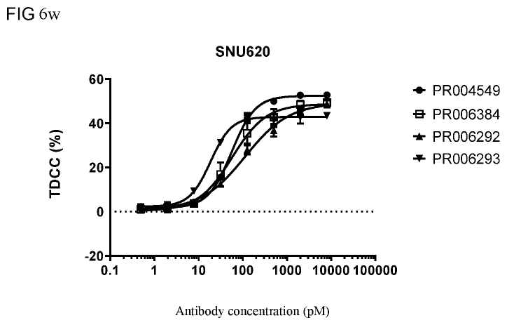

CLDN18.2×CD3 Bispecific Antibodies were Assayed for TDCC Efficacy Against NUGC4_D4, SNU620, IM95 and HEK293/hCLDN 18.1 cells using CytoTox 96® non-radioactive cytotoxicity assay kit (Promega, G1780). T cells were isolated from human PBMCs using human total T cell isolation kit (Miltenyi, 130-096-535). The human T cells and the target cells were resuspended in medium (RPMI1640+5% FBS). The target cell density was adjusted to 3× 10⁵ cells/mL, and the T cell density was adjusted to 1.2×10⁶/mL. 50 μL of each type of the cells was added to the wells of a 96-well plate (effector-to-target ratio of 4:1). A test antibody was diluted with medium (RPMI1640+5% FBS) to different concentrations, and 50 μL was added to the wells of the 96-well plate. The samples were incubated at 37° C. for 48 h, and then 10× Triton-X 100 lysate (RPMI1640+5% FBS+10% Triton-X 100) was added to the target cell maximum LDH release control well and volume correction control well. The mixture was well mixed and incubated at 37° C. for 0.5 h. The 96-well plate was centrifuged at 400 g for 4 min. 50 μL of the supernatant was taken, and then LDH chromogenic solution was added at a concentration of 50 μL/well. After the mixture was left standing in the absence of light at room temperature for 20 min, the plate was read on MD StakMax (OD490). PR004549 was used as a positive control for CLDN18.2 target cells, PR004313 as a positive control for CLDN18.1 target cells, and PR004312 antibody as a negative control. For the calculation of the results, corrected readings were calculated first. The reading of the medium background control well was subtracted from the readings of the experimental wells, target cell spontaneous release LDH control well and effector cell spontaneous release LDH control well, and then the reading of the volume correction control well was subtracted from the reading of the target cell maximum LDH release control well. TDCC activity (%)=(corrected reading of experimental well-corrected reading of effector cell spontaneous release LDH control well-corrected reading of target cell spontaneous release LDH control well)/(corrected reading of target cell maximum LDH release control well-corrected reading of target cell spontaneous release LDH control well)×100. FIG. 6 shows the TDCC activity of the test antibodies, and the values are detailed in Table 19. In NUGC4_D8 cells highly endogenously expressing CLDN18.2, the test antibodies can induce higher or comparable TDCC activity compared to PR004549. Whereas, in IM95 cells lowly expressing CLDN18.2, the test antibodies induce lower TDCC activity compared to PR004549. SNU620 cells have a mutation from methionine to leucine at amino acid 149 of CLDN18.2, representing a subset of gastric cancer patients with the CLDN18.2 mutation, and the test antibodies can induce higher or comparable TDCC activity compared to PR004549. The test antibodies failed to elicit a TDCC effect against HEK293/hCLDN 18.1 cells.

TABLE 19
The TDCC activity of CLDN18.2 × CD3 bispecific antibodies

| | Titer EC₅₀ (pM) | | |
|---|---|---|---|
| Antibody | NUGC4_D8 | IM95 | SNU620 |
| PR004549 | 32.0-502 | 109-159 | 55.9-66.2 |
| PR004603 | 251 | NA | 17.8 |
| PR005072 | 142 | NA | 34.5 |
| PR005076 | 102 | | |
| PR005397 | 10.0 | NA | 31.6 |
| PR005398 | 9.00 | NA | 18.3 |
| PR005399 | 14.8 | | 41.1 |
| PR005401 | 6.40 | | 25.8 |
| PR005411 | 11.0 | NA | 38.5 |
| PR005422 | 21.9 | NA | 313.0 |
| PR005518 | 30.6 | | |
| PR005519 | 32.9 | NA | 70.5 |
| PR005520 | 22.4 | | |
| PR005521 | 73.9 | NA | 41.0 |
| PR005522 | 186 | | |
| PR005525 | 28.9 | | |
| PR005354 | 11.0 | | |
| PR006023 | 48.5 | | |
| PR006292 | 185 | NA | 112.7 |
| PR006293 | 87.0 | 460 | 18.5 |
| PR006384 | 57.9 | 938 | 57.1 |

### Example 12: Binding Affinity of Antibodies to Human Fc Receptor Proteins by BLI Method

The binding kinetics between proteins and antibodies was analyzed by the Biolayer Interferometry (BLI) technique using an Octet Red 96e (Fortebio) system. The rotation speed was set at 1000 rpm for the system. 10× kinetics buffer (ForteBio, Cat #18-1105) was diluted to 1× kinetics buffer for affinity assay and dilution of samples. FAB2G sensor (Fortebio, 18-5125) arranged in line was equilibrated in a test buffer for 10 min and then used to capture antibodies (PR002725, PR005397, PR006384, PR005411, PR006292, PR006023 and PR006293) at a capture height of 1 nm. After equilibrated in the buffer for 120 s, the FAB2G sensor was allowed to bind to 2-fold serially diluted human Fc receptor protein. The protein concentration and binding and dissociation time are shown in Table 20. Finally, the FAB2G sensor was immersed in a 10 mM glycine-hydrochloric acid solution at pH 1.5 for regeneration to elute the proteins bound to the sensor. The affinity assays of antibodies for FcRn were performed under the conditions of both pH 6.0 buffer and pH 7.4 buffer. For PR004549, the capture sensor is ProL (Fortebio, 18-5085). When data analysis was performed using Octet Data Analysis software (Fortebio, version 11.0), 0 nM was used as a reference hole, and reference subtraction was performed; the “1:1 Global fitting” method was selected to fit the data, and the kinetics parameters of the binding of proteins to antibodies were calculated, with kon(1/Ms) values, kdis(1/s) values and KD(M) values obtained. For the interactions of fast binding and fast dissociation, the “steady state” method was selected to fit the data to obtain KD(M) values. The binding affinity of the antibodies to human Fc receptor proteins is shown in Table 21.

TABLE 20
Information about the Fc receptor proteins
and experimental parameters

| | | | | Binding |
|---|---|---|---|---|
| | | | Test | time/ |
| Fc receptor | Catalog | | concentration | dissociation |
| protein | No. | Manufacturer | (nM) | time |
| Human CD64 | CM60 | NovoProtein | 200-12.5* | 60 s/20 s |
| Human FcRn | CI01 | Scientific | 400-25 | 60 s/20 s |
| | | Co., Ltd. | (pH 6.0) | |
| | | | 1000-62.5 | |
| | | | (pH 7.4) | |
| Human CD32a | CS35 | | 10000-625 | 30 s/20 s |
| Human CD32b | C444 | | 10000-625 | 30 s/20 s |
| Human | CS11 | | 10000-625 | 60 s/20 s |
| CD16a(F) | | | | |
| Human | C441 | | 6000-375 | 60 s/20 s |
| CD16a(V) | | | | |

*For antibody PR002725, the test concentration of CD64 was 40-1.25 nM

TABLE 21
The binding affinity of antibodies to Human Fc receptor proteins

| | | Concentration | | | | |
|---|---|---|---|---|---|---|
| Fc receptor | Antibody | (nM) | KD(M) | kon(1/Ms) | kdis(1/s) | Full R\hat{ }2 |
| Human CD64 | PR002725 | 40-1.25 | 9.13E−11 | 1.42E+06 | 1.30E−04 | 0.9978 |
| | PR004549 | 200-25 | 9.62E−06 | 6.00E+04 | 5.77E−01 | 0.9814 |
| | PR005397 | 200-12.5 | 5.06E−08 | 8.66E+05 | 4.38E−02 | 0.9951 |
| | PR006384 | 800-50 | NA¹ | | | |
| | PR005411 | 200-12.5 | 4.71E−08 | 8.85E+05 | 4.17E−02 | 0.9934 |
| | PR006292 | 800-50 | NA¹ | | | |
| | PR006023 | 200-12.5 | 5.39E−08 | 8.74E+05 | 4.71E−02 | 0.9952 |
| | PR006293 | 800-50 | NA¹ | | | |
| Human | PR002725 | 400-25 | 1.29E−07 | 5.85E+05 | 7.57E−02 | 0.9944 |
| CD16a(V) | PR004549 | 6000-375 | NA¹ | | | |
| | PR005397 | 6000-375 | 1.80E−06 | NA² | NA² | 0.9218 |
| | PR006384 | 6000-375 | NA¹ | | | |
| | PR005411 | 6000-375 | 2.30E−06 | NA² | NA² | 0.9126 |
| | PR006292 | 6000-375 | NA¹ | | | |
| | PR006023 | 6000-375 | 3.00E−06 | NA² | NA² | 0.9002 |
| | PR006293 | 6000-375 | NA¹ | | | |
| Human | PR002725 | 10000-625 | 2.10E−06 | NA² | NA² | 0.9884 |
| CD16a(F) | PR004549 | | NA¹ | | | |
| | PR005397 | | | | | |
| | PR006384 | | | | | |
| | PR005411 | | | | | |
| | PR006292 | | | | | |
| | PR006023 | | | | | |
| | PR006293 | | | | | |
| Human FcRn | PR002725 | 400-25 | 1.55E−07 | 3.46E+05 | 5.37E−02 | 0.9906 |
| pH 6.0 | PR004549 | 400-25 | 2.06E−07 | 2.80E+05 | 5.77E−01 | 0.9852 |
| | PR005397 | 400-25 | 1.82E−07 | 3.22E+05 | 5.86E−02 | 0.9907 |
| | PR006384 | 400-25 | 2.10E−07 | 2.89E+05 | 6.08E−02 | 0.9898 |
| | PR005411 | 400-25 | 2.15E−07 | 3.00E+05 | 6.44E−02 | 0.9879 |
| | PR006292 | 400-25 | 2.00E−07 | 3.05E+05 | 6.09E−02 | 0.9895 |
| | PR006023 | 400-25 | 2.10E−07 | 2.98E+05 | 6.26E−02 | 0.9889 |
| | PR006293 | 400-25 | 2.17E−07 | 2.91E+05 | 6.32E−02 | 0.9886 |
| Human FcRn | PR002725 | 1000-62.5 | NA¹ | | | |
| pH 7.4 | PR004549 | | | | | |
| | PR005397 | | | | | |
| | PR006384 | | | | | |
| | PR005411 | | | | | |
| | PR006292 | | | | | |
| | PR006023 | | | | | |
| | PR006293 | | | | | |
| Human | PR002725 | 10000-625 | 7.40E−07 | NA² | NA² | 0.9828 |
| CD32a(H) | PR004549 | | NA¹ | | | |
| | PR005397 | | | | | |
| | PR006384 | | | | | |
| | PR005411 | | | | | |
| | PR006292 | | | | | |
| | PR006023 | | | | | |
| | PR006293 | | | | | |
| Human CD32b | PR002725 | 10000-625 | 2.60E−06 | NA² | NA² | 0.9981 |

| | |
|---|---|
| PR004549 | NA¹ |
| PR005397 | |
| PR006384 | |
| PR005411 | |
| PR006292 | |
| PR006023 | |
| PR006293 | |

NA¹: There is no binding of the antibody to the protein
NA²: The antibody binds to the receptor protein in a fast binding and fast dissociation mode; the fitting method adopted is steady state fitting, without kon/kdis values.

### Example 13: Binding Affinity of Antibodies to CD3 Proteins of Different Species by BLI Method

The binding kinetics between proteins and antibodies was analyzed by the Biolayer Interferometry (BLI) technique using an Octet Red 96e (Fortebio) system. Human CD3E (Acro, CDE-H5223), cynomolgus CD3E (Acro, CDE-C5226) and murine CD3E (Acro, CDE-M5256) were mixed with biotin (Thermo Scientific, A39257) in a molar ratio of 1:3. The mixture was incubated at 4° C. overnight, and then excess biotin was removed to obtain biotinylated CD3E. The rotation speed was set at 1000 rpm for the system. 10× kinetics buffer (ForteBio, Cat #18-1105) was diluted to 1× kinetics buffer for affinity assay and dilution of samples. SA sensor (Fortebio, 18-5019) arranged in line was equilibrated in a test buffer for 10 min and then used to capture CD3 with a biotin label or biotinylated CD3 at a capture height of 0.2 nm. After being equilibrated in the buffer for 120 s, the SA sensor with captured CD3 was allowed to bind to 2-fold serially diluted antibodies. The antibody concentration is shown in Table 22, and binding and dissociation time were set to 180 s and 300 s. Finally, the SA sensor was immersed in a 10 mM glycine-hydrochloric acid solution at pH 1.5 for regeneration to elute the antibodies bound to the sensor. Antibody anti-CD3e 48-2B (santa cruz biotechnology,s SC-1174) is a murine positive antibody. When data analysis was performed using Octet Data Analysis software (Fortebio, version 11.0), 0 nM was used as a reference hole, and reference subtraction was performed; the “1:1 Global fitting” method was selected to fit the data, and the kinetics parameters of the binding of proteins to antibodies were calculated, with kon (1/Ms) values, kdis (1/s) values and KD(M) values obtained. For the interactions of fast binding and fast dissociation, the “steady state” method was selected to fit the data to obtain KD(M) values. The binding affinity of the antibodies to CD3 proteins of different species is shown in Table 23. PR002199 was derived from the anti-BCMA (TNB308902)×CD3 (TNB_F2B) bispecific antibody in the patent WO2018052503 of Teneobio. PR004931 was derived from the anti-CEA×CD3 bispecific antibody in the patent WO2017055389A1 of Roche.

TABLE 22
Information about the CD3 receptor proteins
and experimental parameters

| Protein | Catalog No. | Manufacturer |
|---|---|---|
| Biotinylated human CD3E&D | CDD-H82W6 | Acro |
| Human CD3Ec | CDE-H5223 | biosystems |
| Biotinylated cynomolgus CD3E&D | CDD-C82W6 | |
| Cynomolgus CD3E | CDE-C5226 | |
| Biotinylated murine CD3E&D | CDD-M82W5 | |

TABLE 23
The binding affinity of the antibodies to CD3 proteins of different species

| | | Concentration | | | | |
|---|---|---|---|---|---|---|
| Protein | Antibody | (nM) | KD(M) | kon(1/Ms) | kdis(1/s) | Full R\hat{ }2 |
| Human CD3E | PR004549 | 20-1.25 | 4.10E−09 | 1.59E+05 | 6.52E−04 | 0.9986 |
| | PR004931 | 100-6.25 | 3.08E−08 | 7.31E+04 | 2.25E−03 | 0.9992 |
| | PR006292 | 100-6.25 | 9.90E−08 | 9.89E+04 | 9.78E−03 | 0.9971 |
| | PR006293 | 100-6.25 | 3.17E−08 | 6.81E+04 | 2.16E−03 | 0.9993 |
| | PR006384 | 100-6.25 | 1.51E−07 | 8.54E+04 | 1.29E−02 | 0.9981 |
| Biotinylated | PR002199 | 200-12.5 | 2.41E−07 | 2.15E+05 | 5.18E−02 | 0.9834 |
| human CD3E&D | PR004549 | 30-1.875 | 6.15E−09 | 9.65E+04 | 5.93E−04 | 0.9988 |
| | PR004931 | 100-6.25 | 3.37E−08 | 4.07E+04 | 1.37E−03 | 0.9973 |
| | PR006292 | 100-6.25 | 1.44E−07 | 8.47E+04 | 1.22E−02 | 0.9972 |
| | PR006384 | 100-6.25 | 1.32E−07 | 9.02E+04 | 1.19E−02 | 0.9985 |
| Cynomolgus | PR004549 | 20-1.25 | 9.75E−09 | 4.03E+04 | 3.93E−04 | 0.9998 |
| CD3E | PR004931 | 100-6.25 | 2.59E−08 | 7.03E+04 | 1.82E−03 | 0.9995 |
| | PR006292 | 100-6.25 | 1.40E−07 | 7.51E+04 | 1.05E−02 | 0.9952 |
| | PR006293 | 100-6.25 | 3.24E−08 | 6.07E+04 | 1.97E−03 | 0.9995 |
| | PR006384 | 100-6.25 | 1.65E−07 | 7.40E+04 | 1.22E−02 | 0.9970 |
| Biotinylated | PR004549 | 30-1.875 | 1.30E−08 | 1.79E+04 | 2.33E−04 | 0.9995 |
| cynomolgus | PR004931 | 100-6.25 | 2.13E−08 | 4.16E+04 | 8.83E−04 | 0.9972 |
| CD3E&D | PR006292 | 100-6.25 | 1.13E−07 | 8.57E+04 | 9.65E−03 | 0.9991 |
| | PR006384 | 100-6.25 | 1.52E−07 | 6.22E+04 | 9.42E−03 | 0.9986 |
| Biotinylated | anti-CD3e(48-2B) | 40-2.5 | 1.71E−10 | 8.03E+05 | 1.37E−04 | 0.9940 |

| | | | |
|---|---|---|---|
| murine CD3E&D | PR004549 | 120-7.5 | NA¹ |
| | PR006384 | 120-7.5 | |
| | PR006292 | 120-7.5 | |
| | PR004931 | 120-7.5 | |

NA¹: There is no binding of the antibody to the protein

### Example 14: Binding Affinity of CLDN18.2×CD3 Bispecific Antibodies to Human CLDN18.2 Paralogous Proteins

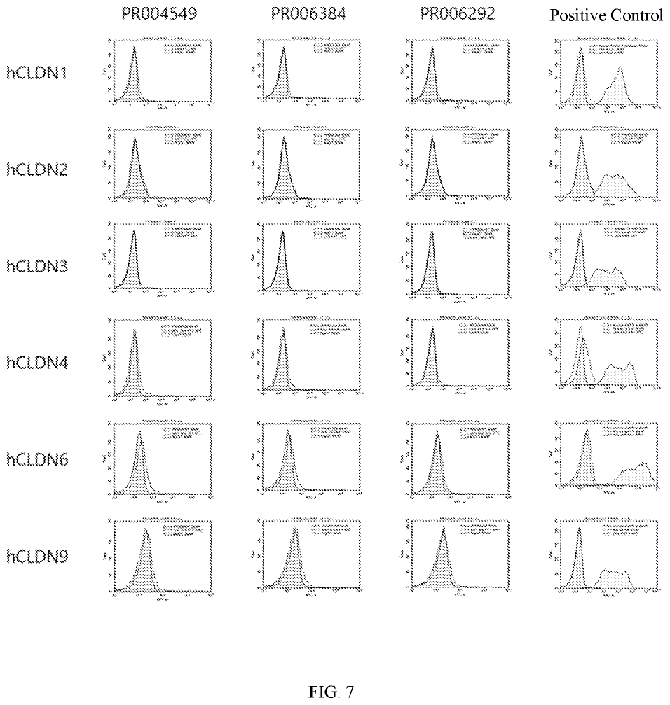

Antibodies were assayed for binding affinity to human CLDN18.2 paralogous proteins by FACS (ACEA NovoCyte). CLDN1, CLDN2, CLDN3, CLDN4, CLDN6 and CLDN9 genes of the human CLDN18.2 paralogous family were transiently transfected into HEK293 cells. Information about plasmids is shown in Table 24. The binding affinity was determined as follows: cells were centrifuged at 300 g for 5 min and then resuspended in FACS buffer (PBS containing 2% FBS). The cell density was adjusted to 10⁶ cells/mL, and 50 μL of the cell suspension was added to each well of a 96-well plate. An antibody was diluted to 60 nM with FACS buffer, and 50 μL of the antibody dilution was added to each well of the 96-well plate. After 2 h of incubation at 4° C., the plate was washed twice with FACS buffer. Then, an FACS buffer containing a secondary antibody was added. After 1 h of incubation at 4° C., the plate was washed twice with FACS buffer. The cells were resuspended in fixative solution, and run FACS. Information about the positive control antibody, negative control antibody and secondary antibody was shown in Table 25. PR005080 was clone 1A2 antibody (produced in-house, see EP3567053A1) used as the positive control for CLDN2 binding. The FACS results are shown in FIG. 7, showing that there was no non-specific binding of PR006384 and PR006292 to CLDN1, CLDN2, CLDN3, CLDN4, CLDN6 and CLDN9 of the human CLDN18.2 paralogous family protein.

TABLE 24
Information about the expression plasmids of CLDN18.2 paralogous genes

| Name | Supplier | Catalog No. |
|---|---|---|
| pcDNA3.1_hCLDN9_NM_020982 | genscript | NM_020982.4 |
| pcDNA3.1_hCLDN6_NM_021195 | genscript | NM_021195.5 |
| pcDNA3.1_hCLDN4_NM_001305 | genscript | NM_001305.4 |
| pcDNA3.1_hCLDN3_NM_001306 | genscript | NM_001306.4 |
| pcDNA3.1_hCLDN2_NM_001171092 | genscript | NM_001171092.1 |
| pcDNA3.1_hCLDN1_NM_021101 | genscript | NM_021101.5 |

TABLE 25
Information about the flow cytometry antibodies

| Name | Supplier | Catalog No. |
|---|---|---|
| Human Claudin-1 Antibody | R&D | MAB4618 |
| Human Claudin-3 Antibody | R&D | MAB4620 |
| Human Claudin-4 Antibody | R&D | MAB4219 |
| Human Claudin-6 Antibody | R&D | MAB3656 |
| Human Claudin-9 Antibody | antibodies- | ABIN1720917 |
| | online | |
| Rat IgG2A | R&D | MAB006 |
| Mouse IgG2A | R&D | MAB003 |
| Mouse IgG2B | R&D | MAB004 |
| Rat IgG2B | R&D | MAB0061 |
| Human Claudin-2 Antibody | Produced | |
| (PR005080) | in-house | |
| Iso hIgG1 | CrownBio | C0001-4 |
| Alexa Fluor ® 647 AffiniPure | Jackson | 109-605-098 |
| Goat Anti-Human IgG, Fcγ | | |
| fragment specific | | |
| Alexa Fluor ® 647 AffiniPure | Jackson | 115-605-164 |
| Goat Anti-Mouse IgG | | |
| (subclasses 1 + 2a + 2b + 3), | | |
| Fcγ Fragment Specific | | |
| Alexa Fluor ® 647 AffiniPure | Jackson | 112-605-071 |
| Goat Anti-Rat IgG, Fcγ | | |
| fragment specific | | |

### Example 15: Binding Affinity of CLDN18.2×CD3 Bispecific Antibodies to Cynomolgus Target Proteins

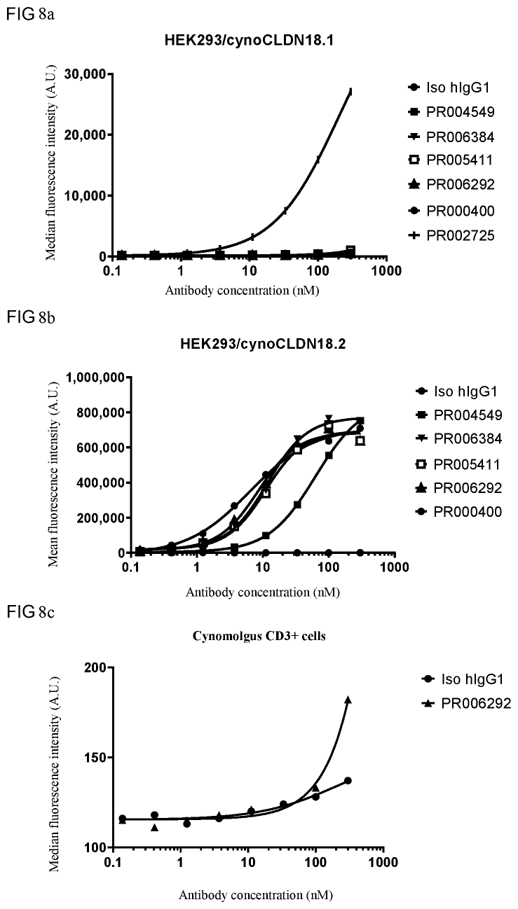

Antibodies were assayed for binding affinity by FACS. Test cells include cynomolgus CLDN18.2-expressing HEK293 cells (HEK293/cynoCLDN18.2), cynomolgus CLDN18.1-overexpressing HEK293 cells (HEK293/cynoCLDN18.1) and cynomolgus CD3 positive T cells. CD3 positive T cells were isolated from cynomolgus PBMCs using a non-human primate CD3 cell isolation kit (Miltenyi, 130-092-012). The binding affinity was determined as follows: cells were centrifuged at 300 g for 5 min and then resuspended in FACS buffer (PBS containing 2% FBS). The cell density was adjusted to 10⁶ cells/mL, and 50 μL of the cell suspension was added to each well of a 96-well plate. Antibodies were diluted to different concentrations with FACS buffer, and 50 μL of the antibody dilution was added to each well of the 96-well plate. After 2 h of incubation at 4° C., the plate was washed twice with FACS buffer. Then, an FACS buffer containing an APC-conjugated goat anti-human IgG secondary antibody (final concentration of 1.5 μg/mL, Jackson, 109-605-098) was added. After 1 h of incubation at 4° C., the plate was washed twice with FACS buffer. The cells were resuspended in fixative solution and run FACS (ACEA NovoCyte). FIG. 8 and Table 26 show the binding affinity of antibodies to HEK293 cells overexpressing cynomolgus CLDN18.1 and CLDN18.2, as well as CD3 positive T cells in cynomolgus PBMCs. All the test antibodies can bind to CLDN18.2 and CD3-expressing cells, and do not bind to CLDN18.1-expressing cells.

TABLE 26
The binding affinity of CLDN18.2 × CD3
bispecific antibodies to cells

| | Titer EC₅₀ (nM) | |
|---|---|---|
| Antibody | HEK293/cynoCLDN18.2 | CD3+ T cell |
| PR004549 | 62.8 | |
| PR005411 | 11.1 | |
| PR006292 | 8.50 | NA |
| PR006384 | 11.7 | |
| PR000400 | 6.50 | |

### Example 16: TDCC Activity of CLDN18.2×CD3 Bispecific Antibodies Against Cynomolgus CLDN18.2-Expressing Cells

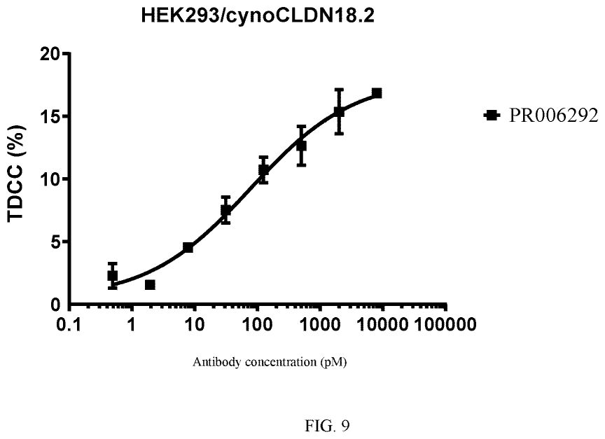

CLDN18.2×CD3 bispecific antibodies were assayed for activity of eliciting a TDCC effect against HEK293/cynoCLDN18.2 using a CytoTox 96® non-radioactive cytotoxicity assay kit (Promega, G1780). CD3 positive T cells were isolated from cynomolgus PBMCs using a non-human primate CD3 cell isolation kit (Miltenyi, 130-092-012). The cynomolgus T cells and target cells were resuspended in medium (RPMI1640+5% FBS). The target cell density was adjusted to 3×10⁵ cells/mL, and the T cell density was adjusted to 1.2×10⁶/mL. 50 μL of each type of the cells was added to the wells of a 96-well plate (effector-to-target ratio of 4:1). A test antibody was diluted with medium (RPMI1640+5% FBS) to different concentrations, and 50 μL was added to the wells of the 96-well plate. The samples were incubated at 37° C. for 24 h, and then 10× Triton-X 100 lysate (RPMI1640+5% FBS+10% Triton-X 100) was added to the target cell maximum LDH release control well and volume correction control well. The mixture was well mixed and incubated at 37° C. for 0.5 h. The 96-well plate was centrifuged at 400 g for 4 min. 50 μL of the supernatant was taken, and then LDH chromogenic solution was added at a concentration of 50 μL/well. After the mixture was left standing in the absence of light at room temperature for 20 min, the plate was read on MD StakMax (OD490). For the calculation of the results, corrected readings were calculated first. The reading of the medium background control well was subtracted from the readings of the experimental wells, target cell spontaneous release LDH control well and effector cell spontaneous release LDH control well, and then the reading of the volume correction control well was subtracted from the reading of the target cell maximum LDH release control well. TDCC activity (%)=(corrected reading of experimental well-corrected reading of effector cell spontaneous release LDH control well-corrected reading of target cell spontaneous release LDH control well)/(corrected reading of target cell maximum LDH release control well-corrected reading of target cell spontaneous release LDH control well)×100. FIG. 9 shows the TDCC activity induced by antibodies against HEK293/cynoCLDN18.2. The specific values are shown in Table 27.

TABLE 27
The TDCC activity of CLDN18.2 × CD3 bispecific antibodies

| | EC₅₀ (pM) |
|---|---|
| Antibody | HEK293/cynoCLDN18.2 |
| PR006292 | 76.8 |

### Example 17: In Vitro Cytokine Release Assay

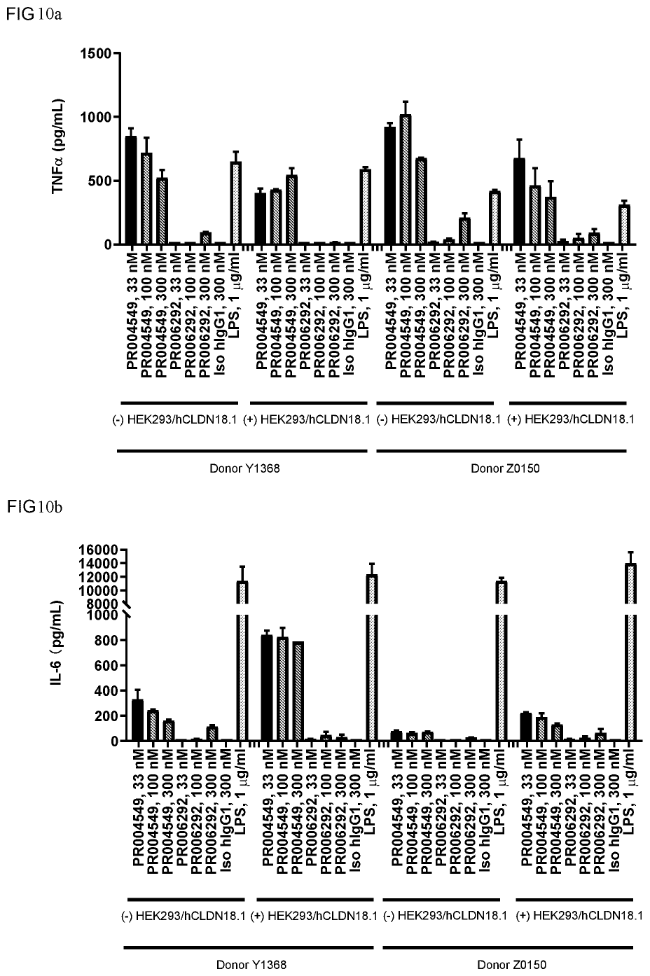

CLDN18.2×CD3 bispecific antibodies were assayed for in vitro induced cytokine release using human PBMCs, so as to predict the safety of the antibodies. PBMCs were incubated with antibodies in either presence or absence of HEK293/hCLDN18.1 cells. PBMCs and HEK293/hCLDN18.1 were resuspended in medium (RPMI1640+10% FBS). The density of HEK293/hCLDN18.1 cells was adjusted to 1.5×10⁶ cells/mL, and the cell density of PBMCs was adjusted to 2×10⁶/mL. 100 μL HEK293/hCLDN18.1 cells and 200 μL PBMCs were added to the wells of 48-well plate. Test antibodies were diluted with medium (RPMI1640+10% FBS) to different concentrations, and 100 μL was added to the wells of the 48-well plate to a final volume of 400 μL. LPS (Sigma, L6529) was used as the positive control. Iso hIgG1 (CrownBio, C0001-4) antibody was used as the negative control. The samples were incubated at 37° C. for 24 h. The supernatant was centrifuged at 300 g for 10 min, and 300 μL of the supernatant was harvested. The concentrations of IL-6 (Invitrogen, 88-7066) and TNF-α (Invitrogen, 88-7346) in the supernatant were quantified by ELISA. FIG. 10 shows cytokine release induced by antibodies in vitro. The release of IL-6 and TNF-α induced by PR004549 is higher than that induced by PR006292 in the absence of CLDN18.2 target cells, indicating better safety profile of PR006292

### Example 18: ADCC Activity of CLDN18.2×CD3 Bispecific Antibodies

CLDN18.2×CD3 bispecific antibodies were assayed for activity of eliciting ADCC effect against Jurkat cells and HEK293/hCLDN 18.2 using a CytoTox 96® non-radioactive cytotoxicity assay kit (Promega, G1780). Human PBMCs were centrifuged at 300 g for 10 min and cultured in a medium (RPMI1640+10% FBS) overnight. NK cells were isolated from the human PBMCs using a human NK cell isolation kit (Miltenyi, 130-092-657). Jurkat cells were centrifuged at 300 g for 5 min and human NK cells at 300 g for 10 min. Then the cells were resuspended in medium (RPMI1640+5% FBS). The target cell density was adjusted to 3× 10⁵ cells/mL, and the NK cell density was adjusted to 1.8×10⁶/mL. 50 μL of each type of the cells was added to the wells of 96-well plate (effector-to-target ratio of 6:1). Test antibodies were diluted with medium (RPMI1640+5% FBS) to different concentrations, and 50 μL was added to the wells of the 96-well plate. The samples were incubated at 37° C. for 5 h, and then 10× Triton-X 100 lysate (RPMI1640+5% FBS+10% Triton-X 100) was added to the target cell maximum LDH release control well and volume correction control well. The mixture was well mixed and incubated at 37° C. for 0.5 h. The 96-well plate was centrifuged at 300 g for 5 min. 50 μL of the supernatant was taken, and then LDH chromogenic solution was added at a concentration of 50 μL/well. After the mixture was left standing in the absence of light at room temperature for 20 min, the plate was read on MD StakMax (OD490). PR003767 was used as the positive control and Iso hIgG1 (CrownBio, C0001-4) antibody as the negative control. For the calculation of the results, corrected readings were calculated first. The reading of the medium background control well was subtracted from the readings of the experimental wells, target cell spontaneous release LDH control well and effector cell spontaneous release LDH control well, and then the reading of the volume correction control well was subtracted from the reading of the target cell maximum LDH release control well. ADCC activity (%)=(corrected reading of experimental well-corrected reading of effector cell spontaneous release LDH control well-corrected reading of target cell spontaneous release LDH control well)/(corrected reading of target cell maximum LDH release control well-corrected reading of target cell spontaneous release LDH control well)×100. FIG. 11(a) shows the ADCC activity of antibodies against Jurkat cells. PR006292 and PR004549 were unable to elicit ADCC effects against Jurkat cells.

CLDN18.2×CD3 bispecific antibodies were assayed for activity of eliciting an ADCC effect against NUGC4_D8 using NK92/CD16a cells. NUGC4_D8 and NK92/CD16a were resuspended in a medium (RPMI1640+5% FBS). The target cell density was adjusted to 3×10⁵ cells/mL, and the NK92/CD16a cell density was adjusted to 1.8×10⁶/mL. 50 μL of each type of the cells was added to the wells of a 96-well plate (effector-to-target ratio of 6:1). Test antibodies were diluted with medium (RPMI1640+5% FBS) to different concentrations, and 50 μL was added to the wells of the 96-well plate. The samples were incubated at 37° C. for 5 h. PR003197 was used as a positive control. FIG. 11(b) shows the ADCC activity of antibodies against NUGC4_D8. PR006292 and PR004549 were unable to elicit ADCC effects against NUGC4_D8 cells.

### Example 19: CDC Activity of CLDN18.2×CD3 Bispecific Antibodies

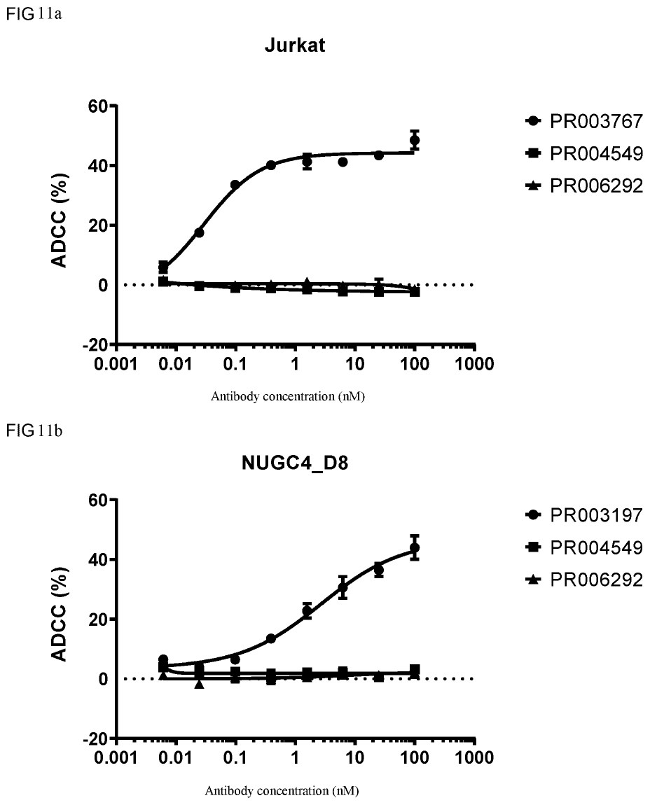

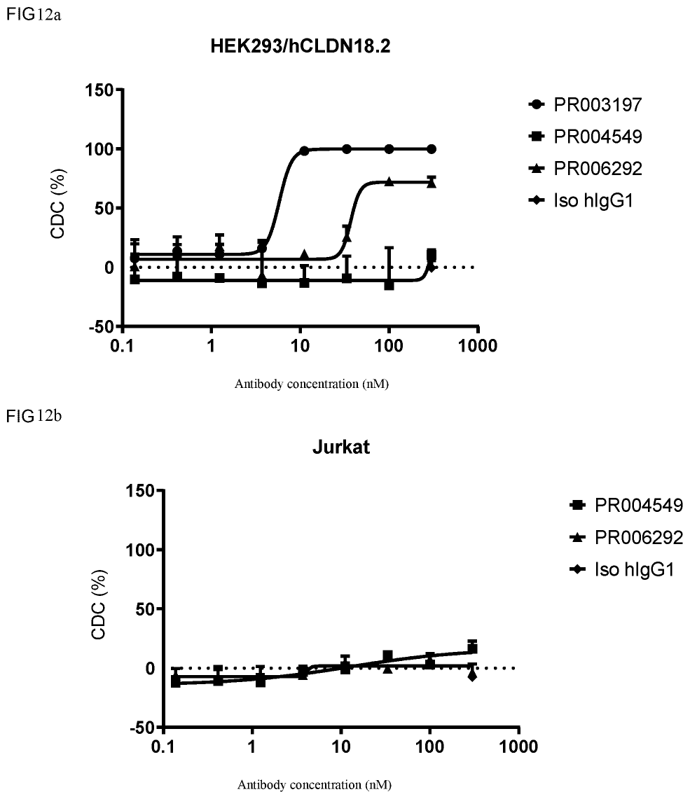

CLDN18.2 antibodies were assayed for CDC effects against HEK293/hCLDN18.2 and Jurkat cells using a CellTiter-Glo luminescent cell viability assay kit (Promega, G7573). The target cells were centrifuged at 300 g for 5 min and then resuspended in RPMI1640 serum-free medium. The target cell density was adjusted to 2×10⁵ cells/mL, and 25 μL of the cell suspension was added to each well of a 96-well plate. The antibodies were diluted to different concentrations with the serum-free medium, and 25 μL of the antibody dilution was added to each well of the 96-well plate. 50 μL of normal human serum (Access cell culture, 515) was added to a final concentration of 50%, and the resulting mixture was incubated at 37° C. for 24 h. The 96-well plate was left standing at room temperature for 30 min, and 100 μL of CellTiter-Glo chromogenic solution was added to each well at room temperature. Then, the sample was incubated in the absence of light at room temperature for 10 min. The plate was read with PE Enspire. CDC activity (%)=[1−(luminescent sample)/(luminescent mock control)]×100. An IMAB362 analogue was used as a positive control and Iso hIgG1 (CrownBio, C0001-4) antibody as the negative control. FIG. 12 shows the CDC activity of PR006292 antibody against Jurkat cells and human CLDN18.2-overexpressing HEK293 cells. PR006292 induced a greater CDC effect than that induced by PR004549 in HEK293/hCLDN18.2, whereas no CDC activity was observed in Jurkat cells. The CDC activity of CLDN18.2×CD3 bispecific antibodies is specifically shown in Table 28.

TABLE 28
The CDC activity of CLDN18.2 × CD3 bispecific antibodies

| Cell | | PR006292 | PR004549 |
|---|---|---|---|
| HEK293/hCLDN18.2 | Maximum | 71.8 | 0.00 |
| | killing | | |
| | rate (%) | | |
| | EC₅₀ (nM) | 37.1 | NA |
| Jurkat | Maximum | 0.00 | 0.00 |
| | killing | | |
| | rate (%) | | |
| | EC₅₀ (nM) | NA | NA |

### Example 20: Competitive Binding Activity of CLDN18.2×CD3 Bispecific Antibodies

This example is to study the binding of anti-human CLDN18.2 bispecific antibodies to the epitope region of a human CLDN18.2 antigen. Competitive binding experiments were performed at the cellular level using human CLDN18.2-overexpressing HEK293/hCLDN18.2 cells. Briefly, the anti-human CLDN18.2 antibodies PR000400 and PR004549 were biotinylated using a biotinylation kit (ThermoFisher, A35358) according to the instructions. To a 96-well V-bottom plate (Corning, 3894) were added cells at 2×10⁶ cells/mL and a suspension of human CLDN18.2-overexpressing HEK293T/hCLDN18.2 cells at 50 μL/well, followed by the addition of 25 μL of biotinylated anti-human CLDN18.2 antibody PR000400 or PR004549. The mixture was well mixed and incubated at 4° C. for 30 min. 25 μL of the corresponding serially diluted non-biotinylated anti-human CLDN18.2 antibody was added. The mixture was well mixed and incubated at 4° C. for 1 h. The cells in each well were washed twice with 200 μL of pre-cooled FACS buffer (2% BSA in DPBS) and centrifuged at 500 g at 4° C. for 5 min, and then the supernatant was discarded. A fluorescent secondary antibody (Jackson ImmunoResearch, 016-540-084, 1:500) was added. The mixture was incubated at 4° C. in the absence of light for 1 h. The cells in each well were washed twice with 200 μL of pre-cooled FACS buffer (2% BSA in DPBS) and centrifuged at 500 g at 4° C. for 5 min, and then the supernatant was discarded. Finally, the cells in each well were resuspended in 200 μL of pre-cooled FACS buffer, and the fluorescence signal values were read using a ACEA_NovoCyte. The inhibition rate was calculated using the formula, inhibition rate (%)=(A−B)/A×100 (note: A: fluorescence signal after interaction of biotinylated antibody with ISO (hIgG1) (Crownbio, c0001-4); B: fluorescence signal after interaction of biotinylated antibody with non-biotinylated antibody).

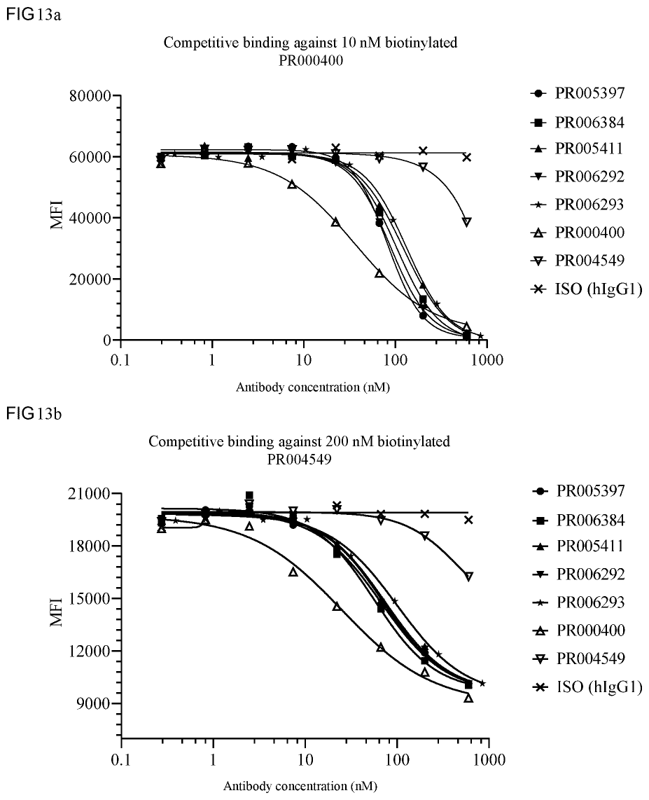

As shown in FIG. 13 and Table 29, the anti-CLDN18.2 bispecific antibodies of the present invention are all able to block the binding of PR000400 or PR004549 to human CLDN18.2, and the detected blocking ability of the antibodies increases with the antibody concentration in a positively correlated manner, indicating that these antibodies have very similar epitopes to those of PR000400 and PR004549. The test antibodies have low binding affinity for HEK293/hCLDN18.1 cells. From the above results, it can be inferred that the test antibodies bind to human CLDN18.2 protein at ECL1 (Extracellular loop 1) rather than ECL2.

TABLE 29
The competitive binding ability of CLDN18.2 antibodies
against antibodies PR000400 and PR004549

| | 10 nM biotinylated | | 200 nM biotinylated | |
|---|---|---|---|---|
| | PR000400 | | PR004549 | |
| | | Maximum | | Maximum |
| | | inhibition | | inhibition |
| | IC50 | rate | IC50 | rate |
| Antibody | (nM) | (%) | (nM) | (%) |
| PR005397 | 81.8 | 98.4 | 67.5 | 48.0 |
| PR006384 | 103 | 97.8 | 56.7 | 48.5 |
| PR005411 | 122 | 96.2 | 74.4 | 48.0 |
| PR006292 | 87.7 | 97.7 | 70.8 | 48.1 |
| PR006293 | 132 | 97.8 | 100 | 48.0 |
| PR000400 | 36.2 | 92.7 | 25.2 | 52.3 |
| PR004549 | NA | 35.5 | NA | 16.7 |
| ISO hIgG1 | NA | 0.00 | NA | 0.00 |

### Example 21: Pharmacokinetics Study on CLDN18.2×CD3 Bispecific Antibodies

Pharmacokinetics studies were carried out as follows using BALB/c nude mice. 6 female BALB/c nude mice weighing 18-22 g were selected and received an antibody dosing by intravenous injection at dose of 5 mg/kg. The whole blood of 3 mice in one group was collected prior to the administration and 15 min, 24 h (1 day), 4 days and 10 days after the administration, and the whole blood of 3 mice in the other group was collected prior to the administration and 5 h, 2 days, 7 days and 14 days after the administration. The whole blood was left standing for 30 min to coagulate, and then centrifuged. The isolated serum sample was cryopreserved at −80° C. until it was taken for analysis. The drug concentration in the mouse serum was quantified by ELISA. The total ELISA (total method) was performed by capturing human Fc-containing antibodies in mouse serum using goat anti-human Fc polyclonal antibody and detected by HRP-labeled goat anti-human Fc secondary antibody. The CLDN18.2 binding domain ELISA (Free X method) was performed by capturing CLDN18.2 binding domain-containing antibodies in the mouse serum using CLDN18.2 protein and detected by HRP-labeled goat anti-human Fc secondary antibody. The plasma concentration data were analyzed using Phoenix WinNonlin software (version 8.2) by non-compartmental analysis (NCA) to evaluate the pharmacokinetic parameters.

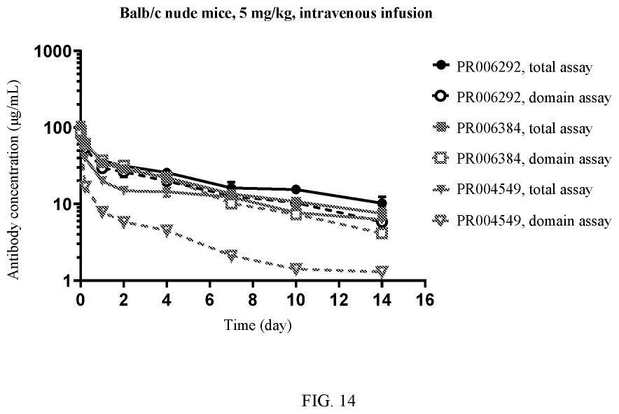

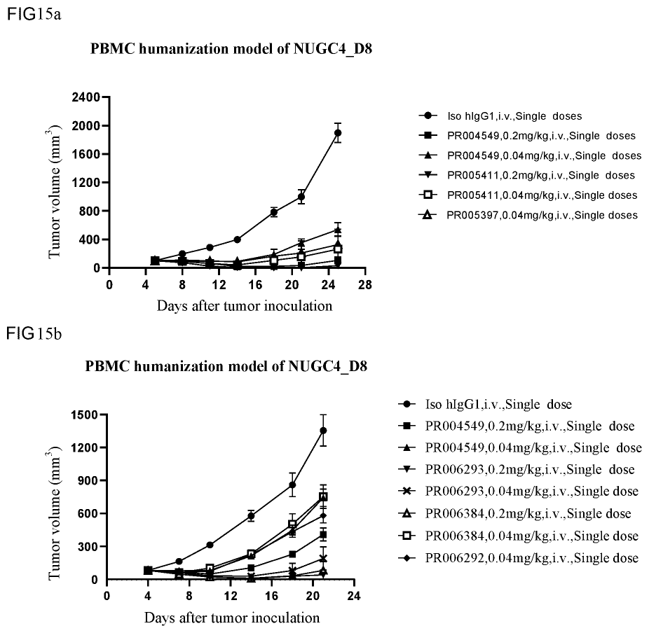

The pharmacokinetics of PR006292, PR006384 and PR004549 is shown in FIG. 14, and the pharmacokinetic parameters are shown in Table 30. PR006292 and PR006384 are superior to PR004549 in stability in mice, and have long half-lives and high drug exposure amount in mice.

TABLE 30
Pharmacokinetic parameters of PR006292, PR006384 and PR004549

| | PR004549 | | PR006292 | | PR006384 | |
|---|---|---|---|---|---|---|
| Method | Total assay | Domain assay | Total assay | Domain assay | Total assay | Domain assay |
| T_{1/2} (h) | 7,395 ± 606 | 5,714 ± 464 | 7,395 ± 606 | 5,714 ± 464 | 6,352 ± 325 | 5,751 ± 192 |
| V_{d} (mL/kg) | 100 | 77.2 | 100 | 77.2 | 100 | 90.5 |
| AUCₐₗₗ (μg/mL h) | 106 | 94.1 | 106 | 94.1 | 106 | 89.7 |
| AUC (%)* | 121 | 132 | 121 | 132 | 141 | 112 |
| Cl (mL/h/kg) | 0.5 | 0.73 | 0.5 | 0.73 | 0.59 | 0.76 |
| C₀ (μg/mL) | 173 | 135 | 173 | 135 | 198 | 130 |

*AUC (%) = AUC/AUC_{(total assay; mean value)} × 100

### Example 22: In Vivo Pharmacodynamics Studies on CLDN18.2×CD3 Bispecific Antibodies

### NUGC4_D8 Tumor Model

In vivo pharmacodynamics studies were carried out by using NCG mice to re-establish the NUGC4_D8 tumor model of human PBMC immune system. The method is specifically as follows. On the day of cell inoculation, each NCG mouse was subcutaneously inoculated with NUGC4_D8 cells and PBMCs. When the mean tumor volume of each group of mice reached 90 mm³, the mice were divided into groups, and a total of one administration was performed through tail veins. After the start of administration, the body weight and the tumor volume were measured twice a week. The tumor volume was calculated as follows: tumor volume (mm³)=0.5× long diameter of tumor×short diameter of tumor2. The data were analyzed using t-test.

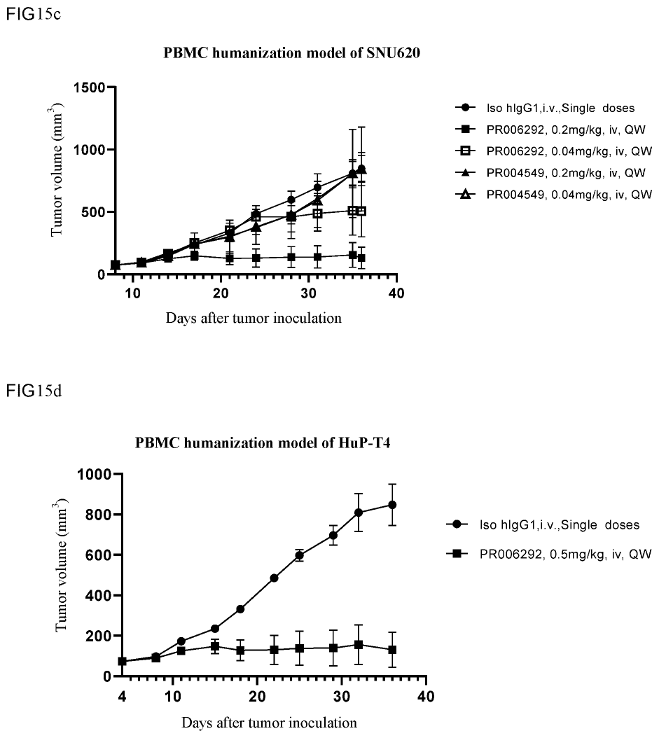

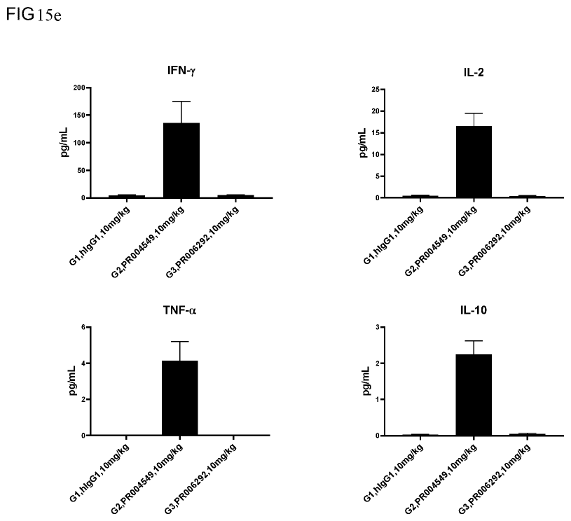

The in vivo anti-tumor effects of PR005397, PR005411 and PR004549 are shown in FIG. 15(a). Specifically, the mean tumor volume of the Iso hIgG1 control group of mice at day 25 after inoculation was 1897 mm³. The mean tumor volume of the test drug PR004549 (0.2 mg/kg) treatment group at day 25 after inoculation was 104 mm³, showing a significant difference (p value <0.0001) from that of the Iso hIgG1 control group, with the tumor growth inhibition rate TGI (%) being 94.48%. The mean tumor volume of the test drug PR004549 (0.04 mg/kg) treatment group at day 25 after inoculation was 538 mm³, showing a significant difference (p value <0.0001) from that of the Iso hIgG1 control group, with the tumor growth inhibition rate TGI (%) being 71.61%. The mean tumor volume of the test drug PR005411 (0.2 mg/kg) treatment group at day 25 after inoculation was 30 mm³, showing a significant difference (p value <0.0001) from that of the Iso hIgG1 control group, with the tumor growth inhibition rate TGI (%) being 98.39%. The mean tumor volume of the test drug PR005411 (0.04 mg/kg) treatment group at day 25 after inoculation was 263 mm³, showing a significant difference (p value <0.0001) from that of the Iso hIgG1 control group, with the tumor growth inhibition rate TGI (%) being 86.1%. The mean tumor volume of the test drug PR005397 (0.04 mg/kg) treatment group at day 25 after inoculation was 327 mm³, showing a significant difference (p value <0.0001) from that of the Iso hIgG1 control group, with the tumor growth inhibition rate TGI (%) being 82.75%. Throughout the treatment, the animals showed good tolerance for the drugs, with no great weight loss and animals' death occurred. The in vivo anti-tumor effects of PR005397 and PR005411 are superior to that of PR004549.

The in vivo anti-tumor effects of PR006292, PR006293, PR006384 and PR004549 are shown in FIG. 15(b). Specifically, the mean tumor volume of the Iso hIgG1 control group of mice at day 25 after inoculation was 1355 mm³. The mean tumor volume of the test drug PR004549 (0.2 mg/kg) treatment group at day 25 after inoculation was 408 mm³, showing a significant difference (p value=0.0001) from that of the Iso hIgG1 control group, with the tumor growth inhibition rate TGI (%) being 69.83%. The mean tumor volume of the test drug PR004549 (0.04 mg/kg) treatment group at day 25 after inoculation was 743 mm³, showing a significant difference (p value=0.0037) from that of the Iso hIgG1 control group, with the tumor growth inhibition rate TGI (%) being 45.15%. The mean tumor volume of the test drug PR006293 (0.2 mg/kg) treatment group at day 25 after inoculation was 39 mm³, showing a significant difference (p value <0.0001) from that of the Iso hIgG1 control group, with the tumor growth inhibition rate TGI (%) being 97.06%. The mean tumor volume of the test drug PR006293 (0.04 mg/kg) treatment group at day 25 after inoculation was 190 mm³, showing a significant difference (p value <0.0001) from that of the Iso hIgG1 control group, with the tumor growth inhibition rate TGI (%) being 85.96%. The mean tumor volume of the test drug PR006384 (0.2 mg/kg) treatment group at day 25 after inoculation was 81 mm³, showing a significant difference (p value <0.0001) from that of the Iso hIgG1 control group, with the tumor growth inhibition rate TGI (%) being 94%. The mean tumor volume of the test drug PR006384 (0.04 mg/kg) treatment group at day 25 after inoculation was 752 mm³, showing a significant difference (p value=0.0071) from that of the Iso hIgG1 control group, with the tumor growth inhibition rate TGI (%) being 44.47%. The mean tumor volume of the test drug PR006292 (0.04 mg/kg) treatment group at day 25 after inoculation was 580 mm³, showing a significant difference (p value=0.0006) from that of the Iso hIgG1 control group, with the tumor growth inhibition rate TGI (%) being 57.15%. Throughout the treatment, the animals showed good tolerance for the drugs, with no great weight loss and animals' death occurred. The in vivo anti-tumor effects of PR006292, PR006293 and PR006384 are superior to that of PR004549.

### SNU620 PBMC Tumor Model

In vivo pharmacodynamics studies were carried out by using NCG mice to re-establish the SNU620 tumor model of human PBMC immune system. The method is specifically as follows. On the day of cell inoculation, each NCG mouse was subcutaneously inoculated with SNU620 tumor cells. When the mean tumor volume of each group of mice reached 70 mm³, the mice were divided into groups, and a total of four administrations were performed through tail veins. After the start of administration, the body weight and the tumor volume were measured twice a week. The tumor volume was calculated as follows: tumor volume (mm³)=0.5× long diameter of tumor×short diameter of tumor2. The data were analyzed using t-test.

The in vivo anti-tumor effects of PR006292 and PR004549 are shown in FIG. 15(c). Specifically, the mean tumor volume of the Iso hIgG1 control group of mice at day 36 after inoculation was 847 mm³. The mean tumor volume of the test drug PR006292 (0.2 mg/kg) treatment group at day 36 after inoculation was 131 mm³, showing a significant difference (p value=0.0076) from that of the Iso hIgG1 control group, with the tumor growth inhibition rate TGI (%) being 84.53%. The mean tumor volume of the test drug PR006292 (0.04 mg/kg) treatment group at day 36 after inoculation was 505 mm³, showing no significant difference (p value=0.3856) from that of the Iso hIgG1 control group, with the tumor growth inhibition rate TGI (%) being 40.36%. The mean tumor volume of the test drug PR004549 (0.2 mg/kg) treatment group at day 36 after inoculation was 858 mm³, showing a significant difference (p value >0.9999) from that of the Iso hIgG1 control group, with the tumor growth inhibition rate TGI (%) being-1.28%. The mean tumor volume of the test drug PR004549 (0.04 mg/kg) treatment group at day 36 after inoculation was 844 mm³, showing a significant difference (p value >0.9999) from that of the Iso hIgG1 control group, with the tumor growth inhibition rate TGI (%) being 0.39%. Throughout the treatment, the animals showed good tolerance for the drugs, with no great weight loss and animals' death occurred.

### HuP-T4 PBMC Tumor Model

In vivo pharmacodynamics studies were carried out by using NCG mice to re-establish the HuP-T4 tumor model of human PBMC immune system. The method is specifically as follows. On the day of cell inoculation, each NCG mouse was subcutaneously inoculated with HuP-T4 tumor cells. When the mean tumor volume of each group of mice reached 130 mm³, the mice were divided into groups, and a total of four administrations were performed through tail veins. After the start of administration, the body weight and the tumor volume were measured twice a week. The tumor volume was calculated as follows: tumor volume (mm³)=0.5× long diameter of tumor×short diameter of tumor2. The data were analyzed using t-test.

The in vivo anti-tumor effect of PR006292 is shown in FIG. 15(d). Specifically, the mean tumor volume of the Iso hIgG1 control group of mice at day 36 after inoculation was 1059 mm³. The mean tumor volume of the test drug PR006292 (0.5 mg/kg) treatment group at day 36 after inoculation was 129 mm³, showing a significant difference (p value=0.0022) from that of the Iso hIgG1 control group, with the tumor growth inhibition rate TGI (%) being 87.75%. Throughout the treatment, the animals showed good tolerance for the drugs, with no great weight loss and animals' death occurred.

### In Vivo Cytokine Release Test in Mouse

In vivo cytokine storm studies were carried out by using NCG mice to re-establish the human PBMC immune system. The method is specifically as follows. Each NCG mouse was intravenously injected with 2×10⁷ human PBMCs, and at next day, intravenously administered with PR006292, PR004549 and control IgG1 antibodies. Blood was taken to collect serum at hr 0 (before administration), 4 and 24 hrs after administration. The MSD method was utilized to detect the levels of a plurality of cytokines in the serum, including IFN-γ, IL-10, IL-12p70, IL-13, IL-1B, IL-2, IL-4, IL-6, IL-8 and TNF-α. The data were analyzed using t-test.

FIG. 15(e) shows the expression of a part of the cytokines that were detectable in the mouse serum 4 hrs after injection of the antibodies. The results showed that PR006292 induced a lower release of the cytokines such as IFN-γ, IL-2 and TNF-α compared to the control antibody PR004549, indicating a better safety.

1. A CLDN18.2-targeting antibody comprising a heavy chain variable region comprising HCDR1, HCDR2 and HCDR3, wherein the HCDR1 comprises an amino acid sequence as set forth in any one of SEQ ID NOs: 16-18, the HCDR2 comprises an amino acid sequence as set forth in any one of SEQ ID NOs: 42-46 and SEQ ID NOs: 48-54, and the HCDR3 comprises an amino acid sequence as set forth in any one of SEQ ID NOs: 77-82.

2. The antibody according to claim 1, wherein the HCDR1, the HCDR2 and the HCDR3 comprise amino acid sequences as set forth in SEQ ID NO: 16, SEQ ID NO: 42 and SEQ ID NO: 77, respectively; or
the HCDR1, the HCDR2 and the HCDR3 comprise amino acid sequences as set forth in SEQ ID NO: 16, SEQ ID NO: 43 and SEQ ID NO: 78, respectively; or
the HCDR1, the HCDR2 and the HCDR3 comprise amino acid sequences as set forth in SEQ ID NO: 16, SEQ ID NO: 44 and SEQ ID NO: 79, respectively; or
the HCDR1, the HCDR2 and the HCDR3 comprise amino acid sequences as set forth in SEQ ID NO: 17, SEQ ID NO: 45 and SEQ ID NO: 80, respectively; or
the HCDR1, the HCDR2 and the HCDR3 comprise amino acid sequences as set forth in SEQ ID NO: 18, SEQ ID NO: 43 and SEQ ID NO: 80, respectively; or
the HCDR1, the HCDR2 and the HCDR3 comprise amino acid sequences as set forth in SEQ ID NO: 18, SEQ ID NO: 43 and SEQ ID NO: 78, respectively; or
the HCDR1, the HCDR2 and the HCDR3 comprise amino acid sequences as set forth in SEQ ID NO: 16, SEQ ID NO: 43 and SEQ ID NO: 81, respectively; or
the HCDR1, the HCDR2 and the HCDR3 comprise amino acid sequences as set forth in SEQ ID NO: 16, SEQ ID NO: 46 and SEQ ID NO: 82, respectively; or
the HCDR1, the HCDR2 and the HCDR3 comprise amino acid sequences as set forth in SEQ ID NO: 16, SEQ ID NO: 48 and SEQ ID NO: 78, respectively; or
the HCDR1, the HCDR2 and the HCDR3 comprise amino acid sequences as set forth in SEQ ID NO: 16, SEQ ID NO: 49 and SEQ ID NO: 78, respectively; or
the HCDR1, the HCDR2 and the HCDR3 comprise amino acid sequences as set forth in SEQ ID NO: 16, SEQ ID NO: 50 and SEQ ID NO: 78, respectively; or
the HCDR1, the HCDR2 and the HCDR3 comprise amino acid sequences as set forth in SEQ ID NO: 16, SEQ ID NO: 51 and SEQ ID NO: 78, respectively; or
the HCDR1, the HCDR2 and the HCDR3 comprise amino acid sequences as set forth in SEQ ID NO: 16, SEQ ID NO: 52 and SEQ ID NO: 78, respectively; or
the HCDR1, the HCDR2 and the HCDR3 comprise amino acid sequences as set forth in SEQ ID NO: 16, SEQ ID NO: 53 and SEQ ID NO: 79, respectively; or
the HCDR1, the HCDR2 and the HCDR3 comprise amino acid sequences as set forth in SEQ ID NO: 16, SEQ ID NO: 54 and SEQ ID NO: 78, respectively.

3. The antibody according to claim 1, wherein the heavy chain variable region further comprises framework regions, among which the HFR1 comprises an amino acid sequence as set forth in SEQ ID NO: 6 or 7, the HFR2 comprises an amino acid sequence as set forth in any one of SEQ ID NOs: 28-34, the HFR3 comprises an amino acid sequence as set forth in any one of SEQ ID NOs: 63-68, and the HFR4 comprises an amino acid sequence as set forth in any one of SEQ ID NOs: 84 and 86-89.

4. The antibody according to claim 3, wherein the heavy chain variable region comprises an amino acid sequence as set forth in any one of SEQ ID NOs: 150-157 and SEQ ID NOs: 159-165.

5. The antibody according to claim 1, wherein the antibody further comprises a heavy chain constant region; preferably, the heavy chain constant region is selected from hIgG1, hIgG2, hIgG3 and hIgG4 and a variant thereof; more preferably, the heavy chain constant region is hIgG1.

6. The antibody according to claim 1, wherein the antibody is a full-length antibody, an Fab, an Fab′, an F(ab′)₂, an Fv, an scFv, a bispecific antibody, a multispecific antibody, a heavy-chain antibody or a single-domain antibody, or a monoclonal or polyclonal antibody prepared from the antibodies above.

7. The antibody according to claim 6, wherein the antibody is a single-domain antibody comprising an amino acid sequence as set forth in any one of SEQ ID NOs: 182-189 and SEQ ID NOs: 191-197.

8. A bispecific antibody comprising a first protein functional region targeting CD3 and a second protein functional region targeting CLDN18.2; wherein
the first protein functional region is in the form of an Fab, and the second protein functional region is in the form of VHs and preferably comprises 2 or 3 VHs; when the second protein functional region comprises 3 VHs linked in series, the first protein functional region and the second protein functional region are each linked to an Fc's double strand; when the second protein functional region comprises 2 VHs linked in series, the first protein functional region and the second protein functional region are each linked to an Fc's double strand; when the second protein functional region comprises 3 VHs and one of the 3 VHs is linked to the first protein functional region, the remaining two VHs are linked in series, and the first protein functional region and the two VHs linked in series of the second protein functional region are each linked to an Fc's double strand;
alternatively, the first protein functional region is in the form of Fab and the second protein functional region is in the form of HCAb;
alternatively, the first protein functional region is in the form of Fab and the second protein functional region is in the form of VH-HCAb, the second protein functional region preferably comprising a total of 4 VHs.

9. The bispecific antibody according to claim 8, wherein the second protein functional region comprises a heavy chain variable region comprising HCDR1, HCDR2 and HCDR3, wherein the HCDR1 comprises an amino acid sequence as set forth in any one of SEQ ID NOS: 16-18, the HCDR2 comprises an amino acid sequence as set forth in any one of SEQ ID NOs: 42-46 and SEQ ID NOs: 48-54, and the HCDR3 comprises an amino acid sequence as set forth in any one of SEQ ID NOs: 77-82;
preferably, the HCDR1, the HCDR2 and the HCDR3 comprise amino acid sequences as set forth in SEQ ID NO: 16, SEQ ID NO: 42 and SEQ ID NO: 77, respectively; or the HCDR1, the HCDR2 and the HCDR3 comprise amino acid sequences as set forth in SEQ ID NO: 16, SEQ ID NO: 43 and SEQ ID NO: 78, respectively; or the HCDR1, the HCDR2 and the HCDR3 comprise amino acid sequences as set forth in SEQ ID NO: 16, SEQ ID NO: 44 and SEQ ID NO: 79, respectively; or the HCDR1, the HCDR2 and the HCDR3 comprise amino acid sequences as set forth in SEQ ID NO: 16, SEQ ID NO: 48 and SEQ ID NO: 78, respectively; or
the HCDR1, the HCDR2 and the HCDR3 comprise amino acid sequences as set forth in SEQ ID NO: 16, SEQ ID NO: 49 and SEQ ID NO: 78, respectively; or the HCDR1, the HCDR2 and the HCDR3 comprise amino acid sequences as set forth in SEQ ID NO: 16, SEQ ID NO: 50 and SEQ ID NO: 78, respectively; or the HCDR1, the HCDR2 and the HCDR3 comprise amino acid sequences as set forth in SEQ ID NO: 16, SEQ ID NO: 51 and SEQ ID NO: 78, respectively; or
the HCDR1, the HCDR2 and the HCDR3 comprise amino acid sequences as set forth in SEQ ID NO: 16, SEQ ID NO: 52 and SEQ ID NO: 78, respectively; or the HCDR1, the HCDR2 and the HCDR3 comprise amino acid sequences as set forth in SEQ ID NO: 16, SEQ ID NO: 53 and SEQ ID NO: 79, respectively; or the HCDR1, the HCDR2 and the HCDR3 comprise amino acid sequences as set forth in SEQ ID NO: 16, SEQ ID NO: 54 and SEQ ID NO: 78, respectively;
more preferably, the heavy chain variable region comprises an amino acid sequence as set forth in any one of SEQ ID NOs: 150-152 and SEQ ID NOs: 159-165.

10. The bispecific antibody according to claim 8, wherein the first protein functional region comprises a light chain variable region comprising LCDR1, LCDR2 and LCDR3 as set forth in SEQ ID NO: 101, SEQ ID NO: 116 and SEQ ID NO: 131, respectively, and a heavy chain variable region comprising HCDR1, HCDR2 and HCDR3 as set forth in SEQ ID NO: 11, SEQ ID NO: 38 and SEQ ID NO: 72, respectively;
preferably, the heavy chain variable region comprises an amino acid sequence as set forth in SEQ ID NO: 149 or SEQ ID NO: 144 and the light chain variable region comprises an amino acid sequence as set forth in SEQ ID NO: 168.

11. The bispecific antibody according to claim 8, wherein
the bispecific antibody comprises three polypeptide chains in the following forms:
two N-termini of the Fc are linked to the Fab and the VH, respectively; preferably, the bispecific antibody has a first polypeptide chain as shown in formula: VH_{CLDN18.2}-VH_{CLDN18.2}-hinge-CH2-CH3 or VH_{CLDN18.2}-VH_{CLDN18.2}-VH_{CLDN18.2}-hinge-CH2-CH3, a second polypeptide chain as shown in formula: VH_{CD3}-CH1-hinge-CH2-CH3 and a third polypeptide chain as shown in formula: VL_{CD3}-CL;
alternatively, one C-terminus of the HCAb is linked to a VH or VL of the Fab; preferably, the bispecific antibody has a first polypeptide chain as shown in formula: VH_{CLDN18.2}-hinge-CH2-CH3, a second polypeptide chain as shown in formula: VH_{CLDN18.2}-hinge-CH2-CH3-VH_{CD3}-CH1 and a third polypeptide chain as shown in formula: VL_{CD3}-CL; alternatively, the bispecific antibody has a first polypeptide chain as shown in formula: VH_{CLDN18.2}-hinge-CH2-CH3, a second polypeptide chain as shown in formula: VH_{CLDN18.2}-hinge-CH2-CH3-VL_{CD3}-CL and a third polypeptide chain as shown in formula: VH_{CD3}-CH1; alternatively, the bispecific antibody has a first polypeptide chain as shown in formula: VH_{CLDN18.2}-VH_{CLDN18.2}-hinge-CH2-CH3, a second polypeptide chain as shown in formula: VH_{CLDN18.2}-VH_{CLDN18.2}-hinge-CH2-CH3-VH_{CD3}-CH1 and a third polypeptide chain as shown in formula: VL_{CD3}-CL; alternatively, the bispecific antibody has a first polypeptide chain as shown in formula: VH_{CLDN18.2}-VH_{CLDN18.2}-hinge-CH2-CH3, a second polypeptide chain as shown in formula: VH_{CLDN18.2}-VH_{CLDN18.2}-hinge-CH2-CH3-VL_{CD3}-CL and a third polypeptide chain as shown in formula: VH_{CD3}-CH1;
alternatively, the N-terminus of the heavy chain of the Fab is linked to one VH_{CLDN18.2}, the C-terminus of the heavy chain is linked to one N-terminus of the Fc, and the C-terminus of the VHs linked in series is linked to the other N-terminus of the Fc; preferably, the bispecific antibody has a first polypeptide chain as shown in formula: VH_{CLDN18.2}-VH_{CLDN18.2}-hinge-CH2-CH3, a second polypeptide chain as shown in formula: VH_{CLDN18.2}-VH_{CD3}-CH1-hinge-CH2-CH3 and a third polypeptide chain as shown in formula: VL_{CD3}-CL;
more preferably, different functional units such as VH, CH2-CH3 and VL are operably linked by linker peptides preferably comprising an amino acid sequence as set forth in any one of SEQ ID NOs: 244-248, preferably the sequence set forth in SEQ ID NO: 246;
more preferably, two N-termini of the Fc are linked to the Fab and the VH, respectively; preferably, the bispecific antibody has a first polypeptide chain as shown in formula: VH_{CLDN18.2}-linker peptide-VH_{CLDN18.2}-hinge-CH2-CH3 or VH_{CLDN18.2}-linker peptide-VH_{CLDN18.2}-linker peptide-VH_{CLDN18.2}-hinge-CH2-CH3, a second polypeptide chain as shown in formula: VH_{CD3}-CH1-hinge-CH2-CH3 and a third polypeptide chain as shown in formula: VL_{CD3}-CL;
alternatively, one C-terminus of the HCAb is linked to a VH or VL of the Fab; preferably, the bispecific antibody has a first polypeptide chain as shown in formula: VH_{CLDN18.2}-hinge-CH2-CH3, a second polypeptide chain as shown in formula: VH_{CLDN18.2}-hinge-CH2-CH3-linker peptide-VH_{CD3}-CH1 and a third polypeptide chain as shown in formula: VL_{CD3}-CL; alternatively, the bispecific antibody has a first polypeptide chain as shown in formula: VH_{CLDN18.2}-hinge-CH2-CH3, a second polypeptide chain as shown in formula: VH_{CLDN18.2}-hinge-CH2-CH3-linker peptide-VL_{CD3}-CL and a third polypeptide chain as shown in formula: VH_{CD3}-CH1;
alternatively, one C-terminus of the VH-HCAb is linked to a VH or VL of the Fab; preferably, the bispecific antibody has a first polypeptide chain as shown in formula: VH_{CLDN18.2}-linker peptide-VH_{CLDN18.2}-hinge-CH2-CH3, a second polypeptide chain as shown in formula: VH_{CLDN18.2}-linker peptide-VH_{CLDN18.2}-hinge-CH2-CH3-linker peptide-VH_{CD3}-CH1 and a third polypeptide chain as shown in formula: VL_{CD3}-CL; alternatively, the bispecific antibody has a first polypeptide chain as shown in formula: VH_{CLDN18.2}-linker peptide-VH_{CLDN18.2}-hinge-CH2-CH3, a second polypeptide chain as shown in formula: VH_{CLDN18.2}-linker peptide-VH_{CLDN18.2}-hinge-CH2-CH3-linker peptide-VL_{CD3}-CL and a third polypeptide chain as shown in formula: VH_{CD3}-CH1;
alternatively, the N-terminus of the heavy chain of the Fab is linked to one VH_{CLDN18.2}, the C-terminus of the heavy chain is linked to one N-terminus of the Fc, and the C-terminus of the VHs linked in series is linked to the other N-terminus of the Fc; preferably, the bispecific antibody has a first polypeptide chain as shown in formula: VH_{CLDN18.2}-linker peptide-VH_{CLDN18.2}-hinge-CH2-CH3, a second polypeptide chain as shown in formula: VH_{CLDN18.2}-linker peptide-VH_{CD3}-CH1-hinge-CH2-CH3 and a third polypeptide chain as shown in formula: VL_{CD3}-CL.

12. The bispecific antibody according to claim 11, wherein the first polypeptide chain comprises an amino acid sequence as set forth in SEQ ID NO: 214, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 219, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 220, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 221, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 222, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 223, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 224, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 225, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 226, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 227, the second polypeptide chain comprises an amino acid sequence as set forth in SEQ ID NO: 228, and the third polypeptide chain comprises an amino acid sequence as set forth in SEQ ID NO: 200;
alternatively, the first polypeptide chain comprises an amino acid sequence as set forth in SEQ ID NO: 227, the second polypeptide chain comprises an amino acid sequence as set forth in SEQ ID NO: 230, and the third polypeptide chain comprises an amino acid sequence as set forth in SEQ ID NO: 229;
alternatively, the first polypeptide chain comprises an amino acid sequence as set forth in SEQ ID NO: 219, the second polypeptide chain comprises an amino acid sequence as set forth in SEQ ID NO: 231, and the third polypeptide chain comprises an amino acid sequence as set forth in SEQ ID NO: 200;
alternatively, the first polypeptide chain comprises an amino acid sequence as set forth in SEQ ID NO: 219, the second polypeptide chain comprises an amino acid sequence as set forth in SEQ ID NO: 232, and the third polypeptide chain comprises an amino acid sequence as set forth in SEQ ID NO: 229;
alternatively, the first polypeptide chain comprises an amino acid sequence as set forth in SEQ ID NO: 219, the second polypeptide chain comprises an amino acid sequence as set forth in SEQ ID NO: 233, and the third polypeptide chain comprises an amino acid sequence as set forth in SEQ ID NO: 200;
alternatively, the first polypeptide chain comprises an amino acid sequence as set forth in SEQ ID NO: 234, the second polypeptide chain comprises an amino acid sequence as set forth in SEQ ID NO: 213, and the third polypeptide chain comprises an amino acid sequence as set forth in SEQ ID NO: 200;
alternatively, the first polypeptide chain comprises an amino acid sequence as set forth in SEQ ID NO: 219, the second polypeptide chain comprises an amino acid sequence as set forth in SEQ ID NO: 209, and the third polypeptide chain comprises an amino acid sequence as set forth in SEQ ID NO: 200;
alternatively, the first polypeptide chain comprises an amino acid sequence as set forth in SEQ ID NO: 221, the second polypeptide chain comprises an amino acid sequence as set forth in SEQ ID NO: 209, and the third polypeptide chain comprises an amino acid sequence as set forth in SEQ ID NO: 200;
alternatively, the first polypeptide chain comprises an amino acid sequence as set forth in SEQ ID NO: 236, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 236, the second polypeptide chain comprises an amino acid sequence as set forth in SEQ ID NO: 237, and the third polypeptide chain comprises an amino acid sequence as set forth in SEQ ID NO: 200;
alternatively, the first polypeptide chain comprises an amino acid sequence as set forth in SEQ ID NO: 238, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 239, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 240, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 241, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 242, the second polypeptide chain comprises an amino acid sequence as set forth in SEQ ID NO: 235, and the third polypeptide chain comprises an amino acid sequence as set forth in SEQ ID NO: 200;
alternatively, the first polypeptide chain comprises an amino acid sequence as set forth in SEQ ID NO: 243, the second polypeptide chain comprises an amino acid sequence as set forth in SEQ ID NO: 235, and the third polypeptide chain comprises an amino acid sequence as set forth in SEQ ID NO: 200.

13. An isolated nucleic acid encoding the antibody according to claim 1.

14. A recombinant expression vector comprising the isolated nucleic acid according to claim 13, wherein preferably, the recombinant expression vector is a plasmid, a cosmid, a phage or a viral vector, wherein the viral vector is preferably a retroviral vector, a lentiviral vector, an adenoviral vector or an adeno-associated viral vector.

15. A transformant comprising the recombinant expression vector according to claim 14 in a host cell, wherein preferably, the host cell is an E. coli TG1, BL21 cell, or CHO-K1 cell.

16. A chimeric antigen receptor comprising the antibody according to claim 1.

17. A genetically modified cell comprising the antibody according to claim 1, wherein preferably, the genetically modified cell is a eukaryotic cell, preferably an isolated human cell, and more preferably an immune cell such as a T cell or an NK cell.

18. A method for preparing a bispecific antibody comprising culturing the transformant according to claim 15, and obtaining the antibody or the bispecific antibody from the culture.

19. An antibody-drug conjugate comprising a cytotoxic agent and the antibody according to claim 1, wherein preferably, the cytotoxic agent is MMAF or MMAE.

20. A pharmaceutical composition comprising the antibody according to claim 1 or the bispecific antibody according to claim 8, and a pharmaceutically acceptable carrier; wherein
preferably, the pharmaceutical composition further comprises one or more of the group consisting of a hormonal agent, a small molecule-targeted agent, a proteasome inhibitor, an imaging agent, a diagnostic agent, a chemotherapeutic agent, an oncolytic drug, a cytotoxic agent, a cytokine, an activator of a costimulatory molecule, an inhibitor of an inhibitory molecule, and a vaccine.

21. Use of the antibody according to claim 1 in the manufacture of a medicament for the prevention or treatment of a CD3 and/or CLDN18.2-associated disease, wherein
the disease is preferably cancer, wherein the cancer is preferably breast cancer, ovarian cancer, endometrial cancer, renal cancer, melanoma, lung cancer, gastric cancer, liver cancer, esophageal cancer, cervical cancer, head and neck tumor, cholangiocarcinoma, gallbladder cancer, bladder cancer, sarcoma or colorectal cancer; preferably, the cancer is breast cancer, ovarian cancer, endometrial cancer, renal cancer or cholangiocarcinoma; more preferably, the cancer is breast cancer.

22. A kit comprising the antibody according to claim 1;
preferably, the kit further comprises (i) a device for administering the antibody or an antigen-binding fragment thereof or the antibody-drug conjugate or the pharmaceutical composition; and/or (ii) instructions.

23. A kit of parts comprising a kit A and a kit B, wherein:
the kit A comprises the antibody according to claim 1;
the kit B comprises other anti-tumor antibodies or a pharmaceutical composition comprising the other anti-tumor antibodies, and/or one or more of the group consisting of a hormonal agent, a small molecule-targeted agent, a proteasome inhibitor, an imaging agent, a diagnostic agent, a chemotherapeutic agent, an oncolytic drug, a cytotoxic agent, a cytokine, an activator of a co-stimulatory molecule, an inhibitor of an inhibitory molecule, and a vaccine.

24. A method for diagnosing, treating and/or preventing a CLDN18.2-mediated disease or disorder comprising administering to a patient in need thereof a therapeutically effective amount of the antibody according to claim 1, or treating a patient in need thereof with the kit of parts according to claim 23.

25. The method according to claim 24, wherein the disease or disorder is a tumor, preferably a CLDN18.2 positive tumor, and more preferably gastric cancer, esophageal cancer, lung cancer, ovarian cancer, melanoma, renal cancer, breast cancer, colorectal cancer, liver cancer, pancreatic cancer, bladder cancer, head and neck cancer, bronchial carcinoma, glioma and/or leukemia.

26. A method for immunodetection or determination of CLDN18.2 comprising using the antibody according to claim 1, wherein preferably, the detection is for non-diagnostic and/or therapeutic purposes.

27. A combination therapy comprising administering to a patient in need thereof the antibody according to claim 1, and a second therapeutic agent, wherein the second therapeutic agent preferably comprises other anti-tumor antibodies or a pharmaceutical composition comprising the other anti-tumor antibodies, and/or one or more of the group consisting of a hormonal agent, a small molecule-targeted agent, a proteasome inhibitor, an imaging agent, a diagnostic agent, a chemotherapeutic agent, an oncolytic drug, a cytotoxic agent, a cytokine, an activator of a co-stimulatory molecule, an inhibitor of an inhibitory molecule, and a vaccine.
